SEQUENCE LISTING (44 sequences)

SEQ ID NO: 1 - protein, 529 aa, Homo sapiens [sequence omitted]

SEQ ID NO: 2 - protein, 118 aa, Homo sapiens
MPREDAHFIYGYPKKGHGHSYTTAEEAAGIGILTVILGVLLLIGCWYCRRRNGYRALMDKSLHVGTQCALTRRCPQEGFDHRDSKVSLQEKNCEPVVPNAPPAYEKLSAEQSPPPYSP

SEQ ID NO: 3 - protein, 223 aa, Homo sapiens
MNGDDAFARRPTVGAQIPEKIQKAFDDIAKYFSKEEWEKMKASEKIFYVYMKRKYEAMTKLGFKATLPPFMCNKRAEDFQGNDLDNDPNRGNQVERPQMTFGRLQGISPKIMPKKPAEEGNDSEEVPEASGPQNDGKELCPPGKPTTSEKIHERSGNREAQEKEERRGTAHRWSSQNTHNIGRFSLSTSMGAVHGTPKTITHNRDPKGGNMPGPTDCVRENSW

SEQ ID NO: 4 - protein, 750 aa, Homo sapiens
MWNLLHETDSAVATARRPRWLCAGALVLAGGFFLLGFLFGWFIKSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 5 - protein, 309 aa, Homo sapiens
MSLEQRSLHCKPEEALEAQQEALGLVCVQAATSSSSPLVLGTLEEVPTAGSTDPPQSPQGASAFPTTINFTRQRQPSEGSSSREEEGPSTSCILESLFRAVITKKVADLVGFLLLKYRAREPVTKAEMLESVIKNYKHCFPEIFGKASESLQLVFGIDVKEADPTGHSYVLVTCLGLSYDGLLGDNQIMPKTGFLIIVLVMIAMEGGHAPEEEIWEELSVMEVYDGREHSAYGEPRKLLTQDLVQEKYLEYRQVPDSDPARYEFLWGPRALAETSYVKVLEYVIKVSARVRFFFPSLREAALREEEEGV

SEQ ID NO: 6 - protein, 314 aa, Homo sapiens
MPLEQRSQHCKPEEGLEARGEALGLVGAQAPATEEQEAASSSSTLVEVTLGEVPAAESPDPPQSPQGASSLPTTMNYPLWSQSYEDSSNQEEEGPSTFPDLESEFQAALSRKVAELVHFLLLKYRAREPVTKAEMLGSVVGNWQYFFPVIFSKASSSLQLVFGIELMEVDPIGHLYIFATCLGLSYDGLLGDNQIMPKAGLLIIVLAIIAREGDCAPEEKIWEELSVLEVFEGREDSILGDPKKLLTQHFVQENYLEYRQVPGSDPACYEFLWGPRALVETSYVKVLHHMVKISGGPHISYPPLHEWVLREGEE

SEQ ID NO: 7 - protein, 180 aa, Homo sapiens
MQAEGRGTGGSTGDADGPGGPGIPDGPGGNAGGPGEAGATGGRGPRGAGAARASGPGGGAPRGPHGGAASGLNGCCRCGARGPESRLLEFYLAMPFATPMEAELARRSLAQDAPPLPVPGVLLKEFTVSGNILTIRLTAADHRQLQLSISSCLQQLSLLMWITQCFLPVFLAQPPSGQRR

SEQ ID NO: 8 - protein, 509 aa, Homo sapiens
MERRRLWGSIQSRYISMSVWTSPRRLVELAGQSLLKDEALAIAALELLPRELFPPLFMAAFDGRHSQTLKAMVQAWPFTCLPLGVLMKGQHLHLETFKAVLDGLDVLLAQEVRPRRWKLQVLDLRKNSHQDFWTVWSGNRASLYSFPEPEAAQPMTKKRKVDGLSTEAEQPFIPVEVLVDLFLKEGACDELFSYLIEKVKRKKNVLRLCCKKLKIFAMPMQDIKMILKMVQLDSIEDLEVTCTWKLPTLAKFSPYLGQMINLRRLLLSHIHASSYISPEKEEQYIAQFTSQFLSLQCLQALYVDSLFFLRGRLDQLLRHVMNPLETLSITNCRLSEGDVMHLSQSPSVSQLSVLSLSGVMLTDVSPEPLQALLERASATLQDLVFDECGITDDQLLALLPSLSHCSQLTTLSFYGNSISISALQSLLQHLIGLSNLTHVLYPVPLESYEDIHGTLHLERLAYLHARLRELLCELGRPSMVWLSANPCPHCGDRTFYDPEPILCPCFMPN

SEQ ID NO: 9 - protein, 1255 aa, Homo sapiens [sequence omitted]

SEQ ID NO: 10 - protein, 261 aa, Homo sapiens
MWVPVVFLTLSVTWIGAAPLILSRIVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSGDSGGPLVCNGVLQGITSWGSEPCALPERPSLYTKVVHYRKWIKDTIVANP

SEQ ID NO: 11 - protein, 115 aa, Homo sapiens
MSGEPGQTSVAPPPEEVEPGSGVRIVVEYCEPCGFEATYLELASAVKEQYPGIEIESRLGGTGAFEIEINGQLVFSKLENGGFPYEKDLIEAIRRASNGETLEKITNSRPPCVIL

SEQ ID NO: 12 - protein, 153 aa, Homo sapiens
MNGDDAFARRPRDDAQISEKLRKAFDDIAKYFSKKEWEKMKSSEKIVYVYMKLNYEVMTKLGFKVTLPPFMRSKRAADFHGNDFGNDRNHRNQVERPQMTFGSLQRIFPKDPKGGNMPGPTDCVRESSWWFMKRSATLRKMTSNSPRGYDTCP

SEQ ID NO: 13 - protein, 661 aa, Homo sapiens
MDLVLKRCLLHLAVIGALLAVGATKVPRNQDWLGVSRQLRTKAWNRQLYPEWTEAQRLDCWRGGQVSLKVSNDGPTLIGANASFSIALNFPGSQKVLPDGQVIWVNNTIINGSQVWGGQPVYPQETDDACIFPDGGPCPSGSWSQKRSFVYVWKTWGQYWQVLGGPVSGLSIGTGRAMLGTHTMEVTVYHRRGSRSYVPLAHSSSAFTITDQVPFSVSVSQLRALDGGNKHFLRNQPLTFALQLHDPSGYLAEADLSYTWDFGDSSGTLISRALVVTHTYLEPGPVTAQVVLQAAIPLTSCGSSPVPGTTDGHRPTAEAPNTTAGQVPTTEVVGTTPGQAPTAEPSGTTSVQVPTTEVISTAPVQMPTAESTGMTPEKVPVSEVMGTTLAEMSTPEATGMTPAEVSIVVLSGTTAAQVTTTEWVETTARELPIPEPEGPDASSIMSTESITGSLGPLLDGTATLRLVKRQVPLDCVLYRYGSFSVTLDIVQGIESAEILQAVPSGEGDAFELTVSCQGGLPKEACMEISSPGCQPPAQRLCQPVLPSPACQLVLHQILKGGSGTYCLNVSLADTNSLAVVSTQLIMPGQEAGLGQVPLIVGILLVLMAVVLASLIYRRRLMKQDFSVPQLPHSSSHWLRLPRIFCSCPIGENSPLLSGQQV

SEQ ID NO: 14 - protein, 371 aa, Homo sapiens
MSMSPKHTTPFSVSDILSPLEESYKKVGMEGGGLGAPLAAYRQGQAAPPTAAMQQHAVGHHGAVTAAYHMTAAGVPQLSHSAVGGYCNGNLGNMSELPPYQDTMRNSASGPGWYGANPDPRFPAISRFMGPASGMNMSGMGGLGSLGDVSKNMAPLPSAPRRKRRVLFSQAQVYELERRFKQQKYLSAPEREHLASMIHLTPTQVKIWFQNHRYKMKRQAKDKAAQQQLQQDSGGGGGGGGTGCPQQQQAQQQSPRRVAVPVLVKDGKPCQAGAPAPGAASLQGHAQQQAQHQAQAAQAAAAAISVGSGGAGLGAHPGHQPGSAGQSPDLAHHAASPAALQGQVSSLSHLNSSGSDYGTMSCSTLLYGRTW

SEQ ID NO: 15 - protein, 93 aa, Homo sapiens
MKLLMVLMLAALSQHCYAGSGCPLLENVISKTINPQVSKTEYKELLQEFIDDNATTNAIDELKECFLNQTDETLSNVEVFMQLIYDSSLCDLF

SEQ ID NO: 16 - protein, 146 aa, Homo sapiens
MRLLQLLFRASPATLLLVLCLQLGANKAQDNTRKIIIKNFDIPKSVRPNDEVTAVLAVQTELKECMVVKTYLISSIPLQGAFNYKYTACLCDDNPKTFYWDFYTNRTVQIAAVVDVIRELGICPDDAAVIPIKNNRFYTIEILKVE

SEQ ID NO: 17 - protein, 622 aa, Homo sapiens
MALPTARPLLGSCGTPALGSLLFLLFSLGWVQPSRTLAGETGQEAAPLDGVLANPPNISSLSPRQLLGFPCAEVSGLSTERVRELAVALAQKNVKLSTEQLRCLAHRLSEPPEDLDALPLDLLLFLNPDAFSGPQACTRFFSRITKANVDLLPRGAPERQRLLPAALACWGVRGSLLSEADVRALGGLACDLPGRFVAESAEVLLPRLVSCPGPLDQDQQEAARAALQGGGPPYGPPSTWSVSTMDALRGLLPVLGQPIIRSIPQGIVAAWRQRSSRDPSWRQPERTILRPRFRREVEKTACPSGKKAREIDESLIFYKKWELEACVDAALLATQMDRVNAIPFTYEQLDVLKHKLDELYPQGYPESVIQHLGYLFLKMSPEDIRKWNVTSLETLKALLEVNKGHEMSPQVATLIDRFVKGRGQLDKDTLDTLTAFYPGYLCSLSPEELSSVPPSSIWAVRPQDLDTCDPRQLDVLYPKARLAFQNMNGSEYFVKIQSFLGGAPTEDLKALSQQNVSMDLATFMKLRTDAVLPLTVAEVQKLLGPHVEGLKAEERHRPVRDWILRQRQDDLDTLGLGLQGGIPNGYLVLDLSMQEALSGTPCLLGPGPVLTVLALLLASTLA

SEQ ID NO: 18 - protein, 630 aa, Homo sapiens
MALPTARPLLGSCGTPALGSLLFLLFSLGWVQPSRTLAGETGQEAAPLDGVLANPPNISSLSPRQLLGFPCAEVSGLSTERVRELAVALAQKNVKLSTEQLRCLAHRLSEPPEDLDALPLDLLLFLNPDAFSGPQACTRFFSRITKANVDLLPRGAPERQRLLPAALACWGVRGSLLSEADVRALGGLACDLPGRFVAESAEVLLPRLVSCPGPLDQDQQEAARAALQGGGPPYGPPSTWSVSTMDALRGLLPVLGQPIIRSIPQGIVAAWRQRSSRDPSWRQPERTILRPRFRREVEKTACPSGKKAREIDESLIFYKKWELEACVDAALLATQMDRVNAIPFTYEQLDVLKHKLDELYPQGYPESVIQHLGYLFLKMSPEDIRKWNVTSLETLKALLEVNKGHEMSPQAPRRPLPQVATLIDRFVKGRGQLDKDTLDTLTAFYPGYLCSLSPEELSSVPPSSIWAVRPQDLDTCDPRQLDVLYPKARLAFQNMNGSEYFVKIQSFLGGAPTEDLKALSQQNVSMDLATFMKLRTDAVLPLTVAEVQKLLGPHVEGLKAEERHRPVRDWILRQRQDDLDTLGLGLQGGIPNGYLVLDLSMQEALSGTPCLLGPGPVLTVLALLLASTLA

SEQ ID NO: 19 - protein, 123 aa, Homo sapiens
MKAVLLALLMAGLALQPGTALLCYSCKAQVSNEDCLQVENCTQLGEQCWTARIRAVGLLTVISKGCSLNCVDDSQDYYVGKKNITCCDTDLCNASGAHALQPAAAILALLPALGLLLWGPGQL

SEQ ID NO: 20 - protein, 603 aa, Homo sapiens
MSAAVTAGKLARAPADPGKAGVPGVAAPGAPAAAPPAKEIPEVLVDPRSRRRYVRGRFLGKGGFAKCFEISDADTKEVFAGKIVPKSLLLKPHQREKMSMEISIHRSLAHQHVVGFHGFFEDNDFVFVVLELCRRRSLLELHKRRKALTEPEARYYLRQIVLGCQYLHRNRVIHRDLKLGNLFLNEDLEVKIGDFGLATKVEYDGERKKTLCGTPNYIAPEVLSKKGHSFEVDVWSIGCIMYTLLVGKPPFETSCLKETYLRIKKNEYSIPKHINPVAASLIQKMLQTDPTARPTINELLNDEFFTSGYIPARLPITCLTIPPRFSIAPSSLDPSNRKPLTVLNKGLENPLPERPREKEEPVVRETGEVVDCHLSDMLQQLHSVNASKPSERGLVRQEEAEDPACIPIFWVSKWVDYSDKYGLGYQLCDNSVGVLFNDSTRLILYNDGDSLQYIERDGTESYLTVSSHPNSLMKKITLLKYFRNYMSEHLLKAGANITPREGDELARLPYLRTWFRTRSAIILHLSNGSVQINFFQDHTKLILCPLMAAVTYIDEKRDFRTYRLSLLEEYGCCKELASRLRYARTMVDKLLSSRSASNRLKAS

SEQ ID NO: 21 - protein, 232 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

SEQ ID NO: 22 - protein, 976 aa, Homo sapiens [sequence omitted]

SEQ ID NO: 23 - DNA, 2384 bp, Homo sapiens
tattgagttcttcaaacattgtagcctctttatggtctctgagaaataactaccttaaacccataatctttaatacttcctaaactttcttaataagagaagctctattcctgacactacctctcatttgcaaggtcaaatcatcattagttttgtagtctattaactgggtttgcttaggtcaggcattattattactaaccttattgttaatattctaaccataagaattaaactattaatggtgaatagagtttttcactttaacataggcctatcccactggtgggatacgagccaattcgaaagaaaagtcagtcatgtgcttttcagaggatgaaagcttaagataaagactaaaagtgtttgatgctggaggtgggagtggtattatataggtctcagccaagacatgtgataatcactgtagtagtagctggaaagagaaatctgtgactccaattagccagttcctgcagaccttgtgaggactagaggaagaatgctcctggctgttttgtactgcctgctgtggagtttccagacctccgctggccatttccctagagcctgtgtctcctctaagaacctgatggagaaggaatgctgtccaccgtggagcggggacaggagtccctgtggccagctttcaggcagaggttcctgtcagaatatccttctgtccaatgcaccacttgggcctcaatttcccttcacaggggtggatgaccgggagtcgtggccttccgtcttttataataggacctgccagtgctctggcaacttcatgggattcaactgtggaaactgcaagtttggcttttggggaccaaactgcacagagagacgactcttggtgagaagaaacatcttcgatttgagtgccccagagaaggacaaattttttgcctacctcactttagcaaagcataccatcagctcagactatgtcatccccatagggacctatggccaaatgaaaaatggatcaacacccatgtttaacgacatcaatatttatgacctctttgtctggatgcattattatgtgtcaatggatgcactgcttgggggatctgaaatctggagagacattgattttgcccatgaagcaccagcttttctgccttggcatagactcttcttgttgcggtgggaacaagaaatccagaagctgacaggagatgaaaacttcactattccatattgggactggcgggatgcagaaaagtgtgacatttgcacagatgagtacatgggaggtcagcaccccacaaatcctaacttactcagcccagcatcattcttctcctcttggcagattgtctgtagccgattggaggagtacaacagccatcagtctttatgcaatggaacgcccgagggacctttacggcgtaatcctggaaaccatgacaaatccagaaccccaaggctcccctcttcagctgatgtagaattttgcctgagtttgacccaatatgaatctggttccatggataaagctgccaatttcagctttagaaatacactggaaggatttgctagtccacttactgggatagcggatgcctctcaaagcagcatgcacaatgccttgcacatctatatgaatggaacaatgtcccaggtacagggatctgccaacgatcctatcttccttcttcaccatgcatttgttgacagtatttttgagcagtggctccgaaggcaccgtcctcttcaagaagtttatccagaagccaatgcacccattggacataaccgggaatcctacatggttccttttataccactgtacagaaatggtgatttctttatttcatccaaagatctgggctatgactatagctatctacaagattcagacccagactcttttcaagactacattaagtcctatttggaacaagcgagtcggatctggtcatggctccttggggcggcgatggtaggggccgtcctcactgccctgctggcagggcttgtgagcttgctgtgtcgtcacaagagaaagcagcttcctgaagaaaagcagccactcctcatggagaaagaggattaccacagcttgtatcagagccatttataaaaggcttaggcaatagagtagggccaaaaagcctgacctcactctaactcaaagtaatgtccaggttcccagagaatatctgctggtatttttctgtaaagaccatttgcaaaattgtaacctaatacaaagtgtagccttcttccaactcaggtagaacacacctgtctttgtcttgctgttttcactcagcccttttaacattttcccctaagcccatatgtctaaggaaaggatgctatttggtaatgaggaactgttatttgtatgtgaattaaagtgctcttatttt

SEQ ID NO: 24 - DNA, 1524 bp, Homo sapiens
agcagacagaggactctcattaaggaaggtgtcctgtgccctgaccctacaagatgccaagagaagatgctcacttcatctatggttaccccaagaaggggcacggccactcttacaccacggctgaagaggccgctgggatcggcatcctgacagtgatcctgggagtcttactgctcatcggctgttggtattgtagaagacgaaatggatacagagccttgatggataaaagtcttcatgttggcactcaatgtgccttaacaagaagatgcccacaagaagggtttgatcatcgggacagcaaagtgtctcttcaagagaaaaactgtgaacctgtggttcccaatgctccacctgcttatgagaaactctctgcagaacagtcaccaccaccttattcaccttaagagccagcgagacacctgagacatgctgaaattatttctctcacacttttgcttgaatttaatacagacatctaatgttctcctttggaatggtgtaggaaaaatgcaagccatctctaataataagtcagtgttaaaattttagtaggtccgctagcagtactaatcatgtgaggaaatgatgagaaatattaaattgggaaaactccatcaataaatgttgcaatgcatgatactatctgtgccagaggtaatgttagtaaatccatggtgttattttctgagagacagaattcaagtgggtattctggggccatccaatttctctttacttgaaatttggctaataacaaactagtcaggttttcgaaccttgaccgacatgaactgtacacagaattgttccagtactatggagtgctcacaaaggatacttttacaggttaagacaaagggttgactggcctatttatctgatcaagaacatgtcagcaatgtctctttgtgctctaaaattctattatactacaataatatattgtaaagatcctatagctctttttttttgagatggagtttcgcttttgttgcccaggctggagtgcaatggcgcgatcttggctcaccataacctccgcctcccaggttcaagcaattctcctgccttagcctcctgagtagctgggattacaggcgtgcgccactatgcctgactaattttgtagttttagtagagacggggtttctccatgttggtcaggctggtctcaaactcctgacctcaggtgatctgcccgcctcagcctcccaaagtgctggaattacaggcgtgagccaccacgcctggctggatcctatatcttaggtaagacatataacgcagtctaattacatttcacttcaaggctcaatgctattctaactaatgacaagtattttctactaaaccagaaattggtagaaggatttaaataagtaaaagctactatgtactgccttagtgctgatgcctgtgtactgccttaaatgtacctatggcaatttagctctcttgggttcccaaatccctctcacaagaatgtgcagaagaaatcataaaggatcagagattctg

SEQ ID NO: 25 - DNA, 1466 bp, Homo sapiens
gcatgctctgactttctctctctttcgattcttccatactcagagtacgcacggtctgattttctctttggattcttccaaaatcagagtcagactgctcccggtgccatgaacggagacgacgcctttgcaaggagacccacggttggtgctcaaataccagagaagatccaaaaggccttcgatgatattgccaaatacttctctaaggaagagtgggaaaagatgaaagcctcggagaaaatcttctatgtgtatatgaagagaaagtatgaggctatgactaaactaggtttcaaggccaccctcccacctttcatgtgtaataaacgggccgaagacttccaggggaatgatttggataatgaccctaaccgtgggaatcaggttgaacgtcctcagatgactttcggcaggctccagggaatctccccgaagatcatgcccaagaagccagcagaggaaggaaatgattcggaggaagtgccagaagcatctggcccacaaaatgatgggaaagagctgtgccccccgggaaaaccaactacctctgagaagattcacgagagatctggaaatagggaggcccaagaaaaggaagagagacgcggaacagctcatcggtggagcagtcagaacacacacaacattggtcgattcagtttgtcaacttctatgggtgcagttcatggtacccccaaaacaattacacacaacagggacccaaaaggggggaacatgcctggacccacagactgcgtgagagaaaacagctggtgatttatgaagagatcagcgaccctgaggaagatgacgagtaactcccctcagggatacgacacatgcccatgatgagaagcagaacgtggtgacctttcacgaacatgggcatggctgcggacccctcgtcatcaggtgcatagcaagtgaaagcaagtgttcacaacagtgaaaagttgagcgtcatttttcttagtgtgccaagagttcgatgttagcgtttacgttgtattttcttacactgtgtcattctgttagatactaacattttcattgatgagcaagacatacttaatgcatattttggtttgtgtatccatgcacctaccttagaaaacaagtattgtcggttacctctgcatggaacagcattaccctcctctctccccagatgtgactactgagggcagttctgagtgtttaatttcagattttttcctctgcatttacacacacacgcacacaaaccacaccacacacacacacacacacacacacacacacacacacacacaccaagtaccagtataagcatctgccatctgcttttcccattgccatgcgtcctggtcaagctcccctcactctgtttcctggtcagcatgtactcccctcatccgattcccctgtagcagtcactgacagttaataaacctttgcaaacgttcaaaaaaaaaaaaaaaaaa

SEQ ID NO: 26 - DNA, 2653 bp, Homo sapiens
ctcaaaaggggccggatttccttctcctggaggcagatgttgcctctctctctcgctcggattggttcagtgcactctagaaacactgctgtggtggagaaactggaccccaggtctggagcgaattccagcctgcagggctgataagcgaggcattagtgagattgagagagactttaccccgccgtggtggttggagggcgcgcagtagagcagcagcacaggcgcgggtcccgggaggccggctctgctcgcgccgagatgtggaatctccttcacgaaaccgactcggctgtggccaccgcgcgccgcccgcgctggctgtgcgctggggcgctggtgctggcgggtggcttctttctcctcggcttcctcttcgggtggtttataaaatcctccaatgaagctactaacattactccaaagcataatatgaaagcatttttggatgaattgaaagctgagaacatcaagaagttcttatataattttacacagataccacatttagcaggaacagaacaaaactttcagcttgcaaagcaaattcaatcccagtggaaagaatttggcctggattctgttgagctagcacattatgatgtcctgttgtcctacccaaataagactcatcccaactacatctcaataattaatgaagatggaaatgagattttcaacacatcattatttgaaccacctcctccaggatatgaaaatgtttcggatattgtaccacctttcagtgctttctctcctcaaggaatgccagagggcgatctagtgtatgttaactatgcacgaactgaagacttctttaaattggaacgggacatgaaaatcaattgctctgggaaaattgtaattgccagatatgggaaagttttcagaggaaataaggttaaaaatgcccagctggcaggggccaaaggagtcattctctactccgaccctgctgactactttgctcctggggtgaagtcctatccagatggttggaatcttcctggaggtggtgtccagcgtggaaatatcctaaatctgaatggtgcaggagaccctctcacaccaggttacccagcaaatgaatatgcttataggcgtggaattgcagaggctgttggtcttccaagtattcctgttcatccaattggatactatgatgcacagaagctcctagaaaaaatgggtggctcagcaccaccagatagcagctggagaggaagtctcaaagtgccctacaatgttggacctggctttactggaaacttttctacacaaaaagtcaagatgcacatccactctaccaatgaagtgacaagaatttacaatgtgataggtactctcagaggagcagtggaaccagacagatatgtcattctgggaggtcaccgggactcatgggtgtttggtggtattgaccctcagagtggagcagctgttgttcatgaaattgtgaggagctttggaacactgaaaaaggaagggtggagacctagaagaacaattttgtttgcaagctgggatgcagaagaatttggtcttcttggttctactgagtgggcagaggagaattcaagactccttcaagagcgtggcgtggcttatattaatgctgactcatctatagaaggaaactacactctgagagttgattgtacaccgctgatgtacagcttggtacacaacctaacaaaagagctgaaaagccctgatgaaggctttgaaggcaaatctctttatgaaagttggactaaaaaaagtccttccccagagttcagtggcatgcccaggataagcaaattgggatctggaaatgattttgaggtgttcttccaacgacttggaattgcttcaggcagagcacggtatactaaaaattgggaaacaaacaaattcagcggctatccactgtatcacagtgtctatgaaacatatgagttggtggaaaagttttatgatccaatgtttaaatatcacctcactgtggcccaggttcgaggagggatggtgtttgagctagccaattccatagtgctcccttttgattgtcgagattatgctgtagttttaagaaagtatgctgacaaaatctacagtatttctatgaaacatccacaggaaatgaagacatacagtgtatcatttgattcacttttttctgcagtaaagaattttacagaaattgcttccaagttcagtgagagactccaggactttgacaaaagcaacccaatagtattaagaatgatgaatgatcaactcatgtttctggaaagagcatttattgatccattagggttaccagacaggcctttttataggcatgtcatctatgctccaagcagccacaacaagtatgcaggggagtcattcccaggaatttatgatgctctgtttgatattgaaagcaaagtggacccttccaaggcctggggagaagtgaagagacagatttatgttgcagccttcacagtgcaggcagctgcagagactttgagtgaagtagcctaagaggattctttagagaatccgtattgaatttgtgtggtatgtcactcagaaagaatcgtaatgggtatattgataaattttaaaattggtatatttgaaataaagttgaatattatatataaaaaaaaaaaaaaaaaaa

SEQ ID NO: 27 - DNA, 2420 bp, Homo sapiens
ggatccaggccctgccaggaaaaatataagggccctgcgtgagaacagagggggtcatccactgcatgagagtggggatgtcacagagtccagcccaccctcctggtagcactgagaagccagggctgtgcttgcggtctgcaccctgagggcccgtggattcctcttcctggagctccaggaaccaggcagtgaggccttggtctgagacagtatcctcaggtcacagagcagaggatgcacagggtgtgccagcagtgaatgtttgccctgaatgcacaccaagggccccacctgccacaggacacataggactccacagagtctggcctcacctccctactgtcagtcctgtagaatcgacctctgctggccggctgtaccctgagtaccctctcacttcctccttcaggttttcaggggacaggccaacccagaggacaggattccctggaggccacagaggagcaccaaggagaagatctgtaagtaggcctttgttagagtctccaaggttcagttctcagctgaggcctctcacacactccctctctccccaggcctgtgggtcttcattgcccagctcctgcccacactcctgcctgctgccctgacgagagtcatcatgtctcttgagcagaggagtctgcactgcaagcctgaggaagcccttgaggcccaacaagaggccctgggcctggtgtgtgtgcaggctgccacctcctcctcctctcctctggtcctgggcaccctggaggaggtgcccactgctgggtcaacagatcctccccagagtcctcagggagcctccgcctttcccactaccatcaacttcactcgacagaggcaacccagtgagggttccagcagccgtgaagaggaggggccaagcacctcttgtatcctggagtccttgttccgagcagtaatcactaagaaggtggctgatttggttggttttctgctcctcaaatatcgagccagggagccagtcacaaaggcagaaatgctggagagtgtcatcaaaaattacaagcactgttttcctgagatcttcggcaaagcctctgagtccttgcagctggtctttggcattgacgtgaaggaagcagaccccaccggccactcctatgtccttgtcacctgcctaggtctctcctatgatggcctgctgggtgataatcagatcatgcccaagacaggcttcctgataattgtcctggtcatgattgcaatggagggcggccatgctcctgaggaggaaatctgggaggagctgagtgtgatggaggtgtatgatgggagggagcacagtgcctatggggagcccaggaagctgctcacccaagatttggtgcaggaaaagtacctggagtaccggcaggtgccggacagtgatcccgcacgctatgagttcctgtggggtccaagggccctcgctgaaaccagctatgtgaaagtccttgagtatgtgatcaaggtcagtgcaagagttcgctttttcttcccatccctgcgtgaagcagctttgagagaggaggaagagggagtctgagcatgagttgcagccaaggccagtgggagggggactgggccagtgcaccttccagggccgcgtccagcagcttcccctgcctcgtgtgacatgaggcccattcttcactctgaagagagcggtcagtgttctcagtagtaggtttctgttctattgggtgacttggagatttatctttgttctcttttggaattgttcaaatgtttttttttaagggatggttgaatgaacttcagcatccaagtttatgaatgacagcagtcacacagttctgtgtatatagtttaagggtaagagtcttgtgttttattcagattgggaaatccattctattttgtgaattgggataataacagcagtggaataagtacttagaaatgtgaaaaatgagcagtaaaatagatgagataaagaactaaagaaattaagagatagtcaattcttgccttatacctcagtctattctgtaaaatttttaaagatatatgcatacctggatttccttggcttctttgagaatgtaagagaaattaaatctgaataaagaattcttcctgttcactggctcttttcttctccatgcactgagcatctgctttttggaaggccctgggttagtagtggagatgctaaggtaagccagactcatacccacccatagggtcgtagagtctaggagctgcagtcacgtaatcgaggtggcaagatgtcctctaaagatgtagggaaaagtgagagaggggtgagggtgtggggctccgggtgagagtggtggagtgtcaatgccctgagctggggcattttgggctttgggaaactgcagttccttctgggggagctgattgtaatgatcttgggtggatcc

SEQ ID NO: 28 - DNA, 4204 bp, Homo sapiens
acgcaggcagtgatgtcacccagaccacaccccttcccccaatgccacttcagggggtactcagagtcagagacttggtctgaggggagcagaagcaatctgcagaggatggcggtccaggctcagccaggcatcaacttcaggaccctgagggatgaccgaaggccccgcccacccacccccaactcccccgaccccaccaggatctacagcctcaggacccccgtcccaatccttaccccttgccccatcaccatcttcatgcttacctccacccccatccgatccccatccaggcagaatccagttccacccctgcccggaacccagggtagtaccgttgccaggatgtgacgccactgacttgcgcattggaggtcagaagaccgcgagattctcgccctgagcaacgagcgacggcctgacgtcggcggagggaagccggcccaggctcggtgaggaggcaaggtaagacgctgagggaggactgaggcgggcctcacctcagacagagggcctcaaataatccagtgctgcctctgctgccgggcctgggccaccccgcaggggaagacttccaggctgggtcgccactacctcaccccgccgacccccgccgctttagccacggggaactctggggacagagcttaatgtggccagggcagggctggttagaagaggtcagggcccacgctgtggcaggaatcaaggtcaggaccccgagagggaactgagggcagcctaaccaccaccctcaccaccattcccgtcccccaacacccaaccccacccccatcccccattcccatccccacccccacccctatcctggcagaatccgggctttgcccctggtatcaagtcacggaagctccgggaatggcggccaggcacgtgagtcctgaggttcacatctacggctaagggagggaaggggttcggtatcgcgagtatggccgttgggaggcagcgaaagggcccaggcctcctggaagacagtggagtcctgaggggacccagcatgccaggacagggggcccactgtacccctgtctcaaaccgaggcaccttttcattcggctacgggaatcctagggatgcagacccacttcagcagggggttggggcccagccctgcgaggagtcatggggaggaagaagagggaggactgaggggaccttggagtccagatcagtggcaaccttgggctgggggatgctgggcacagtggccaaatgtgctctgtgctcattgcgccttcagggtgaccagagagttgagggctgtggtctgaagagtgggacttcaggtcagcagagggaggaatcccaggatctgcagggcccaaggtgtacccccaaggggcccctatgtggtggacagatgcagtggtcctaggatctgccaagcatccaggtgaagagactgagggaggattgagggtacccctgggacagaatgcggactgggggccccataaaaatctgccctgctcctgctgttacctcagagagcctgggcagggctgtcagctgaggtccctccattatcctaggatcactgatgtcagggaaggggaagccttggtctgagggggctgcactcagggcagtagagggaggctctcagaccctactaggagtggaggtgaggaccaagcagtctcctcacccagggtacatggacttcaataaatttggacatctctcgttgtcctttccgggaggacctgggaatgtatggccagatgtgggtcccctcatgtttttctgtaccatatcaggtatgtgagttcttgacatgagagattctcaggccagcagaagggagggattaggccctataaggagaaaggtgagggccctgagtgagcacagaggggatcctccaccccagtagagtggggacctcacagagtctggccaaccctcctgacagttctgggaatccgtggctgcgtttgctgtctgcacattgggggcccgtggattcctctcccaggaatcaggagctccaggaacaaggcagtgaggacttggtctgaggcagtgtcctcaggtcacagagtagagggggctcagatagtgccaacggtgaaggtttgccttggattcaaaccaagggccccacctgccccagaacacatggactccagagcgcctggcctcaccctcaatactttcagtcctgcagcctcagcatgcgctggccggatgtaccctgaggtgccctctcacttcctccttcaggttctgaggggacaggctgacctggaggaccagaggcccccggaggagcactgaaggagaagatctgtaagtaagcctttgttagagcctccaaggttccattcagtactcagctgaggtctctcacatgctccctctctccccaggccagtgggtctccattgcccagctcctgcccacactcccgcctgttgccctgaccagagtcatcatgcctcttgagcagaggagtcagcactgcaagcctgaagaaggccttgaggcccgaggagaggccctgggcctggtgggtgcgcaggctcctgctactgaggagcaggaggctgcctcctcctcttctactctagttgaagtcaccctgggggaggtgcctgctgccgagtcaccagatcctccccagagtcctcagggagcctccagcctccccactaccatgaactaccctctctggagccaatcctatgaggactccagcaaccaagaagaggaggggccaagcaccttccctgacctggagtccgagttccaagcagcactcagtaggaaggtggccgagttggttcattttctgctcctcaagtatcgagccagggagccggtcacaaaggcagaaatgctggggagtgtcgtcggaaattggcagtatttctttcctgtgatcttcagcaaagcttccagttccttgcagctggtctttggcatcgagctgatggaagtggaccccatcggccacttgtacatctttgccacctgcctgggcctctcctacgatggcctgctgggtgacaatcagatcatgcccaaggcaggcctcctgataatcgtcctggccataatcgcaagagagggcgactgtgcccctgaggagaaaatctgggaggagctgagtgtgttagaggtgtttgaggggagggaagacagtatcttgggggatcccaagaagctgctcacccaacatttcgtgcaggaaaactacctggagtaccggcaggtccccggcagtgatcctgcatgttatgaattcctgtggggtccaagggccctcgttgaaaccagctatgtgaaagtcctgcaccatatggtaaagatcagtggaggacctcacatttcctacccacccctgcatgagtgggttttgagagagggggaagagtgagtctgagcacgagttgcagccagggccagtgggagggggtctgggccagtgcaccttccggggccgcatcccttagtttccactgcctcctgtgacgtgaggcccattcttcactctttgaagcgagcagtcagcattcttagtagtgggtttctgttctgttggatgactttgagattattctttgtttcctgttggagttgttcaaatgttccttttaacggatggttgaatgagcgtcagcatccaggtttatgaatgacagtagtcacacatagtgctgtttatatagtttaggagtaagagtcttgttttttactcaaattgggaaatccattccattttgtgaattgtgacataataatagcagtggtaaaagtatttgcttaaaattgtgagcgaattagcaataacatacatgagataactcaagaaatcaaaagatagttgattcttgccttgtacctcaatctattctgtaaaattaaacaaatatgcaaaccaggatttccttgacttctttgagaatgcaagcgaaattaaatctgaataaataattcttcctcttcactggctcgtttcttttccgttcactcagcatctgctctgtgggaggccctgggttagtagtggggatgctaaggtaagccagactcacgcctacccatagggctgtagagcctaggacctgcagtcatataattaaggtggtgagaagtcctgtaagatgtagaggaaatgtaagagaggggtgagggtgtggcgctccgggtgagagtagtggagtgtcagtgc

SEQ ID NO: 29 - DNA, 752 bp, Homo sapiens
atcctcgtgggccctgaccttctctctgagagccgggcagaggctccggagccatgcaggccgaaggccggggcacagggggttcgacgggcgatgctgatggcccaggaggccctggcattcctgatggcccagggggcaatgctggcggcccaggagaggcgggtgccacgggcggcagaggtccccggggcgcaggggcagcaagggcctcggggccgggaggaggcgccccgcggggtccgcatggcggcgcggcttcagggctgaatggatgctgcagatgcggggccagggggccggagagccgcctgcttgagttctacctcgccatgcctttcgcgacacccatggaagcagagctggcccgcaggagcctggcccaggatgccccaccgcttcccgtgccaggggtgcttctgaaggagttcactgtgtccggcaacatactgactatccgactgactgctgcagaccaccgccaactgcagctctccatcagctcctgtctccagcagctttccctgttgatgtggatcacgcagtgctttctgcccgtgtttttggctcagcctccctcagggcagaggcgctaagcccagcctggcgccccttcctaggtcatgcctcctcccctagggaatggtcccagcacgagtggccagttcattgtgggggcctgattgtttgtcgctggaggaggacggcttacatgtttgtttctgtagaaaataaaactgagctacgaaaaa

SEQ ID NO: 30 - DNA, 2148 bp, Homo sapiens
gcttcagggtacagctcccccgcagccagaagccgggcctgcagcgcctcagcaccgctccgggacaccccacccgcttcccaggcgtgacctgtcaacagcaacttcgcggtgtggtgaactctctgaggaaaaaccattttgattattactctcagacgtgcgtggcaacaagtgactgagacctagaaatccaagcgttggaggtcctgaggccagcctaagtcgcttcaaaatggaacgaaggcgtttgtggggttccattcagagccgatacatcagcatgagtgtgtggacaagcccacggagacttgtggagctggcagggcagagcctgctgaaggatgaggccctggccattgccgccctggagttgctgcccagggagctcttcccgccactcttcatggcagcctttgacgggagacacagccagaccctgaaggcaatggtgcaggcctggcccttcacctgcctccctctgggagtgctgatgaagggacaacatcttcacctggagaccttcaaagctgtgcttgatggacttgatgtgctccttgcccaggaggttcgccccaggaggtggaaacttcaagtgctggatttacggaagaactctcatcaggacttctggactgtatggtctggaaacagggccagtctgtactcatttccagagccagaagcagctcagcccatgacaaagaagcgaaaagtagatggtttgagcacagaggcagagcagcccttcattccagtagaggtgctcgtagacctgttcctcaaggaaggtgcctgtgatgaattgttctcctacctcattgagaaagtgaagcgaaagaaaaatgtactacgcctgtgctgtaagaagctgaagatttttgcaatgcccatgcaggatatcaagatgatcctgaaaatggtgcagctggactctattgaagatttggaagtgacttgtacctggaagctacccaccttggcgaaattttctccttacctgggccagatgattaatctgcgtagactcctcctctcccacatccatgcatcttcctacatttccccggagaaggaagagcagtatatcgcccagttcacctctcagttcctcagtctgcagtgcctgcaggctctctatgtggactctttatttttccttagaggccgcctggatcagttgctcaggcacgtgatgaaccccttggaaaccctctcaataactaactgccggctttcggaaggggatgtgatgcatctgtcccagagtcccagcgtcagtcagctaagtgtcctgagtctaagtggggtcatgctgaccgatgtaagtcccgagcccctccaagctctgctggagagagcctctgccaccctccaggacctggtctttgatgagtgtgggatcacggatgatcagctccttgccctcctgccttccctgagccactgctcccagcttacaaccttaagcttctacgggaattccatctccatatctgccttgcagagtctcctgcagcacctcatcgggctgagcaatctgacccacgtgctgtatcctgtccccctggagagttatgaggacatccatggtaccctccacctggagaggcttgcctatctgcatgccaggctcagggagttgctgtgtgagttggggcggcccagcatggtctggcttagtgccaacccctgtcctcactgtggggacagaaccttctatgacccggagcccatcctgtgcccctgtttcatgcctaactagctgggtgcacatatcaaatgcttcattctgcatacttggacactaaagccaggatgtgcatgcatcttgaagcaacaaagcagccacagtttcagacaaatgttcagtgtgagtgaggaaaacatgttcagtgaggaaaaaacattcagacaaatgttcagtgaggaaaaaaaggggaagttggggataggcagatgttgacttgaggagttaatgtgatctttggggagatacatcttatagagttagaaatagaatctgaatttctaaagggagattctggcttgggaagtacatgtaggagttaatccctgtgtagactgttgtaaagaaactgttgaaaataaagagaagcaatgtgaagcaaaaaaaaaaaaaaaaaa

SEQ ID NO: 31 - DNA, 4530 bp, Homo sapiens
aattctcgagctcgtcgaccggtcgacgagctcgagggtcgacgagctcgagggcgcgcgcccggcccccacccctcgcagcaccccgcgccccgcgccctcccagccgggtccagccggagccatggggccggagccgcagtgagcaccatggagctggcggccttgtgccgctgggggctcctcctcgccctcttgccccccggagccgcgagcacccaagtgtgcaccggcacagacatgaagctgcggctccctgccagtcccgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaaggacatcttccacaagaacaaccagctggctctcacactgatagacaccaaccgctctcgggcctgccacccctgttctccgatgtgtaagggctcccgctgctggggagagagttctgaggattgtcagagcctgacgcgcactgtctgtgccggtggctgtgcccgctgcaaggggccactgcccactgactgctgccatgagcagtgtgctgccggctgcacgggccccaagcactctgactgcctggcctgcctccacttcaaccacagtggcatctgtgagctgcactgcccagccctggtcacctacaacacagacacgtttgagtccatgcccaatcccgagggccggtatacattcggcgccagctgtgtgactgcctgtccctacaactacctttctacggacgtgggatcctgcaccctcgtctgccccctgcacaaccaagaggtgacagcagaggatggaacacagcggtgtgagaagtgcagcaagccctgtgcccgagtgtgctatggtctgggcatggagcacttgcgagaggtgagggcagttaccagtgccaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcactgctggggtccagggcccacccagtgtgtcaactgcagccagttccttcggggccaggagtgcgtggaggaatgccgagtactgcaggggctccccagggagtatgtgaatgccaggcactgtttgccgtgccaccctgagtgtcagccccagaatggctcagtgacctgttttggaccggaggctgaccagtgtgtggcctgtgcccactataaggaccctcccttctgcgtggcccgctgccccagcggtgtgaaacctgacctctcctacatgcccatctggaagtttccagatgaggagggcgcatgccagccttgccccatcaactgcacccactcctgtgtggacctggatgacaagggctgccccgccgagcagagagccagccctctgacgtccatcgtctctgcggtggttggcattctgctggtcgtggtcttgggggtggtctttgggatcctcatcaagcgacggcagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagaccatgtccgggaaaaccgcggacgcctgggctcccaggacctgctgaactggtgtatgcagattgccaaggggatgagctacctggaggatgtgcggctcgtacacagggacttggccgctcggaacgtgctggtcaagagtcccaaccatgtcaaaattacagacttcgggctggctcggctgctggacattgacgagacagagtaccatgcagatgggggcaaggtgcccatcaagtggatggcgctggagtccattctccgccggcggttcacccaccagagtgatgtgtggagttatggtgtgactgtgtgggagctgatgacttttggggccaaaccttacgatgggatcccagcccgggagatccctgacctgctggaaaagggggagcggctgccccagccccccatctgcaccattgatgtctacatgatcatggtcaaatgttggatgattgactctgaatgtcggccaagattccgggagttggtgtctgaattctcccgcatggccagggacccccagcgctttgtggtcatccagaatgaggacttgggcccagccagtcccttggacagcaccttctaccgctcactgctggaggacgatgacatgggggacctggtggatgctgaggagtatctggtaccccagcagggcttcttctgtccagaccctgccccgggcgctgggggcatggtccaccacaggcaccgcagctcatctaccaggagtggcggtggggacctgacactagggctggagccctctgaagaggaggcccccaggtctccactggcaccctccgaaggggctggctccgatgtatttgatggtgacctgggaatgggggcagccaaggggctgcaaagcctccccacacatgaccccagccctctacagcggtacagtgaggaccccacagtacccctgccctctgagactgatggctacgttgcccccctgacctgcagcccccagcctgaatatgtgaaccagccagatgttcggccccagcccccttcgccccgagagggccctctgcctgctgcccgacctgctggtgccactctggaaagggccaagactctctccccagggaagaatggggtcgtcaaagacgtttttgcctttgggggtgccgtggagaaccccgagtacttgacaccccagggaggagctgcccctcagccccaccctcctcctgccttcagcccagccttcgacaacctctattactgggaccaggacccaccagagcggggggctccacccagcaccttcaaagggacacctacggcagagaacccagagtacctgggtctggacgtgccagtgtgaaccagaaggccaagtccgcagaagccctgatgtgtcctcagggagcagggaaggcctgacttctgctggcatcaagaggtgggagggccctccgaccacttccaggggaacctgccatgccaggaacctgtcctaaggaaccttccttcctgcttgagttcccagatggctggaaggggtccagcctcgttggaagaggaacagcactggggagtctttgtggattctgaggccctgcccaatgagactctagggtccagtggatgccacagcccagcttggccctttccttccagatcctgggtactgaaagccttagggaagctggcctgagaggggaagcggccctaagggagtgtctaagaacaaaagcgacccattcagagactgtccctgaaacctagtactgccccccatgaggaaggaacagcaatggtgtcagtatccaggctttgtacagagtgcttttctgtttagtttttactttttttgttttgtttttttaaagacgaaataaagacccaggggagaatgggtgttgtatggggaggcaagtgtggggggtccttctccacacccactttgtccatttgcaaatatattttggaaaac

SEQ ID NO: 32 - DNA, 1464 bp, Homo sapiens
agccccaagcttaccacctgcacccggagagctgtgtcaccatgtgggtcccggttgtcttcctcaccctgtccgtgacgtggattggtgctgcacccctcatcctgtctcggattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgtaatggtgtgcttcaaggtatcacgtcatggggcagtgaaccatgtgccctgcccgaaaggccttccctgtacaccaaggtggtgcattaccggaagtggatcaaggacaccatcgtggccaacccctgagcacccctatcaaccccctattgtagtaaacttggaaccttggaaatgaccaggccaagactcaagcctccccagttctactgacctttgtccttaggtgtgaggtccagggttgctaggaaaagaaatcagcagacacaggtgtagaccagagtgtttcttaaatggtgtaattttgtcctctctgtgtcctggggaatactggccatgcctggagacatatcactcaatttctctgaggacacagataggatggggtgtctgtgttatttgtggggtacagagatgaaagaggggtgggatccacactgagagagtggagagtgacatgtgctggacactgtccatgaagcactgagcagaagctggaggcacaacgcaccagacactcacagcaaggatggagctgaaaacataacccactctgtcctggaggcactgggaagcctagagaaggctgtgagccaaggagggagggtcttcctttggcatgggatggggatgaagtaaggagagggactggaccccctggaagctgattcactatggggggaggtgtattgaagtcctccagacaaccctcagatttgatgatttcctagtagaactcacagaaataaagagctgttatactgtg

SEQ ID NO: 33 - DNA, 781 bp, Homo sapiens
gtcacacccggaagcaggggcccgagcggagccggccgcgatgagcggggagccggggcagacgtccgtagcgccccctcccgaggaggtcgagccgggcagtggggtccgcatcgtggtggagtactgtgaaccctgcggcttcgaggcgacctacctggagctggccagtgctgtgaaggagcagtatccgggcatcgagatcgagtcgcgcctcgggggcacaggtgcctttgagatagagataaatggacagctggtgttctccaagctggagaatgggggctttccctatgagaaagatctcattgaggccatccgaagagccagtaatggagaaaccctagaaaagatcaccaacagccgtcctccctgcgtcatcctgtgactgcacaggactctgggttcctgctctgttctggggtccaaaccttggtctccctttggtcctgctgggagctccccctgcctctttcccctacttagctccttagcaaagagaccctggcctccactttgccctttgggtacaaagaaggaatagaagattccgtggccttgggggcaggagagagacactctccatgaacacttctccagccacctcatacccccttcccagggtaagtgcccacgaaagcccagtccactcttcgcctcggtaatacctgtctgatgccacagattttatttattctcccctaacccagggcaatgtcagctattggcagtaaagtggcgctacaaacactaaaaaaaaaaaaaaaaaaaaaaaaaaaaa

SEQ ID NO: 34 - DNA, 1114 bp, Homo sapiens
acacgccgatttgcccttttgattcttccacaatcagggtgagactgctcccagtgccatgaacggagacgacgcctttgcaaggagacccagggatgatgctcaaatatcagagaagttacgaaaggccttcgatgatattgccaaatacttctctaagaaagagtgggaaaagatgaaatcctcggagaaaatcgtctatgtgtatatgaagctaaactatgaggtcatgactaaactaggtttcaaggtcaccctcccacctttcatgcgtagtaaacgggctgcagacttccacgggaatgattttggtaacgatcgaaaccacaggaatcaggttgaacgtcctcagatgactttcggcagcctccagagaatcttcccgaaggacccaaaagggggaaacatgcctggacccacagactgcgtgagagaaagcagctggtggtttatgaagagatcagcgaccctgaggaagatgacgagtaactcccctcggggatatgacacatgcccatgatgagaagcagaacgtggtgacctttcacgaacatgggcatggctgcggacccctcgtcatcaggtgcatagcaagtgaaagcaagtgttcacaacagtgaaaagttgagcgtcatttttcttagtgtgccaagagttcgatgttggcgtttccgctgtattttcttgcagtgtgccattctgttagacattagcgttttcgttgatgagcaagacatgcttaatgcatatttcggcttgtgtatccatgcacctacctcagaaaacaagtattgtcaggtattctctccatagaacagcactaccctcctctctccccagatgtgactactgaggggaggtctgagtgtttaatttccgattttttcctctgcatttacacacacaccacacacgcacacacacacaccaagtaccagtataagcatctcccatctgcttttctccattgccatgcgacctggtcaagcccccctcactctgtttcctgttcagcatgtactcccctcatccgattccgttgtatcagtcactgacagttaataaacctttgcaaacgttcaaaaaaaaaaaaaaaaaaaaaaaaaaaaaaaaa

SEQ ID NO: 35 - DNA, 2143 bp, Homo sapiens
agtgcctttggttgctggagggaagaacacaatggatctggtgctaaaaagatgccttcttcatttggctgtgataggtgctttgctggctgtgggggctacaaaagtacccagaaaccaggactggcttggtgtctcaaggcaactcagaaccaaagcctggaacaggcagctgtatccagagtggacagaagcccagagacttgactgctggagaggtggtcaagtgtccctcaaggtcagtaatgatgggcctacactgattggtgcaaatgcctccttctctattgccttgaacttccctggaagccaaaaggtattgccagatgggcaggttatctgggtcaacaataccatcatcaatgggagccaggtgtggggaggacagccagtgtatccccaggaaactgacgatgcctgcatcttccctgatggtggaccttgcccatctggctcttggtctcagaagagaagctttgtttatgtctggaagacctggggccaatactggcaagttctagggggcccagtgtctgggctgagcattgggacaggcagggcaatgctgggcacacacaccatggaagtgactgtctaccatcgccggggatcccggagctatgtgcctcttgctcattccagctcagccttcaccattactgaccaggtgcctttctccgtgagcgtgtcccagttgcgggccttggatggagggaacaagcacttcctgagaaatcagcctctgacctttgccctccagctccatgaccccagtggctatctggctgaagctgacctctcctacacctgggactttggagacagtagtggaaccctgatctctcgggcacttgtggtcactcatacttacctggagcctggcccagtcactgcccaggtggtcctgcaggctgccattcctctcacctcctgtggctcctccccagttccaggcaccacagatgggcacaggccaactgcagaggcccctaacaccacagctggccaagtgcctactacagaagttgtgggtactacacctggtcaggcgccaactgcagagccctctggaaccacatctgtgcaggtgccaaccactgaagtcataagcactgcacctgtgcagatgccaactgcagagagcacaggtatgacacctgagaaggtgccagtttcagaggtcatgggtaccacactggcagagatgtcaactccagaggctacaggtatgacacctgcagaggtatcaattgtggtgctttctggaaccacagctgcacaggtaacaactacagagtgggtggagaccacagctagagagctacctatccctgagcctgaaggtccagatgccagctcaatcatgtctacggaaagtattacaggttccctgggccccctgctggatggtacagccaccttaaggctggtgaagagacaagtccccctggattgtgttctgtatcgatatggttccttttccgtcaccctggacattgtccagggtattgaaagtgccgagatcctgcaggctgtgccgtccggtgagggggatgcatttgagctgactgtgtcctgccaaggcgggctgcccaaggaagcctgcatggagatctcatcgccagggtgccagccccctgcccagcggctgtgccagcctgtgctacccagcccagcctgccagctggttctgcaccagatactgaagggtggctcggggacatactgcctcaatgtgtctctggctgataccaacagcctggcagtggtcagcacccagcttatcatgcctggtcaagaagcaggccttgggcaggttccgctgatcgtgggcatcttgctggtgttgatggctgtggtccttgcatctctgatatataggcgcagacttatgaagcaagacttctccgtaccccagttgccacatagcagcagtcactggctgcgtctaccccgcatcttctgctcttgtcccattggtgagaatagccccctcctcagtgggcagcaggtctgagtactctcatatgatgctgtgattttcctggagttgacagaaacacctatatttcccccagtcttccctgggagactactattaactgaaataaatactcagagcctgaaaaaaaaaaaaaaaaaa

SEQ ID NO: 36 - DNA, 2352 bp, Homo sapiens
gaaacttaaaggtgtttaccttgtcatcagcatgtaagctaattatctcgggcaagatgtaggcttctattgtcttgttgctttagcgcttacgccccgcctctggtggctgcctaaaacctggcgccgggctaaaacaaacgcgaggcagcccccgagcctccactcaagccaattaaggaggactcggtccactccgttacgtgtacatccaacaagatcggcgttaaggtaacaccagaatatttggcaaagggagaaaaaaaaagcagcgaggcttcgccttccccctctcccttttttttcctcctcttccttcctcctccagccgccgccgaatcatgtcgatgagtccaaagcacacgactccgttctcagtgtctgacatcttgagtcccctggaggaaagctacaagaaagtgggcatggagggcggcggcctcggggctccgctggcggcgtacaggcagggccaggcggcaccgccaacagcggccatgcagcagcacgccgtggggcaccacggcgccgtcaccgccgcctaccacatgacggcggcgggggtgccccagctctcgcactccgccgtggggggctactgcaacggcaacctgggcaacatgagcgagctgccgccgtaccaggacaccatgaggaacagcgcctctggccccggatggtacggcgccaacccagacccgcgcttccccgccatctcccgcttcatgggcccggcgagcggcatgaacatgagcggcatgggcggcctgggctcgctgggggacgtgagcaagaacatggccccgctgccaagcgcgccgcgcaggaagcgccgggtgctcttctcgcaggcgcaggtgtacgagctggagcgacgcttcaagcaacagaagtacctgtcggcgccggagcgcgagcacctggccagcatgatccacctgacgcccacgcaggtcaagatctggttccagaaccaccgctacaaaatgaagcgccaggccaaggacaaggcggcgcagcagcaactgcagcaggacagcggcggcggcgggggcggcgggggcaccgggtgcccgcagcagcaacaggctcagcagcagtcgccgcgacgcgtggcggtgccggtcctggtgaaagacggcaaaccgtgccaggcgggtgcccccgcgccgggcgccgccagcctacaaggccacgcgcagcagcaggcgcagcaccaggcgcaggccgcgcaggcggcggcagcggccatctccgtgggcagcggtggcgccggccttggcgcacacccgggccaccagccaggcagcgcaggccagtctccggacctggcgcaccacgccgccagccccgcggcgctgcagggccaggtatccagcctgtcccacctgaactcctcgggctcggactacggcaccatgtcctgctccaccttgctatacggtcggacctggtgagaggacgccgggccggccctagcccagcgctctgcctcaccgcttccctcctgcccgccacacagaccaccatccaccgctgctccacgcgcttcgacttttcttaacaacctggccgcgtttagaccaaggaacaaaaaaaccacaaaggccaaactgctggacgtctttctttttttccccccctaaaatttgtgggtttttttttttaaaaaaagaaaatgaaaaacaaccaagcgcatccaatctcaaggaatctttaagcagagaagggcataaaacagctttggggtgtctttttttggtgattcaaatgggttttccacgctagggcggggcacagattggagagggctctgtgctgacatggctctggactctaaagaccaaacttcactctgggcacactctgccagcaaagaggactcgcttgtaaataccaggatttttttttttttttgaagggaggacgggagctggggagaggaaagagtcttcaacataacccacttgtcactgacacaaaggaagtgccccctccccggcaccctctggccgcctaggctcagcggcgaccgccctccgcgaaaatagtttgtttaatgtgaacttgtagctgtaaaacgctgtcaaaagttggactaaatgcctagtttttagtaatctgtacattttgttgtaaaaagaaaaaccactcccagtccccagcccttcacattttttatgggcattgacaaatctgtgtatattatttggcagtttggtatttgcggcgtcagtctttttctgttgtaacttatgtagatatttggcttaaatatagttcctaagaagcttctaataaattatacaaattaaaaagattctttttctgattaaaaaaaaaaaaaaaaaa

SEQ ID NO: 37 - DNA, 503 bp, Homo sapiens
gacagcggcttccttgatccttgccacccgcgactgaacaccgacagcagcagcctcaccatgaagttgctgatggtcctcatgctggcggccctctcccagcactgctacgcaggctctggctgccccttattggagaatgtgatttccaagacaatcaatccacaagtgtctaagactgaatacaaagaacttcttcaagagttcatagacgacaatgccactacaaatgccatagatgaattgaaggaatgttttcttaaccaaacggatgaaactctgagcaatgttgaggtgtttatgcaattaatatatgacagcagtctttgtgatttattttaactttctgcaagacctttggctcacagaactgcagggtatggtgagaaaccaactacggattgctgcaaaccacaccttctctttcttatgtctttttactacaaactacaagacaattgttgaaacctgctatacatgtttattttaataaattgatggca

SEQ ID NO: 38 - DNA, 591 bp, Homo sapiens
cttctctgggacacattgccttctgttttctccagcatgcgcttgctccagctcctgttcagggccagccctgccaccctgctcctggttctctgcctgcagttgggggccaacaaagctcaggacaacactcggaagatcataataaagaattttgacattcccaagtcagtacgtccaaatgacgaagtcactgcagtgcttgcagttcaaacagaattgaaagaatgcatggtggttaaaacttacctcattagcagcatccctctacaaggtgcatttaactataagtatactgcctgcctatgtgacgacaatccaaaaaccttctactgggacttttacaccaacagaactgtgcaaattgcagccgtcgttgatgttattcgggaattaggcatctgccctgatgatgctgctgtaatccccatcaaaaacaaccggttttatactattgaaatcctaaaggtagaataatggaagccctgtctgtttgccacacccaggtgatttcctctaaagaaacttggctggaatttctgctgtggtctataaaataaacttcttaacatgcttaaaaaaaaaaaaaaa

SEQ ID NO: 39 - DNA, 2388 bp, Homo sapiens
ggaaaccgaggcagaggaggctcaggtgtggccaatcaccctgcacatcagagttaccctgggcagggcccactgagacctgggaggggccactcgggacctggagggctgggggctgcccgggcgttaggggtaaagctccctacccaactgcgcagaaggcctcagaggcctgggggctgggcttcccctttcacatcgccctttagaggcccacgtgtgggcattggcccgcgatctgaaaggggctgtcctgttcctcatgggcgctgccagcgccacgcactcctctttctgcctggccggccactcccgtctgctgtgacgcgcggacagagagctaccggtggacccacggtgcctccctccctgggatctacacagaccatggccttgccaacggctcgacccctgttggggtcctgtgggacccccgccctcggcagcctcctgttcctgctcttcagcctcggatgggtgcagccctcgaggaccctggctggagagacagggcaggaggctgcgcccctggacggagtcctggccaacccacctaacatttccagcctctcccctcgccaactccttggcttcccgtgtgcggaggtgtccggcctgagcacggagcgtgtccgggagctggctgtggccttggcacagaagaatgtcaagctctcaacagagcagctgcgctgtctggctcaccggctctctgagccccccgaggacctggacgccctcccattggacctgctgctattcctcaacccagatgcgttctcggggccccaggcctgcacccgtttcttctcccgcatcacgaaggccaatgtggacctgctcccgaggggggctcccgagcgacagcggctgctgcctgcggctctggcctgctggggtgtgcgggggtctctgctgagcgaggctgatgtgcgggctctgggaggcctggcttgcgacctgcctgggcgctttgtggccgagtcggccgaagtgctgctaccccggctggtgagctgcccgggacccctggaccaggaccagcaggaggcagccagggcggctctgcagggcgggggacccccctacggccccccgtcgacatggtctgtctccacgatggacgctctgcggggcctgctgcccgtgctgggccagcccatcatccgcagcatcccgcagggcatcgtggccgcgtggcggcaacgctcctctcgggacccatcctggcggcagcctgaacggaccatcctccggccgcggttccggcgggaagtggagaagacagcctgtccttcaggcaagaaggcccgcgagatagacgagagcctcatcttctacaagaagtgggagctggaagcctgcgtggatgcggccctgctggccacccagatggaccgcgtgaacgccatccccttcacctacgagcagctggacgtcctaaagcataaactggatgagctctacccacaaggttaccccgagtctgtgatccagcacctgggctacctcttcctcaagatgagccctgaggacattcgcaagtggaatgtgacgtccctggagaccctgaaggctttgcttgaagtcaacaaagggcacgaaatgagtcctcaggtggccaccctgatcgaccgctttgtgaagggaaggggccagctagacaaagacaccctagacaccctgaccgccttctaccctgggtacctgtgctccctcagccccgaggagctgagctccgtgccccccagcagcatctgggcggtcaggccccaggacctggacacgtgtgacccaaggcagctggacgtcctctatcccaaggcccgccttgctttccagaacatgaacgggtccgaatacttcgtgaagatccagtccttcctgggtggggcccccacggaggatttgaaggcgctcagtcagcagaatgtgagcatggacttggccacgttcatgaagctgcggacggatgcggtgctgccgttgactgtggctgaggtgcagaaacttctgggaccccacgtggagggcctgaaggcggaggagcggcaccgcccggtgcgggactggatcctacggcagcggcaggacgacctggacacgctggggctggggctacagggcggcatccccaacggctacctggtcctagacctcagcatgcaagaggccctctcggggacgccctgcctcctaggacctggacctgttctcaccgtcctggcactgctcctagcctccaccctggcctgagggccccactcccttgctggccccagccctgctggggatccccgcctggccaggagcaggcacgggtggtccccgttccaccccaagagaactcgcgctcagtaaacgggaacatgccccctgcagacacgt

SEQ ID NO: 40 - DNA, 2412 bp, Homo sapiens
ggaaaccgaggcagaggaggctcaggtgtggccaatcaccctgcacatcagagttaccctgggcagggcccactgagacctgggaggggccactcgggacctggagggctgggggctgcccgggcgttaggggtaaagctccctacccaactgcgcagaaggcctcagaggcctgggggctgggcttcccctttcacatcgccctttagaggcccacgtgtgggcattggcccgcgatctgaaaggggctgtcctgttcctcatgggcgctgccagcgccacgcactcctctttctgcctggccggccactcccgtctgctgtgacgcgcggacagagagctaccggtggacccacggtgcctccctccctgggatctacacagaccatggccttgccaacggctcgacccctgttggggtcctgtgggacccccgccctcggcagcctcctgttcctgctcttcagcctcggatgggtgcagccctcgaggaccctggctggagagacagggcaggaggctgcgcccctggacggagtcctggccaacccacctaacatttccagcctctcccctcgccaactccttggcttcccgtgtgcggaggtgtccggcctgagcacggagcgtgtccgggagctggctgtggccttggcacagaagaatgtcaagctctcaacagagcagctgcgctgtctggctcaccggctctctgagccccccgaggacctggacgccctcccattggacctgctgctattcctcaacccagatgcgttctcggggccccaggcctgcacccgtttcttctcccgcatcacgaaggccaatgtggacctgctcccgaggggggctcccgagcgacagcggctgctgcctgcggctctggcctgctggggtgtgcgggggtctctgctgagcgaggctgatgtgcgggctctgggaggcctggcttgcgacctgcctgggcgctttgtggccgagtcggccgaagtgctgctaccccggctggtgagctgcccgggacccctggaccaggaccagcaggaggcagccagggcggctctgcagggcgggggacccccctacggccccccgtcgacatggtctgtctccacgatggacgctctgcggggcctgctgcccgtgctgggccagcccatcatccgcagcatcccgcagggcatcgtggccgcgtggcggcaacgctcctctcgggacccatcctggcggcagcctgaacggaccatcctccggccgcggttccggcgggaagtggagaagacagcctgtccttcaggcaagaaggcccgcgagatagacgagagcctcatcttctacaagaagtgggagctggaagcctgcgtggatgcggccctgctggccacccagatggaccgcgtgaacgccatccccttcacctacgagcagctggacgtcctaaagcataaactggatgagctctacccacaaggttaccccgagtctgtgatccagcacctgggctacctcttcctcaagatgagccctgaggacattcgcaagtggaatgtgacgtccctggagaccctgaaggctttgcttgaagtcaacaaagggcacgaaatgagtcctcaggctcctcggcggcccctcccacaggtggccaccctgatcgaccgctttgtgaagggaaggggccagctagacaaagacaccctagacaccctgaccgccttctaccctgggtacctgtgctccctcagccccgaggagctgagctccgtgccccccagcagcatctgggcggtcaggccccaggacctggacacgtgtgacccaaggcagctggacgtcctctatcccaaggcccgccttgctttccagaacatgaacgggtccgaatacttcgtgaagatccagtccttcctgggtggggcccccacggaggatttgaaggcgctcagtcagcagaatgtgagcatggacttggccacgttcatgaagctgcggacggatgcggtgctgccgttgactgtggctgaggtgcagaaacttctgggaccccacgtggagggcctgaaggcggaggagcggcaccgcccggtgcgggactggatcctacggcagcggcaggacgacctggacacgctggggctggggctacagggcggcatccccaacggctacctggtcctagacctcagcatgcaagaggccctctcggggacgccctgcctcctaggacctggacctgttctcaccgtcctggcactgctcctagcctccaccctggcctgagggccccactcccttgctggccccagccctgctggggatccccgcctggccaggagcaggcacgggtggtccccgttccaccccaagagaactcgcgctcagtaaacgggaacatgccccctgcagacacgt

SEQ ID NO: 41 - DNA, 946 bp, Homo sapiens
tgacagcccaccagtgaccatgaaggctgtgctgcttgccctgttgatggcaggcttggccctgcagccaggcactgccctgctgtgctactcctgcaaagcccaggtgagcaacgaggactgcctgcaggtggagaactgcacccagctgggggagcagtgctggaccgcgcgcatccgcgcagttggcctcctgaccgtcatcagcaaaggctgcagcttgaactgcgtggatgactcacaggactactacgtgggcaagaagaacatcacgtgctgtgacaccgacttgtgcaacgccagcggggcccatgccctgcagccggctgccgccatccttgcgctgctccctgcactcggcctgctgctctggggacccggccagctataggctctggggggccccgctgcagcccacactgggtgtggtgccccaggcctctgtgccactcctcacagacctggcccagtgggagcctgtcctggttcctgaggcacatcctaacgcaagtctgaccatgtatgtttgcacccctgtcccccaccctgaccctcccatggccctctccaggactcccacccggcagatcagctctagtgacacagatccgcctgcagatggcccctccaaccctctctgctgctgtttccatggcccagcattctccacccttaaccctgtgctcaggcacctcttcccccaggaagccttccctgcccaccccatctatgacttgagccaggtctggtccgtggtgtcccccgcacccagcaggggacaggcactcaggagggcccagtaaaggctgagatgaagtggactgagtagaactggaggacaagagtcgacgtgagttcctgggagtctccagagatggggcctggaggcctggaggaaggggccaggcctcacattcgtggggctccctgaatggcagcctgagca

SEQ ID NO: 42 - DNA, 3503 bp, Homo sapiens
ttatttagccgttgaactgccagccgcccggggcccacgcgctccgggccgctcaggctgagcgatttcccgccttttctgaggttctgaggcgggagccattggttctttctgttgccctcatagaccgtatgtagcagttcgcgtgggcacagaacccacggtttcccgctagttcttcaaagatatttacaaccgtaacagagaaaatggaaaagcaaaagccctttgcattgttcgtaccaccgagatcaagcagcagtcaggtgtctgcggtgaaacctcagaccctgggaggcgattccactttcttcaagagtttcaacaaatgtactgaagatgattttgagtttccatttgcaaagactaatctctccaaaaatggggaaaacattgattcagatcctgctttacaaaaagttaatttcttgcccgtgcttgagcaggttggtaattctgactgtcactatcaggaaggactaaaagactctgatttggagaattcagagggattgagcagagtgtattcaaaactgtataaggaggctgaaaagataaaaaaatggaaagtaagtacagaagctgaactgagacagaaagaaagtaagttgcaagaaaacagaaagataattgaagcacagcgaaaagccattcaggaactgcaatttggaaatgaaaaagtaagtttgaaattagaagaaggaatacaagaaaataaagatttaataaaagagaataatgccacaaggcatttatgtaatctactcaaagaaacctgtgctagatctgcagaaaagacaaagaaatatgaatatgaacgggaagaaaccaggcaagtttatatggatctaaataataacattgagaaaatgataacagcttttgaggaacttcgtgtgcaagctgagaattccagactggaaatgcattttaagttaaaggaagattatgaaaaaatccaacaccttgaacaagaatacaagaaggaaataaatgacaaggaaaagcaggtatcactactattgatccaaatcactgagaaagaaaataaaatgaaagatttaacatttctgctagaggaatccagagataaagttaatcaattagaggaaaagacaaaattacagagtgaaaacttaaaacaatcaattgagaaacagcatcatttgactaaagaactagaagatattaaagtgtcattacaaagaagtgtgagtactcaaaaggctttagaggaagatttacagatagcaacaaaaacaatttgtcagctaactgaagaaaaagaaactcaaatggaagaatctaataaagctagagctgctcattcgtttgtggttactgaatttgaaactactgtctgcagcttggaagaattattgagaacagaacagcaaagattggaaaaaaatgaagatcaattgaaaatacttaccatggagcttcaaaagaaatcaagtgagctggaagagatgactaagcttacaaataacaaagaagtagaacttgaagaattgaaaaaagtcttgggagaaaaggaaacacttttatatgaaaataaacaatttgagaagattgctgaagaattaaaaggaacagaacaagaactaattggtcttctccaagccagagagaaagaagtacatgatttggaaatacagttaactgccattaccacaagtgaacagtattattcaaaagaggttaaagatctaaaaactgagcttgaaaacgagaagcttaagaatactgaattaacttcacactgcaacaagctttcactagaaaacaaagagctcacacaggaaacaagtgatatgaccctagaactcaagaatcagcaagaagatattaataataacaaaaagcaagaagaaaggatgttgaaacaaatagaaaatcttcaagaaacagaaacccaattaagaaatgaactagaatatgtgagagaagagctaaaacagaaaagagatgaagttaaatgtaaattggacaagagtgaagaaaattgtaacaatttaaggaaacaagttgaaaataaaaacaagtatattgaagaacttcagcaggagaataaggccttgaaaaaaaaaggtacagcagaaagcaagcaactgaatgtttatgagataaaggtcaataaattagagttagaactagaaagtgccaaacagaaatttggagaaatcacagacacctatcagaaagaaattgaggacaaaaagatatcagaagaaaatcttttggaagaggttgagaaagcaaaagtaatagctgatgaagcagtaaaattacagaaagaaattgataagcgatgtcaacataaaatagctgaaatggtagcacttatggaaaaacataagcaccaatatgataagatcattgaagaaagagactcagaattaggactttataagagcaaagaacaagaacagtcatcactgagagcatctttggagattgaactatccaatctcaaagctgaacttttgtctgttaagaagcaacttgaaatagaaagagaagagaaggaaaaactcaaaagagaggcaaaagaaaacacagctactcttaaagaaaaaaaagacaagaaaacacaaacatttttattggaaacacctgaaatttattggaaattggattctaaagcagttccttcacaaactgtatctcgaaatttcacatcagttgatcatggcatatccaaagataaaagagactatctgtggacatctgccaaaaatactttatctacaccattgccaaaggcatatacagtgaagacaccaacaaaaccaaaactacagcaaagagaaaacttgaatatacccattgaagaaagtaaaaaaaagagaaaaatggcctttgaatttgatattaattcagatagttcagaaactactgatcttttgagcatggtttcagaagaagagacattgaaaacactgtataggaacaataatccaccagcttctcatctttgtgtcaaaacaccaaaaaaggccccttcatctctaacaacccctggatctacactgaagtttggagctataagaaaaatgcgggaggaccgttgggctgtaattgctaaaatggatagaaaaaaaaaactaaaagaagctgaaaagttatttgtttaatttcagagaatcagtgtagttaaggagcctaataacgtgaaacttatagttaatattttgttcttatttgccagagccaaattttatctggaagttgagacttaaaaaatacttgcatgaatgatttgtgtttctttatatttttagcctaaatgttaactacatattgtctggaaacctgtcattgtattcagataattagatgattatatattgttgttactttttcttgtattcatgaaaactgtttttactaagttttcaaatttgtaaagttagcctttgaatgctaagaatgcattattgagggtcattctttattctttactattaaaatattttggatgcaaaaaaaaaaaaaaa

SEQ ID NO: 43 - DNA, 3665 bp, Homo sapiens
ggcttggggcagccgggtagctcggaggtcgtggcgctgggggctagcaccagcgctctgtcgggaggcgcagcggttaggtggaccggtcagcggactcaccggccagggcgctcggtgctggaatttgatattcattgatccgggttttatccctcttcttttttcttaaacatttttttttaaaactgtattgtttctcgttttaatttatttttgcttgccattccccacttgaatcgggccgacggcttggggagattgctctacttccccaaatcactgtggattttggaaaccagcagaaagaggaaagaggtagcaagagctccagagagaagtcgaggaagagagagacggggtcagagagagcgcgcgggcgtgcgagcagcgaaagcgacaggggcaaagtgagtgacctgcttttgggggtgaccgccggagcgcggcgtgagccctcccccttgggatcccgcagctgaccagtcgcgctgacggacagacagacagacaccgcccccagccccagctaccacctcctccccggccggcggcggacagtggacgcggcggcgagccgcgggcaggggccggagcccgcgcccggaggcggggtggagggggtcggggctcgcggcgtcgcactgaaacttttcgtccaacttctgggctgttctcgcttcggaggagccgtggtccgcgcgggggaagccgagccgagcggagccgcgagaagtgctagctcgggccgggaggagccgcagccggaggagggggaggaggaagaagagaaggaagaggagagggggccgcagtggcgactcggcgctcggaagccgggctcatggacgggtgaggcggcggtgtgcgcagacagtgctccagccgcgcgcgctccccaggccctggcccgggcctcgggccggggaggaagagtagctcgccgaggcgccgaggagagcgggccgccccacagcccgagccggagagggagcgcgagccgcgccggccccggtcgggcctccgaaaccatgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaaaaaatcagttcgaggaaagggaaaggggcaaaaacgaaagcgcaagaaatcccggtataagtcctggagcgtgtacgttggtgcccgctgctgtctaatgccctggagcctccctggcccccatccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtgagccgggcaggaggaaggagcctccctcagggtttcgggaaccagatctctcaccaggaaagactgatacagaacgatcgatacagaaaccacgctgccgccaccacaccatcaccatcgacagaacagtccttaatccagaaacctgaaatgaaggaagaggagactctgcgcagagcactttgggtccggagggcgagactccggcggaagcattcccgggcgggtgacccagcacggtccctcttggaattggattcgccattttatttttcttgctgctaaatcaccgagcccggaagattagagagttttatttctgggattcctgtagacacacccacccacatacatacatttatatatatatatattatatatatataaaaataaatatctctattttatatatataaaatatatatattctttttttaaattaacagtgctaatgttattggtgtcttcactggatgtatttgactgctgtggacttgagttgggaggggaatgttcccactcagatcctgacagggaagaggaggagatgagagactctggcatgatcttttttttgtcccacttggtggggccagggtcctctcccctgcccaggaatgtgcaaggccagggcatgggggcaaatatgacccagttttgggaacaccgacaaacccagccctggcgctgagcctctctaccccaggtcagacggacagaaagacagatcacaggtacagggatgaggacaccggctctgaccaggagtttggggagcttcaggacattgctgtgctttggggattccctccacatgctgcacgcgcatctcgcccccaggggcactgcctggaagattcaggagcctgggcggccttcgcttactctcacctgcttctgagttgcccaggagaccactggcagatgtcccggcgaagagaagagacacattgttggaagaagcagcccatgacagctccccttcctgggactcgccctcatcctcttcctgctccccttcctggggtgcagcctaaaaggacctatgtcctcacaccattgaaaccactagttctgtccccccaggagacctggttgtgtgtgtgtgagtggttgaccttcctccatcccctggtccttcccttcccttcccgaggcacagagagacagggcaggatccacgtgcccattgtggaggcagagaaaagagaaagtgttttatatacggtacttatttaatatccctttttaattagaaattaaaacagttaatttaattaaagagtagggttttttttcagtattcttggttaatatttaatttcaactatttatgagatgtatcttttgctctctcttgctctcttatttgtaccggtttttgtatataaaattcatgtttccaatctctctctccctgatcggtgacagtcactagcttatcttgaacagatatttaattttgctaacactcagctctgccctccccgatcccctggctccccagcacacattcctttgaaataaggtttcaatatacatctacatactatatatatatttggcaacttgtatttgtgtgtatatatatatatatatgtttatgtatatatgtgattctgataaaatagacattgctattctgttttttatatgtaaaaacaaaacaagaaaaaatagagaattctacatactaaatctctctccttttttaattttaatatttgttatcatttatttattggtgctactgtttatccgtaataattgtggggaaaagatattaacatcacgtctttgtctctagtgcagtttttcgagatattccgtagtacatatttatttttaaacaacgacaaagaaatacagatatatcttaaaaaaaaaaaagcattttgtattaaagaatttaattctgatctcaaaaaaaaaaaaa

SEQ ID NO: 44 - DNA, 2204 bp, Homo sapiens
gagcggtgcggaggctctgctcggatcgaggtctgcagcgcagcttcgggagcatgagtgctgcagtgactgcagggaagctggcacgggcaccggccgaccctgggaaagccggggtccccggagttgcagctcccggagctccggcggcggctccaccggcgaaagagatcccggaggtcctagtggacccacgcagccggcggcgctatgtgcggggccgctttttgggcaagggcggctttgccaagtgcttcgagatctcggacgcggacaccaaggaggtgttcgcgggcaagattgtgcctaagtctctgctgctcaagccgcaccagagggagaagatgtccatggaaatatccattcaccgcagcctcgcccaccagcacgtcgtaggattccacggctttttcgaggacaacgacttcgtgttcgtggtgttggagctctgccgccggaggtctctcctggagctgcacaagaggaggaaagccctgactgagcctgaggcccgatactacctacggcaaattgtgcttggctgccagtacctgcaccgaaaccgagttattcatcgagacctcaagctgggcaaccttttcctgaatgaagatctggaggtgaaaataggggattttggactggcaaccaaagtcgaatatgacggggagaggaagaagaccctgtgtgggactcctaattacatagctcccgaggtgctgagcaagaaagggcacagtttcgaggtggatgtgtggtccattgggtgtatcatgtataccttgttagtgggcaaaccaccttttgagacttcttgcctaaaagagacctacctccggatcaagaagaatgaatacagtattcccaagcacatcaaccccgtggccgcctccctcatccagaagatgcttcagacagatcccactgcccgcccaaccattaacgagctgcttaatgacgagttctttacttctggctatatccctgcccgtctccccatcacctgcctgaccattccaccaaggttttcgattgctcccagcagcctggaccccagcaaccggaagcccctcacagtcctcaataaaggcttggagaaccccctgcctgagcgtccccgggaaaaagaagaaccagtggttcgagagacaggtgaggtggtcgactgccacctcagtgacatgctgcagcagctgcacagtgtcaatgcctccaagccctcggagcgtgggctggtcaggcaagaggaggctgaggatcctgcctgcatccccatcttctgggtcagcaagtgggtggactattcggacaagtacggccttgggtatcagctctgtgataacagcgtgggggtgctcttcaatgactcaacacgcctcatcctctacaatgatggtgacagcctgcagtacatagagcgtgacggcactgagtcctacctcaccgtgagttcccatcccaactccttgatgaagaagatcaccctccttaaatatttccgcaattacatgagcgagcacttgctgaaggcaggtgccaacatcacgccgcgcgaaggtgatgagctcgcccggctgccctacctacggacctggttccgcacccgcagcgccatcatcctgcacctcagcaacggcagcgtgcagatcaacttcttccaggatcacaccaagctcatcttgtgcccactgatggcagccgtgacctacatcgacgagaagcgggacttccgcacataccgcctgagtctcctggaggagtacggctgctgcaaggagctggccagccggctccgctacgcccgcactatggtggacaagctgctgagctcacgctcggccagcaaccgtctcaaggcctcctaatagctgccctcccctccggactggtgccctcctcactcccacctgcatctggggcccatactggttggctcccgcggtgccatgtctgcagtgtgccccccagccccggtggctgggcagagctgcatcatccttgcaggtgggggttgctgtgtaagttatttttgtacatgttcgggtgtgggttctacagccttgtccccctccccctcaaccccaccatatgaattgtacagaatatttctattgaattcggaactgtcctttccttggctttatgcacattaaacagatgtgaatattcaaaaaaaaaaaaaaaaaaaaaaaaaaaaaaaaaaa

(19) United States
(12) Patent Application Publication
(10) Pub. No.: US 2006/0159689 A1
(43) Pub. Date: Jul. 20, 2006

(54) Combinations of tumor-associated antigens in diagnostics for various types of cancers

(76) Inventors: Chih-Sheng Chiang, Chatsworth, CA (US); John J. L. Simard, West Vancouver (CA); David C. Diamond, West Hills, CA (US); Adrian Ion Bot, Valencia, CA (US); Xiping Liu, Temple City, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/323,964

(22) Filed: Dec. 29, 2005

Related U.S. Application Data
(63) Continuation-in-part of application No. 11/155,288, filed on Jun. 17, 2005.
(60) Provisional application No. 60/580,969, filed on Jun. 17, 2004.

(30) Foreign Application Priority Data
Jun. 17, 2005 (WO) PCT/US05/21836

(51) Int. Cl. G01N 33/574; A61K 39/395

(52) U.S. Cl. 424/155.1; 435/7.23

(57) ABSTRACT

Disclosed herein are methods for matching a cancer condition with an appropriate immunotherapeutic agent and/or regimen. Also disclosed are methods for confirming diagnosis of a particular type of cancer. Embodiments of the invention disclosed herein are directed to the use of effective combinations of TuAAs to optimize the match between a patient's cancer condition and available immunotherapies.

### CROSS REFERENCE TO RELATED APPLICATIONS

This application is a continuation-in-part of U.S. application Ser. No. 11/155,288, and PCT Application No. PCT/US2005/021836, both filed on Jun. 17, 2005, which claim priority under 35 U.S.C. § 119(e) to U.S. Provisional Application No. 60/580,969, filed on Jun. 17, 2004, each entitled COMBINATIONS OF TUMOR-ASSOCIATED ANTIGENS IN DIAGNOSTICS FOR VARIOUS TYPES OF CANCERS; each of which is incorporated herein by reference in its entirety.

### BACKGROUND OF THE INVENTION

1. Field of the Invention

Disclosed herein are methods for matching a cancer condition with an appropriate immunotherapeutic agent and/or regimen. Also disclosed are methods for confirming diagnosis of a particular type of cancer.

2. Description of the Related Art

The American Cancer Society has estimated that over one million people get cancer each year, and that approximately one out of every two American men and one out of every three American women will have some type of cancer at some point during their lifetime.

Cancer generally develops when cells in a part of the body begin to grow out of control. Although there are many kinds of cancer, they typically begin with out-of-control growth of abnormal cells.

Normal body cells grow, divide, and die in an orderly fashion. Cancer cells are different in that they continue to grow and divide. Instead of dying, they outlive normal cells and continue to form new abnormal cells.

Usual treatment options for cancer include surgery, radiation therapy, and chemotherapy. A fourth branch of treatment is developing, which is referred to as immunotherapy. Immunotherapies attempt to help the immune system recognize cancer cells, and/or to strengthen a response against cancer cells in order to destroy the cancer. Immunotherapies include active and passive immunotherapies. Active immunotherapies attempt to stimulate the body's own immune system to fight the disease. Passive immunotherapies generally do not rely on the body to attack the disease; instead, they use immune system components (such as antibodies) created outside of the body.

A continuing need exists for treatment options that are more closely matched to a patient's cancer condition or type. In addition, there is a need for more accurate diagnostic tools for cancer.

### SUMMARY OF THE INVENTION

Embodiments of the invention disclosed herein are directed to the use of a preselected panel of tumor-associated antigens (TuAAs) to match a patient's cancer condition or type with an appropriate immunotherapeutic product or regimen. In some embodiments, the TuAAs are antigens expressed by the cancer cell itself. In some embodiments, the TuAAs are antigens associated with non-cancerous components of the tumor, such as tumor-associated neovasculature or other stroma. In some embodiments, the panels further include a tumor growth factor and/or a signal transduction protein. Methods to determine, diagnose, or confirm a diagnosis of a type of cancer using a preselected panel of antigens are also disclosed. Methods for predicting disease progression in a cancer patient are also disclosed.

Some embodiments of the invention are directed to methods for matching a patient's cancer condition or type with an immunotherapeutic product and/or regimen including the steps of assaying the patient's tumor tissue for expression of antigens in a preselected panel, and based on the assay results, selecting an immunotherapeutic agent targeting one, or two, or three or more of the antigens expressed by the patient's tumor tissue. The methods can further include the step of developing an antigen profile for the tumor and selecting the immunotherapeutic product and/or regimen based on the profile. In some embodiments, the selected product is an active immunotherapeutic. In some embodiments, the product comprises an immunogen that includes or encodes at least a portion of at least one of the expressed antigens. In some embodiments, the selected product is a passive immunotherapeutic. In some embodiments, the product comprises a monoclonal antibody.

Some embodiments relate to methods for preparing a cancer immunotherapeutic product, wherein the immunotherapeutic product is selected on the basis of the expression profile of tumor tissue for at least two tumor-associated antigens (TuAAs) in a preselected panel so that the immunotherapeutic product comprises or encodes at least a segment of at least one of the expressed TuAAs, and wherein the immunotherapeutic product is optionally combined with pharmaceutically acceptable excipients.

Embodiments of the invention are also directed to methods of matching a patient's cancer condition with an immunotherapeutic regimen including the steps of assaying the patient's tumor tissue for two or more expressed tumor associated antigens (TuAAs) in a preselected panel of antigens to develop an antigen profile for the tumor, and selecting an immunotherapeutic regimen based on the antigen profile. In some embodiments, the regimen comprises administering at least one immunotherapeutic product targeting two, three, four, or more of the expressed antigens. The immunotherapeutic products can be in forms such as, for example, nucleic acids, or polypeptides, or cellular, or humoral, or active, or passive, etc. For embodiments in which the regimen comprises administering two or more immunotherapeutic products, the products can be similar in form or different in form. Thus, in some embodiments, the regimen can include both an active immunotherapeutic product and a passive immunotherapeutic product.

In some embodiments, methods for matching a cancer condition in a patient with an immunotherapeutic product are disclosed. The methods can include the steps of: determining the patient's class I MHC type; assaying the patient's tumor tissue for two or more expressed tumor-associated antigens (TuAAs) in a preselected panel; assaying the patient's tumor tissue for the expression of MHC class I or β2-microglobulin; and selecting an immunotherapeutic product for administration to the patient based on the assays, wherein the immunotherapeutic product comprises or encodes an epitope restricted by the patient's class I MHC type, for each of two or more antigens expressed by the tumor. In some embodiments, antigen expression is detected on neoplastic cells, or tumor-associated stromal cells, or both. In some embodiments, the two or more antigens expressed by the tumor include an antigen expressed by a neoplastic cell and an antigen expressed by a tumor-associated stromal cell.

Other embodiments are directed to determining, establishing, or confirming the diagnosis of a type of cancer including the steps of assaying a patient's tumor tissue to detect one or more expressed polypeptides in a preselected panel, wherein the panel comprises two, or three, or four or more TuAAs and at least one lineage specific marker; and confirming the cancer diagnosis based on the assay. In one embodiment, the panel comprises at least two, or three, or four or more TuAAs selected from the group consisting of NY-ESO-1, CEA, PSA, PSCA, SCP-1, PSMA, PRAME, tyrosinase, melan-A/MART-1, an SSX protein, such as SSX-2 or SSX-4, a MAGE protein, such as MAGE-1 or MAGE-3, LAGE, mesothelin, Her-2/Neu, PLK1, VEGF-A, VEGFR2, Tie-2, and the like, or subsets thereof. In some embodiments, the lineage specific marker is a TuAA; in some embodiments the lineage specific marker is not a TuAA. For melanoma, the lineage specific marker can be, for example, tyrosinase, melan-A/MART-1, or gp100. For breast cancer, the lineage specific marker can be, for example, mammaglobin or prolactin-inducuble protein (Brst2). For colon cancer, the lineage specific marker can be, for example, CEA. For lung cancer, the lineage specific antigen can be, for example, thyroid transcription factor 1 (TTF1). For prostate cancer, the lineage specific marker can be, for example, PSA or PSMA.

Some embodiments relate to methods of determining or confirming the occurrence of cancer comprising the step of determining the expression profile of tumor tissue for at least one polypeptide, wherein the polypeptide is part of a preselected panel comprising at least two TuAAs and at least one lineage marker.

The preselected panel of TuAAs can include, for example, but is not limited to, cancer testis antigens, tissue specific antigens, oncofetal antigens, differentiation antigens, growth factors, growth factor receptors, adhesion factors, signal transduction proteins, transcription factors, oncogene products, tumor suppressor gene products, microbial agents, and the like. In some embodiments, the preselected panel comprises two, or three, or more antigens selected from the group consisting of NY-ESO-1, CEA, PSA, PSCA, SCP-1, PSMA, PRAME, tyrosinase, melan-A/MART-1, an SSX protein, such as SSX-2 or SSX-4, a MAGE protein, such as MAGE-1 or MAGE-3, LAGE, mesothelin, Her-2/Neu, PLK1, VEGF-A, VEGFR2, Tie-2, and the like, or subsets thereof. In some embodiments, the panel includes a growth factor, such as VEGF-A. In some embodiments, the panel includes a transduction protein, such as PLK1. In some embodiments, the cancer is carcinoma. The carcinoma can be, for example, breast, colorectal, prostate, pancreatic, lung, ovarian, renal cell, esophageal, head and neck, or melanocyte.

The tumor tissue assayed can include primary tumor tissue or metastatic tumor tissue. Antigen expression can be detected on neoplastic cells, or tumor-associated stromal cells, or both. In some embodiments, the preselected panel includes an antigen expressed by a neoplastic cell and an antigen expressed by a tumor-associated stromal cell. The stromal cells can be neovasculature. The neovasculature associated antigen can be PSMA and the neoplastic cell antigen can be NY-ESO-1, SSX-2, LAGE, or PRAME.

Antigen expression can be detected, directly or indirectly. For example, the assay can detect the absence, presence and/or abundance of mRNA, polypeptide, mature protein, peptide, or MHC-peptide complex. In some embodiments, the assay detects the condition of the TuAAs, such as processing state, differential splicing, mutation from germline, variation from consensus sequence in human population, cellular localization, subcellular localization, co-expression with other markers, and the like. Examples of useful assays include RT-PCR, transcript determination, protein determination, epitope determination, or any combination thereof. In some embodiments, the assay comprises reverse transcription polymerase chain reaction (RT-PCR), real-time PCR, quantitative PCR, northern hybridization, autoradiography, chemiluminescent detection, autofluorography, flow cytometry, gene chip expression profiling, immunohistochemistry, western hybridization, radioimmunoassay, or in situ hybridization, individually or in any combination thereof. In some embodiments, at least two assaying steps are carried out at different time points during the course of disease and comparative information is obtained from the assaying steps. The obtained information can be used to implement, modify or withdraw a therapy.

In one embodiment, the tumor is melanoma and the preselected panel of antigens comprises at least two, or three, or four or more TuAAs selected from the group consisting of tyrosinase, melan-A/MART-1, NY-ESO-1, PRAME, an SSX protein, and a MAGE protein. The SSX protein can be SSX-2 or SSX-4. The MAGE protein can be MAGE-1 or MAGE-3.

In another embodiment, the tumor is breast cancer and the preselected panel of antigens comprises at least two, or three, or four or more TuAAs selected from the group consisting of NY-ESO-1, C35, Her2/Neu, an SSX protein, and a MAGE protein. The SSX protein can be SSX-2 or SSX-4. The MAGE protein can be MAGE-1 or MAGE-3.

In yet another embodiment, the tumor is colorectal cancer and the preselected panel of antigens comprises at least two, or three, or four or more TuAAs selected from the group consisting of CEA, an SSX protein, PRAME, NY-ESO-1, LAGE, PSCA, SCP-1, PSMA, and a MAGE protein. The SSX protein can be SSX-2 or SSX-4. The MAGE protein can be MAGE-1 or MAGE-3.

In yet a further embodiment, the tumor is ovarian cancer and the preselected panel of antigens comprises at least two, or three or four or more TuAAs selected from the group consisting of an SSX protein, PRAME, NY-ESO-1, PSMA, Her2/neu, C35, PSCA, SCP-1, CEA, LAGE, and a MAGE protein. The SSX protein can be SSX-2 or SSX-4. The MAGE protein can be MAGE-1 or MAGE-3. The ovarian cancer can be, for example, serous carcinoma, non-serous carcinoma, mucinous (cell) carcinoma, clear cell carcinoma, and the like.

In still another embodiment, the tumor is lung cancer and the preselected panel of antigens comprises at least two, or three, or four or more TuAAs selected from the group consisting of PSMA, NY-ESO-1, SSX-2, and a MAGE protein. The cancer can be, for example, non-small cell lung cancer. The MAGE protein can be MAGE-1 or MAGE-3.

In a further embodiment, the tumor is prostate cancer and the preselected panel of antigens comprises at least two, or three, or four or more TuAAs selected from the group consisting of NY-ESO-1, PSA, PSCA, PSMA, an SSX protein, and a MAGE protein. The SSX protein can be SSX-2 or SSX-4. The MAGE protein can be MAGE-1 or MAGE-3.

In another embodiment, the tumor is pancreatic cancer and the panel of antigens comprises at least two, or three, or four or more TuAAs selected from the group consisting of PSMA, PRAME, NY-ESO, LAGE, PSCA, and a MAGE protein and an SSX protein. The SSX protein can be SSX-2 or SSX-4. The MAGE protein can be MAGE-1 or MAGE-3.

In still another embodiment, the tumor is renal cell carcinoma and the panel of antigens comprises at least two, or three, or four or more TuAAs selected from the group consisting of PSMA, PRAME, NY-ESO, LAGE, PSCA, SCP-1, a MAGE protein, and an SSX protein. The SSX protein can be SSX-2 or SSX-4. The MAGE protein can be MAGE-1 or MAGE-3.

Another embodiment relates to a method of marketing cancer immunotherapeutics comprising establishing a relationship with a cancer diagnostics laboratory, wherein the laboratory includes TuAA expression in it standard panel of tests, and wherein the TuAAs assayed for correspond to the immunogens of the immunotherapeutics to be marketed, and sending a report with each patient's test results identifying immunotherapeutics comprising immunogens that correspond to the TuAAs expressed by the patient's tumor. In some embodiments, the relationship comprises contract services. In some embodiments, the relationship is a partnership, ownership, joint venture, and the like.

In some embodiments, medicaments comprising an immunotherapeutic agent correlated with a disease profile, wherein the disease profile indicates presence of at least three disease characteristics, comprising at least two expressed tumor-associated antigens (TuAAs), and at least one of a growth factor or a signal transduction protein, and wherein the immunotherapeutic agent targets one or more of the expressed antigens in the profile are disclosed. In some embodiments, at least one of the TuAAs is selected from the group consisting of a cancer testis antigen, a tissue-specific antigen, an oncofetal antigen, a differentiation antigen, a growth factor, a growth factor receptor, an adhesion factor, a signal transduction protein, a transcription factor, an oncogene product, a tumor suppressor gene product, and a microbial antigen. In some embodiments, the profile indicates presence of two or more antigens selected from the group consisting of an SSX protein, SSX-2, SSX-4, a MAGE protein, MAGE-1, MAGE-3, PRAME, NY-ESO-1, LAGE, PSMA, PSCA, SCP-1, melan-A/MART-1 and tyrosinase; and the disease of the disease profile is carcinoma. In some embodiments, the carcinoma is selected from the group consisting of breast, colorectal, prostate, pancreatic, lung, ovarian, renal cell, esophageal, head and neck, and melanocyte. In some embodiments, the immunotherapeutic product is an active immunotherapuetic, and in some embodiments, comprises or encodes at least a segment of at least one of the expressed TuAAs. In some embodiments, the immunotherapeutic product is a passive immunotherapeutic, and in some embodiments is a monoclonal antibody.

In some embodiments, the tumor is melanoma and the profile indicates presence of two or more TuAAs selected from the group consisting of tyrosinase, melan-A/MART-1, NY-ESO-1, PRAME, an SSX protein, and a MAGE protein. The SSX protein can be SSX-2 or SSX-4 and the MAGE protein can be MAGE-1 or MAGE-3. In some embodiments, the tumor is breast cancer and the profile indicates presence of two or more TuAAs selected from the group consisting of NY-ESO-1, Her2/neu, an SSX protein, and a MAGE protein. In some embodiments, the the tumor is colorectal cancer and the profile indicates presence of two or more TuAAs selected from the group consisting of CEA, an SSX protein, PRAME, NY-ESO, LAGE, PSCA, SCP-1, PSMA and a MAGE protein. In some embodiments, the tumor is lung cancer and the profile indicates presence of two or more TuAAs selected from the group consisting of PSMA, NY-ESO-1, SSX-2, and a MAGE protein. In some embodiments, the tumor is prostate cancer and the profile indicates presence of two or more TuAAs selected from the group consisting of NY-ESO-1, PSA, PSCA, PSMA, an SSX protein, and a MAGE protein. In some embodiments, the tumor is ovarian cancer and the profile indicates presence of two or more TuAAs selected from the group consisting of PRAME, PSMA, PSCA, a MAGE protein, SCP-1, an SSX protein, CEA, Her-2/Neu, NY-ESO-1, and LAGE. The ovarian cancer can be serous carcinoma, non-serous carcinoma, mucinous (cell) carcinoma, or clear cell carcinoma. In some embodiments, the tumor is renal cancer and the profile indicates presence of two or more TuAAs selected from the group consisting of an SSX protein, PRAME, NY-ESO, LAGE, PSCA, SCP-1, PSMA and a MAGE protein. In some embodiments, the tumor is pancreatic cancer and the profile indicates presence of two or more TuAAs selected from the group consisting of an SSX protein, PRAME, NY-ESO, LAGE, PSCA, PSMA, and a MAGE protein.

### BRIEF DESCRIPTION OF THE DRAWINGS

FIG. 1 is a timeline depicting the schedule of immunization with two plasmid (pCBP expressing SSX-2 41-49 and pSEM expressing Melan A).

FIG. 2 is a bar graph that shows CTL activity obtained using the protocol in FIG. 1.

FIG. 3 is a timeline depicting the schedule of immunization of an entrain-and-amplify immunization protocol using plasmids and peptides representing two epitopes.

FIG. 4 is a table showing in vivo clearance of epitope-pulsed cells in mice immunized according to the protocol of FIG. 3.

FIGS. 5A and 5B are timelines depicting immunization protocols for inducing strong multivalent responses. FIG. 5A shows the use of peptides for boosting restores multivalent immune responses even if plasmids and peptides are used as mixtures. FIG. 5B shows segregation of plasmid and peptide components allows induction of multivalent immune responses.

### DETAILED DESCRIPTION OF THE PREFERRED EMBODIMENT

The frequency of expression of many tumor-associated antigens (TuAAs) in various types of cancers is known. However, the frequency of appearance of some antigens, and especially certain combinations of TuAAs, in various types of cancers has not been reported. Accurate measurement of the presence of TuAAs in tumor tissues aids in determining which TuAAs will be useful for the treatment of a particular type of cancer.

Many attempts to develop immunotherapies for cancer have targeted a single antigen. This can be problematic for two distinct reasons. Firstly, the expression of any particular TuAA in cancer can be mosaic with the antigen expression ranging from high in some cells within a tumor mass to completely absent in others. Moreover, the TuAA may be expressed in some lesions but not others. By directing an immune response against more than a single antigen, if properly selected, the number of tumor cells that can be recognized is maximized. Secondly, some tumors lose expression of a TuAA following immunization, giving rise to a resistant population. If the immune response is directed against more than one TuAA it becomes much more difficult for a resistant tumor to arise because it must then simultaneously lose expression of each of the antigens in order to escape. Thus, in treating cancer with immunotherapy, it can be advantageous to use a combination of TuAAs both due to more complete coverage of the population of tumor cells, and because there will be less chance of tumor escape through loss of expression of the TuAAs. In preferred embodiments, this multivalent attack technique is employed when a tumor is positive for two, three, four or more TuAAs of the combination used.

Multivalent attack can offer another advantage in increasing the sensitivity of the tumor to attack. If more than a single antigen on a tumor cell is targeted, the effective concentration of anti-tumor agent is increased. In addition, attack on stroma associated with the tumor, such as vasculature, can increase the accessibility of the tumor cells to the agent(s) targeting them. Thus, even an antigen that is also expressed on some normal tissue can receive greater consideration as a target antigen, if the other antigens to be targeted in a multivalent attack are not also expressed by that tissue.

In some embodiments, practice of the methods includes use of at least two different compositions and, especially when there is more than a single target antigen, can involve several compositions to be administered together and/or at different times. Thus, embodiments of the invention include sets and subsets of immunogenic compositions and individual doses thereof. Multivalency can be achieved using compositions comprising multivalent immunogens, combinations of monovalent immunogens, coordinated use of compositions comprising one or more monovalent immunogens or various combinations thereof. Multiple compositions, manufactured for use in a particular treatment regimen or protocol according to such methods, define an immunotherapeutic product. In some embodiments all or a subset of the compositions of the product are packaged together in a kit.

Definitions

Unless otherwise clear from the context of the use of a term herein, the following listed terms shall generally have the indicated meanings for purposes of this description.

PROFESSIONAL ANTIGEN-PRESENTING CELL (pAPC)—a cell that possesses T cell costimulatory molecules and is able to induce a T cell response. Well characterized pAPCs include dendritic cells, B cells, and macrophages.

PERIPHERAL CELL—a cell that is not a pAPC.

HOUSEKEEPING PROTEASOME—a proteasome normally active in peripheral cells, and generally not present or not strongly active in pAPCs.

IMMUNOPROTEASOME—a proteasome normally active in pAPCs; the immunoproteasome is also active in some peripheral cells in infected tissues or following exposure to interferon.

EPITOPE—a molecule or substance capable of stimulating an immune response. In preferred embodiments, epitopes according to this definition include but are not necessarily limited to a polypeptide and a nucleic acid encoding a polypeptide, wherein the polypeptide is capable of stimulating an immune response. In other preferred embodiments, epitopes according to this definition include but are not necessarily limited to peptides presented on the surface of cells, the peptides being non-covalently bound to the binding cleft of class I MHC, such that they can interact with T cell receptors (TCR). Epitopes presented by class I MHC may be in immature or mature form. “Mature” refers to an MHC epitope in distinction to any precursor (“immature”) that may include or consist essentially of a housekeeping epitope, but also includes other sequences in a primary translation product that are removed by processing, including without limitation, alone or in any combination, proteasomal digestion, N-terminal trimming, or the action of exogenous enzymatic activities. Thus, a mature epitope may be provided embedded in a somewhat longer polypeptide, the immunological potential of which is due, at least in part, to the embedded epitope; likewise, the mature epitope can be provided in its ultimate form that can bind in the MHC binding cleft to be recognized by TCR.

MHC EPITOPE—a polypeptide having a known or predicted binding affinity for a mammalian class I or class II major histocompatibility complex (MHC) molecule.

HOUSEKEEPING EPITOPE—In a preferred embodiment, a housekeeping epitope is defined as a polypeptide fragment that is an MHC epitope, and that is displayed on a cell in which housekeeping proteasomes are predominantly active. In another preferred embodiment, a housekeeping epitope is defined as a polypeptide containing a housekeeping epitope according to the foregoing definition, that is flanked by one to several additional amino acids. In another preferred embodiment, a housekeeping epitope is defined as a nucleic acid that encodes a housekeeping epitope according to the foregoing definitions. Exemplary housekeeping epitopes are provided in U.S. patent application Ser. No. 10/117,937, filed on Apr. 4, 2002 (Pub. No. 2003-0220239 A1), Ser. No. 11/067,159 (Pub. No. 2005-0221440 A1), filed Feb. 25, 2005, Ser. No. 11/067,064 (Pub. No. 2005-0142144 A1), filed Feb. 25, 2005, and Ser. No. 10/657,022 (Pub. No. 2004-0180354 A1), filed Sep. 5, 2003, and in PCT Application No. PCT/US2003/027706 (Pub. No. WO 04/022709 A2), filed Sep. 5, 2003; and U.S. Provisional Patent Application Nos. 60/282,211, filed on Apr. 6, 2001; 60/337,017, filed on Nov. 7, 2001; 60/363,210 filed Mar. 7, 2002; and 60/409,123, filed on Sep. 6, 2002. Each of the listed applications is entitled “EPITOPE SEQUENCES.” Each of the applications mentioned in this paragraph is incorporated herein by reference in its entirety.

IMMUNE EPITOPE—In a preferred embodiment, an immune epitope is defined as a polypeptide fragment that is an MHC epitope, and that is displayed on a cell in which immunoproteasomes are predominantly active. In another preferred embodiment, an immune epitope is defined as a polypeptide containing an immune epitope according to the foregoing definition, that is flanked by one to several additional amino acids. In another preferred embodiment, an immune epitope is defined as a polypeptide including an epitope cluster sequence, having at least two polypeptide sequences having a known or predicted affinity for a class I MHC. In yet another preferred embodiment, an immune epitope is defined as a nucleic acid that encodes an immune epitope according to any of the foregoing definitions.

TARGET CELL—In a preferred embodiment, a target cell is a cell associated with a pathogenic condition that can be acted upon by the components of the immune system, for example, a cell infected with a virus or other intracellular parasite, or a neoplastic cell. In another embodiment, a target cell is a cell to be targeted by the vaccines and methods of the invention. Examples of target cells according to this definition include but are not necessarily limited to: a neoplastic cell and a cell harboring an intracellular parasite, such as, for example, a virus, a bacterium, or a protozoan. Target cells can also include cells that are targeted by CTL as a part of an assay to determine or confirm proper epitope liberation and processing by a cell expressing immunoproteasome, to determine T cell specificity or immunogenicity for a desired epitope. Such cells can be transformed to express the liberation sequence, or the cells can simply be pulsed with peptide/epitope.

TARGET-ASSOCIATED ANTIGEN (TAA)—a protein or polypeptide present in a target cell.

TUMOR-ASSOCIATED ANTIGEN (TuAA)—a TAA, wherein the target cell is a neoplastic cell. In alternate embodiments, a TuAA is an antigen associated with non-cancerous cells of the tumor such as tumor neovasculature or other stromal cells within the tumor microenvironment.

HLA EPITOPE—a polypeptide having a known or predicted binding affinity for a human class I or class II HLA complex molecule.

ANTIBODY—a natural immunoglobulin (Ig), poly- or monoclonal, or any molecule composed in whole or in part of an Ig binding domain, whether derived biochemically, or by use of recombinant DNA, or by any other means. Examples include inter alia, F(ab), single chain Fv, and Ig variable region-phage coat protein fusions.

SUBSTANTIAL SIMILARITY—this term is used to refer to sequences that differ from a reference sequence in an inconsequential way as judged by examination of the sequence. Nucleic acid sequences encoding the same amino acid sequence are substantially similar despite differences in degenerate positions or minor differences in length or composition of any non-coding regions. Amino acid sequences differing only by conservative substitution or minor length variations are substantially similar. Additionally, amino acid sequences comprising housekeeping epitopes that differ in the number of N-terminal flanking residues, or immune epitopes and epitope clusters that differ in the number of flanking residues at either terminus, are substantially similar. Nucleic acids that encode substantially similar amino acid sequences are themselves also substantially similar.

FUNCTIONAL SIMILARITY—this term is used to refer to sequences that differ from a reference sequence in an inconsequential way as judged by examination of a biological or biochemical property, although the sequences may not be substantially similar. For example, two nucleic acids can be useful as hybridization probes for the same sequence but encode differing amino acid sequences. Two peptides that induce cross-reactive CTL responses are functionally similar even if they differ by non-conservative amino acid substitutions (and thus may not be within the substantial similarity definition). Pairs of antibodies, or TCRs, that recognize the same epitope can be functionally similar to each other despite whatever structural differences exist. Testing for functional similarity of immunogenicity can be conducted by immunizing with the “altered” antigen and testing the ability of an elicited response, including but not limited to an antibody response, a CTL response, cytokine production, and the like, to recognize the target antigen. Accordingly, two sequences may be designed to differ in certain respects while retaining the same function. Such designed sequence variants of disclosed or claimed sequences are among the embodiments of the present invention.

EXPRESSION CASSETTE—a polynucleotide sequence encoding a polypeptide, operably linked to a promoter and other transcription and translation control elements, including but not limited to enhancers, termination codons, internal ribosome entry sites, and polyadenylation sites. The cassette can also include sequences that facilitate moving it from one host molecule to another.

EMBEDDED EPITOPE—in some embodiments, an embedded epitope is an epitope that is wholly contained within a longer polypeptide; in other embodiments, the term also can include an epitope in which only the N-terminus or the C-terminus is embedded such that the epitope is not wholly in an interior position with respect to the longer polypeptide.

MATURE EPITOPE—a peptide with no additional sequence beyond that present when the epitope is bound in the MHC peptide-binding cleft.

EPITOPE CLUSTER—a polypeptide, or a nucleic acid sequence encoding it, that is a segment of a protein sequence, including a native protein sequence, comprising two or more known or predicted epitopes with binding affinity for a shared MHC restriction element. In preferred embodiments, the density of epitopes within the cluster is greater than the density of all known or predicted epitopes with binding affinity for the shared MHC restriction element within the complete protein sequence. Epitope clusters are disclosed and more fully defined in U.S. patent application Ser. No. 09/561,571, filed Apr. 28, 2000, entitled “EPITOPE CLUSTERS,” which is incorporated herein by reference in its entirety.

LIBERATION SEQUENCE—a designed or engineered sequence comprising or encoding a housekeeping epitope embedded in a larger sequence that provides a context allowing the housekeeping epitope to be liberated by processing activities including, for example, immunoproteasome activity, N terminal trimming, and/or other processes or activities, alone or in any combination.

CTLp—CTL precursors are T cells that can be induced to exhibit cytolytic activity. Secondary in vitro lytic activity, by which CTLp are generally observed, can arise from any combination of naïve, effector, and memory CTL in vivo.

MEMORY T CELL—A T cell, regardless of its location in the body, that has been previously activated by antigen, but is in a quiescent physiologic state requiring re-exposure to antigen in order to gain effector function. Phenotypically they are generally CD62L⁻ CD44^{hi} CD107α⁻ IGN-γ⁻ LTβ⁻ TNF-α⁻ and is in G0 of the cell cycle.

EFFECTOR T CELL—A T cell that, upon encountering antigen, readily exhibits effector function. Effector T cells are generally capable of exiting the lymphatic system and entering the immunological periphery. Phenotypically they are generally CD62L⁻ CD44^{hi} CD107α⁺ IGN-γ⁺ LTβ⁺ TNF-α⁺ and actively cycling.

EFFECTOR FUNCTION—Generally, T cell activation generally, including acquisition of cytolytic activity and/or cytokine secretion.

INDUCING a T cell response—Includes in many embodiments the process of generating a T cell response from naïve, or in some contexts, quiescent cells; activating T cells.

AMPLIFYING a T cell response—Includes in many embodiments, the process or increasing the number of cells, the number of activated cells, the level of activity, rate of proliferation, or similar parameter of T cells involved in a specific response.

ENTRAINMENT—Includes in many embodiments an induction that confers particular stability on the immune profile of the induced lineage of T cells. In various embodiments, the term “entrain” can correspond to “induce,” and/or “initiate.”

TOLL-LIKE RECEPTOR (TLR)— Toll-like receptors (TLRs) are a family of pattern recognition receptors that are activated by specific components of microbes and certain host molecules. As part of the innate immune system, they contribute to the first line of defense against many pathogens, but also play a role in adaptive immunity.

TOLL-LIKE RECEPTOR (TLR) LIGAND—Any molecule capable of binding and activating a toll-like receptor. Examples include, without limitation: poly IC A synthetic, double-stranded RNA known for inducing interferon. The polymer is made of one strand each of polyinosinic acid and polycytidylic acid, double-stranded RNA, unmethylated CpG oligodeoxyribonucleotide or other immunostimulatory sequences (ISSs), lipopolysacharide (LPS), β-glucans, and imidazoquinolines, as well as derivatives and analogues thereof.

IMMUNOPOTENTIATING ADJUVANTS—Adjuvants that activate pAPC or T cells including, for example: TLR ligands, endocytic-Pattern Recognition Receptor (PRR) ligands, quillaja saponins, tucaresol, cytokines, and the like. Some preferred adjuvants are disclosed in Marciani, D. J. Drug Discovery Today 8:934-943, 2003, which is incorporated herein by reference in its entirety.

IMMUNOSTIMULATORY SEQUENCE (ISS)—Generally an oligodeoxyribonucleotide containing an unmethlylated CpG sequence. The CpG may also be embedded in bacterially produced DNA, particularly plasmids. Further embodiments include various analogues; among preferred embodiments are molecules with one or more phosphorothioate bonds or non-physiologic bases.

VACCINE—In preferred embodiments a vaccine can be an immunogenic composition providing or aiding in prevention of disease. In other embodiments, a vaccine is a composition that can provide or aid in a cure of a disease. In others, a vaccine composition can provide or aid in amelioration of a disease. Further embodiments of a vaccine immunogenic composition can be used as therapeutic and/or prophylactic agents.

IMMUNIZATION—a process to induce partial or complete protection against a disease. Alternatively, a process to induce or amplify an immune system response to an antigen. In the second definition it can connote a protective immune response, particularly proinflammatory or active immunity, but can also include a regulatory response. Thus in some embodiments immunization is distinguished from tolerization (a process by which the immune system avoids producing proinflammatory or active immunity) while in other embodiments this term includes tolerization.

ENCODE—an open-ended term such that a nucleic acid encoding a particular amino acid sequence can consist of codons specifying that (poly)peptide, but can also comprise additional sequences either translatable, or for the control of transcription, translation, or replication, or to facilitate manipulation of some host nucleic acid construct.

COVERAGE—the fraction or proportion of tumor cells expressing a particular TuAA or at least one TuAA from a set of selected TuAAs.

REDUNDANCY—the degree to which a population of tumor cells, or some subset of them, express more than one of a selected set of TuAAs.

CO-TARGETING—in preferred embodiments, co-targeting involves inducing and/or amplifying an immune response against a target cell, while also inducing an immune response against at least one other agent in the vicinity and/or milieu of a tumor. In some embodiments, agents within the vicinity and/or milieu of the tumor include, but are not limited to, cancer cells, stromal cells, including those associated with neovasculature, endothelial cells, fibroblasts, inflammatory cells, epithelial cells, autocrine factors, and paracrine factors. In some embodiments, neoplastic cells and stromal cells are specifically targeted. In other embodiments, an immune response is induced and/or amplified against neovasculature and other non-transformed, non-lymphoid cells within the tumor microenvironment. In still other embodiments, an immune response is induced against cancer cells and autocrine and/or paracrine factors produced by cells in the tumor microenvironment.

Cancer Immunotherapy and Diagnosis

Cancer immunotherapy has been strongly influenced by work on melanoma and prostate cancer, as they have been among the earliest and most widely approached targets in the field. Many of the antigens used in the various attempts to develop therapeutic vaccines for these cancers have been differentiation markers, that is antigens specific to that cell type. As a result, the existing paradigm is to develop immunotherapeutics for a particular type of cancer. Patients are then treated with the agent simply because they have been diagnosed with a particular type of cancer. In some instances, a patient's tumor is first evaluated for expression of a particular target antigen. However, many of the TuAAs now known, even some initially classified as differentiation markers, are expressed in many types of cancer. As such it can be useful to classify tumors by the TuAAs that they express rather than, or in addition to, their tissue of origin. Thus, in some embodiments, a single immunotherapeutic, preferably multivalent, can be used to treat a wide variety of tumor types. This is not to say that any particular antigen or combination of antigens will be uniformly useful across all, or even many, tumor types. To the contrary, expression frequency can vary considerably from type to type. Moreover, among the widely expressed TuAAs it is common that they are expressed in only a fraction of any particular tumor type. Indeed, this is part of the impetus to apply immunotherapeutics based on these antigens to various tumor types. Nonetheless, both individual antigens, and combinations of them, will have definable frequencies associated with particular tumor types. Thus, immunotherapeutics designed according to the more favorable frequencies observed can be more effective and/or efficient when applied to those particular tumor types.

Despite the prevalence of certain TuAAs, or combinations of them, in particular tumor types, it is not always sufficient to simply treat patients having a particular type of tumor with an immunotherapeutic targeting a prevalent antigen or antigens expressed by that tumor type. Preferably, patients' tumor tissue should be screened for the expression of TuAAs for which there is a corresponding immunotherapeutic available, whether marketed or in clinical trials. As the number and variety of cancer immunotherapies grow it will be increasingly advantageous to screen any particular patient's tumor tissue for expression of a variety of TuAAs that can be expressed by that tumor type so as to afford the clinician the widest choice in matching a cancer condition with available immunotherapies.

Although much of this disclosure focuses on immunotherapeutic products and/or regimens that actively induce immunity mediated by class I MHC restricted T cells, the matching procedures described are equally applicable with immunotherapies of all kinds, active or passive, cellular or humoral, or any combination thereof. The methods claimed herein are adapted to this developing environment in which there is a substantial number of immunotherapies targeting various antigens. The methods embodied herein optimize the matching between a particular patient's cancer type or condition to available immunotherapeutics. This is in contrast to existing practice that is designed to simply qualify a patient as eligible (or ineligible) for treatment with one particular agent.

Preferably, a panel of TuAAs that are expressed with relatively high frequency in a particular tumor type is assembled and assays established. Accordingly, one embodiment of the invention described herein includes assembly of the panel and establishment of appropriate assays. It can be advantageous to include a TuAA that is widely expressed in a variety of tumor types in the panel.

The methods disclosed herein can begin with an assay of a tumor tissue of the corresponding presumptive type for expression of a preselected panel of antigens. In some embodiments, a panel of TuAAs assembled for one tumor type can be used to screen other tumor types that can express at least some of the same antigens. In some embodiments, an expression profile is developed using the assay results. Selection of an appropriate immunotherapeutic can be based on how well the composition of the immunotherapeutic, such as immunogens (or effector agents in the case of passive immunotherapy), corresponds to the detected antigens of the panel. The panel can include more antigens than are likely to be targeted by any immunotherapeutic of well-defined composition or detected in any one tissue sample.

Thus, it is not necessary that an immunotherapeutic product comprise immunogens corresponding to every antigen in the panel. Nor is it required that the panel antigens all be the target of some set of immunotherapeutic products that could reasonably be combined in a regimen of immunotherapy. Also, although advantageous, it is not required that there is a perfect match between the composition of the immunotherapeutic(s) and the expression profile of the tumor. Heterogeneity of antigen expression by a patient's tumor is common. Thus, there is a significant possibility of an antigen, undetectable in a tissue sample, nonetheless being expressed at another site. This is especially true for the antigens most commonly expressed by the particular tumor type. Thus, a multivalent immunotherapeutic in which one, some, or all of the constituent immunogens correspond to TuAAs expressed by an assayed portion of a patient's tumor can be used to treat that patient according to the judgment of the clinician. Similarly, the patient can be treated with a combination of multi- and monovalent agents to optimize the match between the expression profile and the antigens targeted. Passive immunotherapeutics, in particular, are often monovalent, but the skilled clinician will nonetheless understand how they can be combined with additional passive or active immunotherapeutics into a useful immunotherapeutic regimen. Passive immunotherapies currently known in the art include: trastuzumab (HERCEPTIN®), which targets the TuAA HER2/Neu; bevacizumab (AVASTIN®), which targets VEGF (vascular endothelilal growth factor) to inhibit vascularization of tumors; cetuximab (ERBITUX™), which targets the antigen epidermal growth factor receptor (EGFR, HER1, c-erbB-1); and panitumumab, which also targets the antigen epidermal growth factor receptor. Additionally, there are several mono- and multivalent active immunotherapeutic in development. These include APC8015 (PROVENGE®), which targets prostatic acid phosphatase; APC8024 which targets HER2/Neu; MKC1106 which targets PRAME, PSMA, SSX-2, and NY-ESO-1; pSEM (SYNCHROVAX™ SEM), which targets Melan-A; MKC1207 which targets Melan-A and tyrosinase; pTA2M (SYNCHROTOPE® TA2M), which targets tyrosinase; DCVAX®-prostate, which targets PSMA; ALVAC(2)-gp100M, which targets gp100; ALVAC MAGE 1,3, which targets MAGE-1 and MAGE-3; ALVAC CEA, which target CEA; the NY-ESO-1/ISCOMATRIX™ vaccine, which targets NY-ESO-1; PANVAC™-VF, which targets CEA and MUC-1; and PROSTVAC®-VF, which targets PSA.

Diagnosis of cancer type can be challenging, leading to uncertainty. Similar screening assays can be used to establish or confirm the diagnosis of tumor type by including a lineage specific marker in the panel. The marker can itself be a TuAA, as in tyrosinase for melanoma and PSA for prostate, for example. Alternatively, the marker can be any antigen that is reasonably specific to the cell type in question and the expression of which is maintained in neoplastic cells—for example, mammaglobin for breast tissue.

Assay Technology

Many technologies to carry out the assay steps of the invention are known in the art. Generally, any reliable method of detecting specific proteins or mRNAs can be adapted. Preference is given to techniques based on desirable characteristics, such as, the ability to assay large numbers of samples and/or provide results quickly or that the assay is inexpensive to practice, or some optimum of these parameters. Tumor tissue to assay can be obtained as bulk tissue through surgery or in cellular form from blood, bone marrow, cell aspirates, peritoneal lavage, plural aspirates, or bronchial washes, and the like.

The assaying step can include a determination of at least one of presence, absence, abundance or condition of a TuAA in the panel. In some embodiments, the determination includes analysis of at least one of: mRNA, peptide, polypeptide, mature protein, and MHC-peptide complex. In some embodiments, the determination includes analysis of at least one of: processing state of a polypeptide, differential splicing of a nucleic acid, mutation of a nucleic acid in comparison with a germline sequence, variation of a nucleic acid or polypeptide sequence from a consensus sequence in a population, cellular localization, subcellular localization, and co-expression with a marker.

Commonly, detection of specific proteins involves the use of antibodies. Immunohistochemistry (IHC) is broadly applicable, but western hybridization, radioimmunoassay (RIA), and flow cytometry can also be used; collectively protein determinations. TRC-tetramers and antibodies recognizing specific peptide-MHC complexes can also be used. Tumor tissue can be used as target or stimulator in a wide variety of immunological assays (Elispot, T cell hybridoma reactivity, microcytotoxicity, and the like). Such assays are specific for a target epitope, not just the parent antigen, and thus can be referred to as epitope determinations. Detection of specific mRNA can be accomplished using any of several modalities of RT-PCR (reverse transcription-polymerase chain reaction) and similar nucleic acid amplification techniques (e.g., 3SR), northern hybridization, querrying of gene arrays with mRNA or cDNA, and in situ hybridization; collectively transcript determinations. Reagents that detect presentation of particular T cell epitopes from target antigens can also be used. These include, for example, T cell lines and hybridomas, and more preferably, antibodies specific for the peptide-MHC complex and TCR tetramers (see for example Li et al., Nature Biotech. 23:349-354, 2005, which is incorporated herein by reference in its entirety).

PCR techniques are sensitive and generally easy to implement, however, they cannot detect the mosaicism of antigen expression within a sample. IHC (and other in situ techniques), though potentially more labor intensive, allow spatial variation of expression within a sample to be observed. Thus, distinctions between co-expression of antigens within the same cells versus co-expression within different cells within the same sample can be made. Both situations can be desirable, the former providing for greater redundancy of targeting and reduced likelihood of antigen-loss escape mutants arising, the latter revealing how a greater proportion of the total tumor tissue can be directly targeted. Such information is also relevant to the use of antigens with more complex expression patterns. For example, PSMA, which can be expressed by prostate cells and tumor neovasculature, can be used as a prostate lineage marker if its expression can be associated specifically with the neoplastic cells, either through use of an in situ detection methodology or microdissection before assaying expression.

In preferred embodiments of the invention, the immunotherapeutic agent induces a T cell response, especially including a class I MHC-restricted T cell response. Thus, it can be advantageous to confirm MHC expression by the tumor tissue. Reagents for detection of MHC, including for PCR and antibody based methods, are widely known in the art. Class-, locus- and type-specific reagents are in common usage. Class I expression can also be assessed by detection of β2-microglobulin. Class- and locus-specific reagents offer the advantage of a broadly applicable uniform procedure. Type-specific reagents allow for simultaneous confirmation of expression and MHC type. Antibody-based techniques can offer the advantage of directly detecting protein expression at the cell surface, which is of clinical relevance, in contrast to RT-PCR and the like, from which surface expression can only be inferred. TCR tetramer-based assays allow simultaneous confirmation of both MHC and target antigen (indeed, even target epitope) expression and are inherently type specific.

Panel Design

Several modalities of the disclosed methods are within the scope of the invention. The first is concerned primarily with identifying antigens that are available to be targeted in a particular patient's tumor tissue. Thus, in some embodiments, the panel of antigens assayed for in practicing the methods disclosed herein is assembled from more commonly expressed TuAAs for which targeting immunotherapeutics are available (marketed or in development). Antigens can be included in a panel on a prospective basis, for example, due to common or highly specific expression in one or another subset of tumors, or in anticipation of the development of a corresponding therapeutic product. Thus, the same research observations that indicate an antigen would be a good target for immunotherapy (e.g., specificity, prevalence, and level of expression; presentation for T cell based products or surface expression for antibody based products; and that by inclusion in a multivalent immunotherapeutic redundancy or breadth of targeting can be increased) also can justify inclusion of that antigen in the diagnostic panels of the invention.

An antigen whose expression is specific to a particular tumor type, such as tyrosinase in melanoma, is suitable for panels used in screening that tumor type. An antigen that is expressed in a variety of tumor types, even if not highly prevalent in any particular one, can be suitable for inclusion in panels used to screen that variety of tumor types or in panels used as a general screen, e.g., not tied to an individual tumor type. In some embodiments, the panel of antigens specifically excludes markers from complex expression profiles associated with cancer, and the like, that are not appropriate targets of immunotherapy.

The histologic origin of a tumor is generally of clinical interest, for example, in designing a treatment strategy or confirming that an apparent recurrence is related to the presumptive original cancer. To this end, lineage markers can be included in the panels of antigens.

Marketing of Cancer Immunotherapeutics

Embodiments of the invention disclosed herein relate to methods for identifying patients that can benefit from particular cancer immunotherapies, which can also be useful in the identification of candidates for participation in clinical trails of such products and in marketing the vaccines. Much diagnostic work for cancer is carried out in centralized labs. Whether for recruitment or marketing, an arrangement is made with one or more of these laboratories. The arrangement can entail a fee-for-service contract or a partnership or joint venture. In an embodiment, the laboratory includes TuAA expression profiling assays, as described herein, in their standard panel of tests carried out on submitted tumor samples and the results are reported along with those of the other tests. When a tumor sample is identified as positive for one of more antigens corresponding to constituent immunogens of a vaccine, a notice is included in the same communication as the test report alerting the doctor (or patient if the results of the test are reported directly to the patient) to the availability of a clinical trial, for which the patient may be eligible or a product from which the patient may benefit.

Tumor Associated Antigens

Examples of TuAAs useful in embodiments disclosed herein include tyrosinase (SEQ. ID NO. 1), melan-A, (SEQ. ID NO. 2), SSX-2, (SEQ. ID NO.3), PSMA (prostate-specific membrane antigen) (SEQ. ID NO. 4), MAGE-1 (SEQ. ID NO. 5), MAGE-3 (SEQ. ID NO. 6), NY-ESO-1 (SEQ. ID NO. 7), PRAME (SEQ ID NO.8), Her2/Neu (SEQ ID NO. 9), PSA (SEQ ID NO. 10), C35 (SEQ ID NO. 11), SSX-4 (SEQ ID NO. 12), gp100 (SEQ ID NO. 13), thyroid transcription factor 1 (TTF1) (SEQ ID NO. 14), mammaglobin (SEQ ID NO. 15), prolactin-inducible protein (Brst2) (SEQ ID NO. 16), mesothelin, isoform 1 (SEQ ID NO. 17), mesothelin, isoform 2 (SEQ ID NO. 18), PSCA (SEQ ID NO. 19) SCP-1 (SEQ ID NO. 20), VEGF-A (SEQ ID NO. 21), and PLK1 (SEQ ID NO. 22). The natural coding sequences for these proteins, or any segments within them, can be determined from their cDNA or complete coding (cds) sequences, SEQ. ID NOS. 23-44, respectively. The sequences described in Table 1 are provided in the Sequence Listing filed herewith.

TABLE 1
SEQ. ID NOS.

| SEQ. ID | | |
|---|---|---|
| NO. | IDENTITY | ACCESSION NUMBER** |
| 1 | Tyrosinase protein | P14679 |
| 2 | Melan-A protein | Q16655 |
| 3 | SSX-2 protein | NP_003138 |
| 4 | PSMA protein | NP_004467 |
| 5 | MAGE-1 protein | P43355 |
| 6 | MAGE-3 protein | P43357 |
| 7 | NY-ESO-1 protein | P78358 |
| 8 | PRAME protein | NP_006106 |
| 9 | Her2/Neu protein | P04626 |
| 10 | PSA protein | NP_001639 |
| 11 | C35 protein | NP_115715 |
| 12 | SSX-4 protein | NP_783856 |
| 13 | gp100 protein | NP_008859 |
| 14 | TTF1 protein | NP_003308 |
| 15 | mammaglobin protein | NP_002402 |
| 16 | Brst2 protein | NP_002643 |
| 17 | Mesothelin, isoform 1, protein | NP_005814 |
| 18 | Mesothelin, isoform 2, protein | NP_037536 |
| 19 | PSCA protein | NP_005663 |
| 20 | SCP-1 protein | Q15431 |
| 21 | PLK1 protein | P53350 |
| 22 | VEGF-A protein | P15692 |
| 23 | Tyrosinase cDNA | NM_000372 |
| 24 | Melan-A cDNA | U06452 |
| 25 | SSX-2 cDNA | NM_003147 |
| 26 | PSMA cDNA | NM_004476 |
| 27 | MAGE-1 cds | M77481 |
| 28 | MAGE-3 cds | U03735 |
| 29 | NY-ESO-1 cDNA | U87459 |
| 30 | PRAME cDNA | NM_006115 |
| 31 | Her2/Neu cDNA | M11730 |
| 32 | PSA cDNA | NM_001648 |
| 33 | C35 cDNA | NM_032339 |
| 34 | SSX-4 cDNA | NM_175729 |
| 35 | gp100 cDNA | NM_006928 |
| 36 | TTF1 cDNA | NM_003317 |
| 37 | mammaglobin cDNA | NM_002411 |
| 38 | Brst2 cDNA | NM_002652 |
| 39 | Mesothelin, isoform 1, cDNA | NM_005823 |
| 40 | Mesothelin, isoform 2, cDNA | NM_013404 |
| 41 | PSCA cDNA | NM_005672 |
| 42 | SCP-1 cDNA | NM_003176 |
| 43 | VEGF-A cDNA | NM_001025366 |
| 44 | PLK1 cDNA | NM_005030 |

**All accession numbers used here and throughout can be accessed through the NCBI databases, for example, through the Entrez seek and retrieval system on the world wide web.

Tyrosinase is a melanin biosynthetic enzyme that is considered one of the most specific markers of melanocytic differentiation. Tyrosinase is expressed in few cell types, primarily in melanocytes, and high levels are often found in melanomas. The usefulness of tyrosinase as a TuAA is taught in U.S. Pat. No. 5,747,271, entitled “METHOD FOR IDENTIFYING INDIVIDUALS SUFFERING FROM A CELLULAR ABNORMALITY SOME OF WHOSE ABNORMAL CELLS PRESENT COMPLEXES OF HLA-A2/TYROSINASE DERIVED PEPTIDES, AND METHODS FOR TREATING SAID INDIVIDUALS,” which is hereby incorporated by reference in its entirety.

GP100, also known as PMel17, is another melanin biosynthetic protein expressed at high levels in melanomas. GP100 as a TuAA is disclosed in U.S. Pat. No. 5,844,075 entitled, “MELANOMA ANTIGENS AND THEIR USE IN DIAGNOSTIC AND THERAPEUTIC methods,” which is hereby incorporated by reference in its entirety.

Melan-A, also known as MART-1 (Melanoma Antigen Recognized by T cells), is another melanin biosynthetic protein expressed at high levels in melanomas. The usefulness of Melan-A/MART-1 as a TuAA is taught in U.S. Pat. Nos. 5,874,560 and 5,994,523, both entitled “MELANOMA ANTIGENS AND THEIR USE IN DIAGNOSTIC AND THERAPEUTIC METHODS,” as well as U.S. Pat. No. 5,620,886, entitled “ISOLATED NUCLEIC ACID SEQUENCE CODING FOR A TUMOR REJECTION ANTIGEN PRECURSOR PROCESSED TO AT LEAST ONE TUMOR REJECTION ANTIGEN PRESENTED BY HLA-A2,” each of which is hereby incorporated by reference in its entirety.

SSX-2, also known as Hom-Mel-40, is a member of a family of highly conserved cancer-testis (CT) antigens (Gure, A. O. et al., Int. J. Cancer 72:965-971, 1997, which is hereby incorporated by reference in its entirety). Its identification as a TuAA is taught in U.S. Pat. No. 6,025,191, entitled “ISOLATED NUCLEIC ACID MOLECULES WHICH ENCODE A MELANOMA SPECIFIC ANTIGEN AND USES THEREOF,” which is hereby incorporated by reference in its entirety. Cancer-testis antigens are found in a variety of tumors, but are generally absent from normal adult tissues except testis. Expression of different members of the SSX family has been found in various tumor cell lines. Due to the high degree of sequence identity among SSX family members, similar epitopes from more than one member of the family will be generated and able to bind to an MHC molecule, so that some vaccines directed against one member of this family can cross-react and be effective against other members of this family.

MAGE-1 (melanoma-associated antigen-1), MAGE-2 (melanoma-associated antigen-2), and MAGE-3 (melanoma-associated antigen-3) are members of another family of cancer-testis antigens originally discovered in melanoma but found in a variety of tumors. The identification of MAGE proteins as TuAAs is taught in U.S. Pat. No. 5,342,774, entitled “NUCLEOTIDE SEQUENCE ENCODING THE TUMOR REJECTION ANTIGEN PRECURSOR, MAGE-1,” which is hereby incorporated by reference in its entirety, and in numerous subsequent patents. Currently there are 17 entries for (human) MAGE in the SWISS Protein database. There is extensive similarity among these proteins, such that in many cases, an epitope from one can induce a cross-reactive response to other members of the family. A few members of the MAGE family have not been observed in tumors, most notably MAGE-H1 and MAGE-D1, which are expressed in testes and brain, and bone marrow stromal cells, respectively. The possibility of cross-reactivity on normal tissue is ameliorated by the fact that they are among the least similar to the other MAGE proteins.

GAGE-1 is a member of the GAGE family of cancer testis antigens (Van den Eynde, B., et al., J. Exp. Med. 182: 689-698, 1995; U.S. Pat. Nos. 5,610,013; 5,648,226; 5,858,689; 6,013,481; and 6,069,001, each of which is hereby incorporated by reference in its entirety). The PubGene database currently lists 12 distinct accessible members, some of which are synonymously known as PAGE or XAGE. GAGE-1 through GAGE-8 have a very high degree of sequence identity, so most epitopes can be shared among multiple members of the family.

BAGE is a cancer-testis antigen commonly expressed in melanoma, particularly metastatic melanoma, as well as in carcinomas of the lung, breast, bladder, and squamous cells of the head and neck. Its usefulness as a TuAA is taught in U.S. Pat. No. 5,683,88, entitled “TUMOR REJECTION ANTIGENS WHICH CORRESPOND TO AMINO ACID SEQUENCES IN TUMOR REJECTION ANTIGEN PRECURSOR BAGE, AND USES THEREOF” and U.S. Pat. No. 5,571,711, entitled “ISOLATED NUCLEIC ACID MOLECULES CODING FOR BAGE TUMOR REJECTION ANTIGEN PRECURSORS,” each of which is hereby incorporated by reference in its entirety.

NY-ESO-1, also known as CTAG-1 (Cancer-Testis Antigen-1) and CAG-3 (Cancer Antigen-3), is a cancer-testis antigen found in a wide variety of tumors. NY-ESO-1 as a TuAA is disclosed in U.S. Pat. No. 5,804,381, entitled “ISOLATED NUCLEIC ACID MOLECULE ENCODING AN ESOPHAGEAL CANCER ASSOCIATED ANTIGEN, THE ANTIGEN ITSELF, AND USES THEREOF,” which is hereby incorporated by reference in its entirety. A paralogous locus encoding antigens with extensive sequence identity, LAGE-1a/s and LAGE-1b/L, has been disclosed in publicly available assemblies of the human genome, and has been concluded to arise through alternate splicing. Additionally, CT-2 (or CTAG-2, Cancer-Testis Antigen-2) appears to be either an allele, a mutant, or a sequencing discrepancy of LAGE-1b/L. Due to the extensive sequence identity, many epitopes from NY-ESO-1 can also induce immunity to tumors expressing these other antigens. NY-ESO-1 and LAGE are virtually identical through amino acid 70. From amino acid 71 through 134 the longest run of identity between the two proteins is 6 residues, but potentially cross-reactive sequences are present. From amino acid 135 through 180, NY-ESO and LAGE-1a/s are identical except for a single residue, but LAGE-1b/L is unrelated due to the alternate splice. The CAMEL and LAGE-2 antigens appear to derive from the LAGE-1 mRNA, but from alternate reading frames, thus giving rise to unrelated protein sequences. More recently, GenBank Accession AF277315.5, Homo sapiens chromosome X clone RP5-865E18, RP5-1087L19, complete sequence, reports three independent loci in this region which are labeled as LAGE1 (corresponding to CTAG-2 in the genome assemblies), LAGE2-A and LAGE2-B (both corresponding to CTAG-1 in the genome assemblies).

PRAME, also known as MAPE, DAGE, and OIP4, was originally observed as a melanoma antigen. Subsequently, it has been recognized as a cancer-testis (CT) antigen, but unlike many CT antigens, such as, MAGE, GAGE and BAGE, PRAME is expressed in acute myeloid leukemias. PRAME is a member of the MAPE family, which consists largely of hypothetical proteins with which it shares limited sequence similarity. The usefulness of PRAME as a TuAA is taught in U.S. Pat. No. 5,830,753, entitled “ISOLATED NUCLEIC ACID MOLECULES CODING FOR TUMOR REJECTION ANTIGEN PRECURSOR DAGE AND USES THEREOF,” which is hereby incorporated by reference in its entirety.

PSMA (prostate-specific membranes antigen), a TuAA described in U.S. Pat. No. 5,538,866, entitled “PROSTATE-SPECIFIC MEMBRANES ANTIGEN,” which is hereby incorporated by reference in its entirety, is expressed by normal prostate epithelium and, at a higher level, in prostatic cancer. Additionally expression has also been observed in ovarian carcinoma. It has also been found in the neovasculature of non-prostatic tumors. PSMA can thus form the basis for vaccines directed to both prostate and ovarian cancer and to the neovasculature of other tumors. This later concept is more fully described in a U.S. Provisional Patent Application No. 60/274,063, filed Mar. 7, 2001, and U.S. patent application Ser. No. 10/094,699 (Pub. No. 2003-0046714 A1), filed on Mar. 7, 2002, Ser. No. 11/073,347 (Pub. No. ______), filed Jun. 30, 2005, each entitled “ANTI-NEOVASCULATURE PREPARATIONS FOR CANCER,” each of which is hereby incorporated by reference in its entirety. Briefly, as tumors grow they recruit ingrowth of new blood vessels. This is understood to be necessary to sustain growth as the centers of unvascularized tumors are generally necrotic and angiogenesis inhibitors have been reported to cause tumor regression. Such new blood vessels, or neovasculature, express antigens not found in established vessels, and thus can be specifically targeted. By inducing CTL against neovascular antigens the vessels can be disrupted, interrupting the flow of nutrients to, and removal of wastes from, tumors, leading to regression.

Alternate splicing of the PSMA mRNA leads to a protein with an apparent start at Met₅₈, thereby deleting the putative membrane anchor region of PSMA as described in U.S. Pat. No. 5,935,818, entitled “ISOLATED NUCLEIC ACID MOLECULE ENCODING ALTERNATIVELY SPLICED PROSTATE-SPECIFIC MEMBRANES ANTIGEN AND USES THEREOF,” which is hereby incorporated by reference in its entirety. A protein termed PSMA-like protein, Genbank accession number AF261715, is nearly identical to amino acids 309-750 of PSMA, but has a different expression profile. Thus, the most preferred epitopes are those with an N-terminus located from amino acid 58 to 308.

PSA (prostate specific antigen) is a peptidase of the kallikrein family and a differentiation antigen of the prostate. Expression in breast tissue has also been reported. Alternate names include gamma-seminoprotein, kallikrein 3, seminogelase, seminin, and P-30 antigen. PSA has a high degree of sequence identity with the various alternate splicing products prostatic/glandular kallikrein-1 and -2, as well as kalikrein 4, which is also expressed in prostate and breast tissue. Other kallikreins generally share less sequence identity and have different expression profiles. Nonetheless, cross-reactivity that might be provoked by any particular epitope, along with the likelihood that that epitope would be liberated by processing in non-target tissues (most generally by the housekeeping proteasome), should be considered in designing a vaccine.

PSCA (prostate stem cell antigen) and also known as SCAH-2, is a differentiation antigen preferentially expressed in prostate epithelial cells, and overexpresssed in prostate cancers. Lower level expression is seen in some normal tissues including neuroendocrine cells of the digestive tract and collecting ducts of the kidney. PSCA is described in U.S. Pat. No. 5,856,136, entitled “HUMAN STEM CELL ANTIGENS,” which is hereby incorporated by reference in its entirety.

Synaptonemal complex protein 1 (SCP-1), also known as HOM-TES-14, is a meiosis-associated protein and also a cancer-testis antigen (Tureci, O., et al. Proc. Natl. Acad. Sci. USA 95:5211-5216, 1998, which is hereby incorporated by reference in its entirety). As a cancer antigen its expression is not cell-cycle regulated and it is found frequently in gliomas, breast, renal cell, and ovarian carcinomas. It has some similarity to myosins, but with few enough identities that cross-reactive epitopes are not an immediate prospect.

The ED-B domain of fibronectin is also a potential target. Fibronectin is subject to developmentally regulated alternative splicing, with the ED-B domain being encoded by a single exon that is used primarily in oncofetal tissues (Matsuura, H. and S. Hakomori Proc. Natl. Acad. Sci. USA 82:6517-6521, 1985; Carnemolla, B. et al., J. Cell Biol. 108:1139-1148, 1989; Loridon-Rosa, B. et al., Cancer Res. 50: 1608-1612, 1990; Nicolo, G. et al., Cell Differ. Dev. 32:401-408, 1990; Borsi, L. et al., Exp. Cell Res. 199:98-105, 1992; Oyama, F. et al., Cancer Res. 53:2005-2011, 1993; Mandel, U. et al., APMIS 102:695-702, 1994; Farnoud, M. R. et al., Int. J. Cancer 61:27-34, 1995; Pujuguet, P. et al., Am. J. Pathol. 148:579-592, 1996; Gabler, U. et al., Heart 75:358-362, 1996; Chevalier, X., Br. J. Rheumatol. 35:407-415, 1996; Midulla, M., Cancer Res. 60:164-169, 2000, each of which is hereby incorporated by reference in its entirety).

The ED-B domain is also expressed in fibronectin of the neovasculature (Kaczmarek, J. et al., Int. J. Cancer 59:11-16, 1994; Castellani, P. et al., Int. J. Cancer 59:612-618, 1994; Neri, D. et al., Nat. Biotech. 15:1271-1275, 1997; Karelina, T. V. and A. Z. Eisen Cancer Detect. Prev. 22:438-444, 1998; Tarli, L. et al., Blood 94:192-198, 1999; Castellani, P. et al., Acta Neurochir. (Wien) 142:277-282, 2000, each of which is hereby incorporated by reference in its entirety). As an oncofetal domain, the ED-B domain is commonly found in the fibronectin expressed by neoplastic cells in addition to being expressed by the neovasculature. Thus, CTL-inducing vaccines targeting the ED-B domain can exhibit two mechanisms of action: direct lysis of tumor cells, and disruption of the tumor's blood supply through destruction of the tumor-associated neovasculature. As CTL activity can decay rapidly after withdrawal of vaccine, interference with normal angiogenesis can be minimal. The design and testing of vaccines targeted to neovasculature is described in U.S. Provisional Patent Application No. 60/274,063, filed on Mar. 7, 2001, and in U.S. patent application Ser. No. 10/094,699, (Pub. No. 2003-0046714 A1), filed Mar. 7, 2002, Ser. No. 11/073,347 (Pub. No. ______), filed Jun. 30, 2005, all entitled “ANTI-NEOVASCULATURE PREPARATIONS FOR CANCER,” filed on Mar. 7, 2002, each of which is hereby incorporated by reference in its entirety. A tumor cell line is disclosed in Provisional U.S. Application No. 60/363,131, filed on Mar. 7, 2002, entitled “HLA-TRANSGENIC MURINE TUMOR CELL LINE,” which is hereby incorporated by reference in its entirety.

Carcinoembryonic antigen (CEA) is a paradigmatic oncofetal protein first described in 1965 (Gold and Freedman, J. Exp. Med. 121: 439-462, 1965, which is hereby incorporated by reference in its entirety). Fuller references can be found in the Online Mendelian Inheritance in Man; record *114890. It has officially been renamed carcinoembryonic antigen-related cell adhesion molecule 5 (CEACAM5). Its expression is most strongly associated with adenocarcinomas of the epithelial lining of the digestive tract and in fetal colon. CEA is a member of the immunoglobulin supergene family and the defining member of the CEA subfamily.

Survivin, also known as Baculoviral IAP Repeat-Containing Protein 5 (BIRC5), is another protein with an oncofetal pattern of expression. It is a member of the inhibitor of apoptosis protein (IAP) gene family. It is widely over-expressed in cancers (Ambrosini, G. et al., Nat. Med. 3:917-921, 1997; Velculiscu V. E. et al., Nat. Genet. 23:387-388, 1999, which is hereby incorporated by reference in its entirety) and its function as an inhibitor of apoptosis is believed to contribute to the malignant phenotype.

HER2/NEU is an oncogene related to the epidermal growth factor receptor (van de Vijver, et al., New Eng. J. Med. 319:1239-1245, 1988, which is hereby incorporated by reference in its entirety), and apparently identical to the c-ERBB2 oncogene (Di Fiore, et al., Science 237: 178-182, 1987, which is hereby incorporated by reference in its entirety). The over-expression of ERBB2 has been implicated in the neoplastic transformation of prostate cancer. As with HER2, it is amplified and over-expressed in 25-30% of breast cancers among other tumors where expression level is correlated with the aggressiveness of the tumor (Slamon, et al., New Eng J. Med. 344:783-792, 2001, which is hereby incorporated by reference in its entirety). A more detailed description is available in the Online Mendelian Inheritance in Man; record *164870.

MESOTHELIN is an antigen originally found in mesotheliomas but also known to be upregulated in many pancreatic and ovarian cancers. Its use as a vaccine target and useful epitopes are described in Thomas, A. M. et al., J. Exp. Med. 200:297-306, 2004, which is hereby incorporated by reference in its entirety.

Vascular Endothelial Growth Factor (VEGF-A or VEGF) is a mitogenic protein structurally related to platelet-derived growth factor, but with narrower mitogenic activity focused on vascular endothelial cells. The protein and its receptors are important to tumor growth and their potential as targets of cancer therapies has been noted (Folkman, J. Nature Med. 1: 27-31, 1995, which is hereby incorporated by reference in its entirety). A more detailed description is available in the Online Mendelian Inheritance in Man; record *192240.

PLK1 is an intracellular serine/threonine kinase that plays a critical role in cell cycle regulation and in DNA damage responses. Mutation or knock down of PLK1 result in abnormal mitosis, cell cycle arrest and apoptosis (Reagan-Shaw S. and Ahmad N. FASEB J. 19:611, 2005). PLK1 belongs to the Polo-like kinase family of structurally conserved kinases. The PLK1 family contains two conserved regions, an N-terminal kinase domain and a C-terminal non-catalytic Polo box region (Lowery D M et al., Oncogene 24:248.2005) responsible for subcellular localization. PLK1 is predominantly found in the cytoplasm during interphase and localizes to the nucleus during mitosis (Takai N et al., Oncogene 24:2872005). Nuclear localization of PLK1 is essential for its biological function (Lee K S et al., Proc Natl Acad Sci USA 95:9301, 1998). While most classical tumor associated antigens are not known to participate in tumor initiation or disease progression, PLK1 has been shown to drive tumor growth. Thus, in some embodiments, PLK1 can be considered a tumor-associated antigen that can be used as a target for cancer immunotherapy. Based on its expression profile, PLK1 is expressed in proliferating cells, with elevated expression found in most solid tumors. This expression pattern renders PLK1 a valid target for active and passive immunotherapy.

Further examples of tumor-associated antigens include MelanA (MART-I), gp100 (Pmel 17), tyrosinase, TRP-1, TRP-2, MAGE-1, MAGE-3, BAGE, GAGE-1, GAGE-2, p15(58), CEA, RAGE, NY-ESO (LAGE), SCP-1, Hom/Mel-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, β-Catenin, CDK4, Mum-1, p16, TAGE, PSMA, PSCA, CT7, telomerase, 43-9F, 5T4, 791Tgp72, alpha-fetoprotein, β-HCG, BCA225, BTAA, CA 125, CA 15-3 (CA 27.29\BCAA), CA 195, CA 242, CA-50, CAM43, CD68\KP1, CO-029, FGF-5, G250, Ga733 (EpCAM), HTgp-175, M344, MA-50, MG7-Ag, MOV18, NB/70K, NY-CO-1, RCAS1, SDCCAG16, TA-90 (Mac-2 binding protein\cyclophilin C-associated protein), TAAL6, TAG72, TLP, TPS, and the like.

Additional tumor-associated antigens are described in Chen, Y T, “Identification of human tumor antigens by serological expression cloning: an online review on SEREX” Cancer Immun. 2004 [updated 2004 Mar. 10; cited 2004 Apr. 1] at world wide web cancerimmunotherapy.org/SEREX/; and Renkvist, N. et al., “A listing of tumor antigens recognized by T cells,” Cancer Immunology Immunotherapy, 50:3-15 (2001), each of which is hereby incorporated by reference in its entirety.

Table 2, adapted from Scanlan et al., “The cancer/testis genes: Review, standardization, and commentary,” Cancer Immunity 4:1 (Jan. 23, 2004), which is hereby incorporated by reference in its entirety, provides a listing of CT Antigens. Table 3 provides the frequency of mRNA expression in various tumor types for the CT antigens in Table 2. Scanlan et al., “The cancer/testis genes: Review, standardization, and commentary,” Cancer Immunity 4:1 (Jan. 23, 2004), which is hereby incorporated by reference in its entirety.

TABLE 2
Listing of CT genes

| CT | Transcript/Transcript | |
|---|---|---|
| Identifier | family | Family Members/CT Identifier (Synonyms) |
| CT1 | MAGEA | MAGEA1/CT1.1, MAGEA2/CT1.2, |
| | | MAGEA3/CT1.3, MAGEA4/CT1.4, |
| | | MAGEA5/CT1.5, MAGEA6/CT1.6, |
| | | MAGEA7/CT1.7, MAGEA8/CT1.8, |
| | | MAGEA9/CT.9, MAGEA10/CT1.10, |
| | | MAGEA11/CT1.11, MAGEA12/CT1.12 |
| CT2 | BAGE | BAGE/CT2.1, BAGE2/CT2.2, BAGE3/CT2.3, |
| | | BAGE4/CT2.4, BAGE5/CT2.5 |
| CT3 | MAGEB | MAGEB1/CT3.1, MAGEB2/CT3.2, |
| | | MAGEB5/CT3.3, MAGEB6/CT3.4 |
| CT4 | GAGE1 | GAGE1/CT4.1, GAGE2/CT4.2, GAGE3/CT4.3, |
| | | GAGE4/CT4.4, GAGE5/CT4.5, GAGE6/CT4.6, |
| | | GAGE7/CT4.7, GAGE8/CT4.8 |
| CT5 | SSX | SSX1/CT5.1, SSX2/CT5.2a, SSX2/CT5.2b, |
| | | SSX3/CT5.3, SSX4/CT5.4 |
| CT6 | NY-ESO-1 | NY-ESO-1/CT6.1, LAGE-1a/CT6.2a, LAGE |
| | | 1b/CT6.2b |
| CT7 | MAGEC1 | MAGEC1/CT7.1, MAGEC3/CT7.2 |
| CT8 | SYCP1 | SYCP1/CT8 |
| CT9 | BRDT | BRDT/CT9 |
| CT10 | MAGEE1 | MAGEE1/CT10 |
| CT11 | CTp11/SPANX | SPANXA1/CT11.1, SPANXB1/CT11.2, |
| | | SPANXC/CT11.3, SPANXD/CT11.4 |
| CT12 | XAGE-1/GAGED | XAGE-1a/CT12.1a, XAGE-1b/CT12.1b, XAGE- |
| | | 1c/CT12.1c, XAGE-1d/CT12.1d, XAGE-2/CT12.2, |
| | | XAGE-3a/CT12.3a, XAGE-3b/CT12.3b, XAGE- |
| | | 4/CT12.4 |
| CT13 | HAGE | HAGE/CT13 |
| CT14 | SAGE | SAGE/CT14 |
| CT15 | ADAM2 | ADAM2/CT15 |
| CT16 | PAGE-5 | PAGE-5/CT16.1, C16.2 |
| CT17 | LIP1 | LIP1/CT17 |
| CT18 | NA88 | NA88/CT12 |
| CT19 | IL13RA1 | IL13RA1/CT19 |
| CT20 | TSP50 | TSP50/CT20 |
| CT21 | CTAGE-1 | CTAGE-1/CT21.1, CTAGE-2/CT21.2 |
| CT22 | SPA17 | SPA17/CT22 |
| CT23 | OY-TES-1 | OY-TES-1/CT23 |
| CT24 | CSAGE | CSAGE/CT24.1, TRAG3/CT24.2 |
| CT25 | MMA1/DSCR8 | MMA-1a/CT25.1a, MMA-1b/CT25.1b |
| CT26 | CAGE | CAGE/CT26 |
| CT27 | BORIS | BORIS/CT27 |
| CT28 | HOM-TES-85 | HOM-TES-85/CT28 |
| CT29 | AF15q14/D40 | D40/CT29 |
| CT30 | E2F-like/HCA661 | HCA661/CT30 |
| CT31 | PLU-1 | PLU-1/CT31 |
| CT32 | LDHC | LDHC/CT32 |
| CT33 | MORC | MORC/CT33 |
| CT34 | SGY-1 | SGY-1/CT34 |
| CT35 | SPO11 | SPO11/CT35 |
| CT36 | TPX1 | TPX-1/CT36 |
| CT37 | NY-SAR-35 | NY-SAR-35/CT37 |
| CT38 | FTHL17 | FTHL17/CT38 |
| CT39 | NXF2 | NXF2/CT39 |
| CT40 | TAF7L | TAF7L/CT40 |
| CT41 | TDRD1 | TDRD1/CT41.1, NY-CO-45/CT41.2 |
| CT42 | TEX15 | TEX15/CT42 |
| CT43 | FATE | FATE/CT43 |
| CT44 | TPTE | TPTE/CT44 |
| — | PRAME | (MAPE, DAGE) |

TABLE 3

| | Frequency (%) of Expression in Tumor Type | | | | | | | | | | | | | | | | |
|---|---|---|---|---|---|---|---|---|---|---|---|---|---|---|---|---|---|
| | | | | | | | | | Leuk/ | Lung | | | | | | | |
| CT Family (Member) | Blad | Brn | Brst | Col | Eso | Gas | H/N | Liver | Lymph | (NSCLC) | Mel | Ov | Pancr | Pros | Renal | Sarc | Ref |
| MAGEA1/CT1.1 | 22 | — | 18 | 2 | 53 | 29 | 28 | 80 | 0 | 49 | 48 | 28 | — | 15 | 0 | 14 | 44 |
| BAGE1/CT2.1 | 15 | — | 10 | 0 | — | — | 8 | — | 0 | 4 | 26 | 15 | — | 0 | 0 | 6 | 44 |
| MAGEB1/CT3.1 | 0 | 0 | 17 | 0 | — | 0 | 0 | — | 0 | 14 | 22 | — | — | 0 | 0 | 9 | 45 |
| GAGE/CT4.1 | 12 | — | 9 | 0 | — | — | 19 | 38^{b} | 1 | 19 | 28 | 31 | — | 10 | 0 | 25 | 44 |
| SSX2/CT5.2 | 44 | 6 | 7 | 12 | — | — | 35 | 9^{b} | 36 | 16 | 35 | — | — | 40 | 5 | 50 | 46 |
| NY-ESO-1/CT6.1 | 80 | 0 | 30 | 0 | — | 0 | — | 29 | 0 | 17 | 34 | 25 | 0 | 25 | 9 | 0 | 8 |
| MAGEC1/CT7.1 | 44 | — | 30 | 10 | — | — | 36 | — | — | 33 | 70 | — | — | — | — | 60 | 20 |
| SYCP1/CT8 | — | 47 | 20 | 0 | — | 7 | — | 28^{b} | 0 | 7 | 14 | 0 | — | 0 | 8 | 0 | 9 |
| BRDT/CT9 | 0 | — | 0 | 0 | 8 | — | 8 | — | — | 25 | 0 | — | — | — | 0 | — | 16 |
| MAGEE1/CT10 | 44 | — | 38 | 0 | — | — | 36 | — | — | 24 | 50 | — | — | — | — | 0 | 12 |
| SPANXC/CT11.3 | 9 | — | 25 | 22 | 0 | — | — | — | — | 33 | 70 | — | 0 | — | — | — | 14 |
| XAGE-1a/CT12.1a | — | — | — | — | — | — | — | — | — | — | 8 | — | — | — | — | 22 | 47 |
| HAGE/CT13 | 24 | 37 | 5 | 31 | 27 | — | — | 20 | 9 | 32 | 17 | — | — | 22 | 6 | 20 | 13 |
| SAGE/CT14 | 12 | 0 | 5 | 0 | 20 | — | 17 | — | 4 | 22 | 4 | — | — | 0 | 5 | 5 | 13 |
| ADAM2/CT15 | — | — | 0 | 0 | — | — | — | — | — | 0 | 0 | 0 | — | — | 12 | — | 17 |
| PAGE-5/CT16 | — | — | 5 | 11 | — | — | — | — | — | 39 | 22 | 0 | — | — | 44 | — | 17 |
| LIP1/CT17 | — | — | 5 | 0 | — | — | — | — | — | 0 | 0 | 0 | — | — | 25 | — | 17 |
| NA88/CT18 | — | — | — | — | — | — | — | — | — | — | 11 | — | — | — | — | — | 48 |
| TSP50/CT20 | — | — | 28 | — | — | — | — | — | — | — | — | — | — | — | — | — | 49 |
| CTAGE-1/CT21.1 | — | — | — | — | — | — | — | — | 35 | — | — | — | — | — | — | — | 50 |
| SPA17/CT22 | — | — | — | — | — | — | — | — | 26 | — | — | — | — | — | — | — | 51 |
| OYTES1/CT23 | 28 | — | 40 | 15 | — | 0 | — | 40 | — | 20 | — | — | — | — | 0 | — | 52 |
| MMA1a/CT25.1a | — | — | 0 | 0 | 0 | — | — | — | — | 40 | 26 | — | 0 | — | — | 18 | 15 |
| CAGE/CT26 | — | — | — | — | — | 89 | — | — | — | 100 | — | — | — | — | — | — | 53 |
| HOMTES85/CT28 | — | 35 | 0 | 10 | — | — | — | 19 | — | 28 | 36 | 32 | — | 0 | — | — | 54 |
| D40/CT29 | — | 20 | — | 13 | — | 0 | — | — | — | 41 | — | 36 | 27 | — | — | — | 55 |
| HCA661/CT30 | 0 | — | — | — | — | 0 | 0 | 29 | — | — | 20 | — | — | — | — | — | 56 |
| PLU-1/CT31 | — | — | 86 | — | — | — | — | — | — | — | — | — | — | — | — | — | 27 |
| LDHC/CT32 | — | — | 35 | 15 | — | — | — | — | — | 47 | 44 | 42 | — | 37 | 57 | — | 18 |
| MORC/CT33 | — | — | 0 | 0 | — | — | — | — | — | 18 | 18 | 14 | — | 0 | 0 | — | 18 |
| SGY-1/CT34 | — | — | 20 | 0 | — | — | — | — | — | 12 | 25 | 57 | — | 12 | 0 | — | 18 |
| SPO11/CT35 | — | — | 0 | 0 | — | — | — | — | — | 0 | 6 | 0 | — | 0 | 0 | — | 18 |
| TPX1/CT36 | — | — | 15 | 0 | — | — | — | — | — | — | 6 | 14 | — | 37 | 14 | — | 18 |
| NYSAR35/CT37 | 42 | — | 23 | 0 | 8 | — | — | — | — | 17 | 6 | 8 | — | — | 0 | 8 | 57 |
| FTHL17/CT38 | 22 | — | 14 | 0 | 0 | — | 10 | — | 0 | 25 | 0 | — | — | 0 | 0 | 0 | 58 |
| NXF2/CT39 | 19 | — | 0 | 11 | 12 | — | 5 | — | 0 | 15 | 55 | — | — | 14 | 0 | 27 | 58 |
| TAF7L/CT40 | 10 | — | 0 | 0 | 0 | — | 10 | — | 0 | 9 | 21 | — | — | 0 | 0 | 12 | 58 |
| TDRD1/CT41.1 | 28 | — | 37 | 0 | 10 | — | 22 | — | 5 | 5 | 0 | — | — | 38 | 0 | 0 | 58 |
| TEX15/CT42 | 21 | — | 0 | 0 | 20 | — | 11 | — | 0 | 21 | 27 | — | — | 12 | 33 | 28 | 58 |
| FATE/CT43 | — | — | — | 21 | — | 7 | — | 66 | — | 0 | — | — | — | — | — | — | 19 |
| TPTE/CT44 | — | — | — | 0 | — | 0 | — | 39 | — | 36 | — | — | — | — | — | — | 19 |

^{a}Abbreviations: Blad, bladder; Brn, brain; Brst, breast; Col, colon; Gas, gastric; H/N, head and neck; Leuk, leukemia; Lymph, lymphoma, NSCLC, non-small cell lung carcinoma; Mel, melanoma; Ov, ovarian; Pancr, pancreatic; Pros, prostate; Sarc, sarcoma; Ref, reference.
^{b}Reference 59.

Many antigens listed in the tables above have no documented role in maintaining the transformed phenotype of a cell, and thus, a transformed cell may lose expression of such antigens without affecting the viability of the cell or the malignancy of the disease. Although some CT antigens, such as the SSX proteins, are known to be transcriptional regulators, their role, if any, in tumorigenicity remains obscure. It has also recently emerged that PRAME can repress signaling through the retinoic acid receptor to inhibit retinoic acid-induced differentiation, growth arrest, and apoptosis, thereby suggesting that PRAME over-expression can impart tumor cells with a growth or survival advantage. It would be advantageous to target an antigen, the loss of which necessarily would affect viability of the cell or the malignancy of the disease. Such antigens are included in preferred embodiments of the combinations of tumor and/or tumor and stromal antigens disclosed herein. However, many genes involved in the regulation of proliferation are important in oncogenesis primarily in mutated form so that specific immune attack on the mutated form depends on the mutation being appropriately placed within an epitope. Furthermore, epitopes present in the wild type molecule (whether or not the targeted molecule is wild type or mutant) will generally only be considered if there is a substantial difference in expression level between transformed and normal cells, or at least the normal cells of vital organs. Thus appropriate antigens have been difficult to recognize.

The expression of PLK1 is regulated during cell cycle progression as well as throughout the various disease stages. PLK1 1 expression is minimal during the initial phases of cell division, begins to increase during G2 and peaks at M phase. PLK1 is targeted for degradation by the proteasome pathway after cells exit from mitosis. In normal tissues PLK1 is expressed in adult organs containing highly proliferative cells, such as in the spleen, placenta ovary and testis. Its expression is undetected in vital organs such as heart, lung, liver, brain, intestines, smooth muscle and skin. Wild type PLK1 is overexpressed in tumor tissues, including breast, prostate, ovarian, non-small cell lung, head/neck, colon, pancreatic, endometrial and esophageal carcinomas (Table 4). Importantly, the level of PLK1 expression often correlates with more advanced stages of tumor progression and poor prognosis (Wolf G et al., Oncogene 14:543, 1997; Takai N et al., Cancer Lett. 164:41, 2001). This has been specifically demonstrated in the case of NSCLC, esophageal, and head/neck carcinomas. Thus, PLK1 is a viable target for cancer immunotherapy for those tumors in which it is overexpressed.

TABLE 4
Plk-1 Expression Profile

| | | Expression | |
|---|---|---|---|
| Tumor | Penetrance | Level | Reference |
| Lung | >90% | Med/strong | Wolf G et al., Oncogene |
| (NSCLC) | | | 14: 543, 1997. |
| Ovarian | >85% | Low/strong | Takai N et al., Cancer Lett. |
| | | | 164: 41, 2001 |
| Breast | 43% | Med/strong | Weichert W et al., |
| | | | Virchows Arch, 446: 442, |
| | | | 2005. |
| Prostate | 53% | Med/strong | Weichert W et al., |
| | | | Curr. Biol. 101: 4419, 2004. |
| Colorectal | 73% | Med/strong | Takahashi T et al., |
| | | | Cancer Sci. 94: 148 2003. |
| Pancreatic | 48% | Med/strong | Gray JP et al., Mol Cancer |
| | | | Ther. 3: 641, 2004. |
| Head & Neck | 72% | Med/strong | Knecht R et al., Cancer Res. |
| | | | 59: 2794, 1999. |
| Melanoma | 53% | Med | Strebhardt K et al., JAMA. |
| | | | 283: 479, 2000. |
| Esophageal | 96% | Med | Tokumitsu Y et al., Int J |
| | | | Oncol. 15: 687, 1999. |

Additional antigens associated with tumor neovasculature include VEGFR2 (vascular endothelial growth factor receptor 2) described in U.S. Pat. No. 6,342,221, which is hereby incorporated by reference in its entirety; and Tie-2, an endothelium specific receptor tyrosine kinase, which is described in WO 99/43801, and which is hereby incorporated by reference in its entirety.

In addition to the disruption of blood flow to tumors that can be achieved using anti-neovasculature agents such as those recited above, co-targeting molecules expressed on cancer cells as well as molecules expressed on underlying non-transformed stromal cells (including neovasculature as well as interstitial tissue, for example) can also improve the effectiveness of multivalent immunotherapeutics in limiting tumor growth and promoting cancer regression by other mechanisms. Stroma encompasses neovasculature as well as fibroblasts, and in general, all non-transformed, non-lymphoid cells within a tumor microenvironment. For example, immune mediated attack of the endothelial cells (via cytotoxic T lymphocytes (CTLs) or antibody dependent cytotoxic cells (ADCC)) can result in neovasculature permeabilization and initiation of inflammatory events that result in recruitment and translocation of immune effectors, such as CTLs, targeting the neoplastic cells within primary tumor and metastatic lesions. Moreover, as attacks based for example, on T cell recognition of endothelial cell MHC-peptide complexes occur in the luminal environment, any immune suppressive influence of the tumoral environment is minimized. Compared to strategies targeting only cancer cells, methods to co-target associated stromal tissue improve the efficacy of the former. In some embodiments, the efficacy is synergistically enhanced. Similarly, compared to strategies targeting neovasculature only, methods to co-target cancer cells improve the overall therapeutic effect by attacking lesions, including those of limited size and vascularization, especially those adversely located within vital organs. With regard to neovasculature, co-targeting VEGFRs (such as II), CD55 and PSMA as well as other molecules expressed by neovasculature, can be accomplished by generating CTL or antibodies with capability to initiate ADCC or complement activated cell injury. Alternatively, initial endothelial injury can be brought about though passive immunotherapy using available anti-angiogenic antibodies.

In addition or alternatively, co-targeting target-associated antigens, together with growth, metastasis, or survival promoting factors produced by cancer cells or non-transformed cells that are found in the extracellular compartment (diffusing or associated with the extracellular matrix), can also result in a more substantial therapeutic effect. By co-targeting antigens expressed within or on cancer cells as well as factors that exert autocrine or paracrine effects (growth, survival, and/or invasiveness), the pathogenic process can be slowed or disrupted to a significant degree; Co-targeting autocrine or paracrine factors (such as, but not limited to, NF-κB activating molecules—CXCL1, CXCL8, CCL2; or growth factors such as, but not limited to, chorionic-gonadotropic hormone, gastrin, and VEGF-A) can be carried out by co-induction of neutralizing antibodies or secondarily, by CTLs recognizing cells that produce such factors.

The interaction between transformed and stromal cells is mediated by VEGF-A. VEGF-A has been shown to play a key role in the establishment and functionality of tumor neovasculature; thus, deprival of VEGF-A by using specific passive immunotherapy (via anti-VEGF-A antibodies such as bevacizumab (AVASTIN®) resulted in control of tumor progression and metastatic disease for colorectal, lung, breast, ovarian carcinoma and other cancers. The mechanism of action likely involves direct neutralization of VEGF-A, thereby slowing down the establishment and progression of neovasculature. Neutralization of VEGF-A by passive immunotherapy can be combined with the active immunotherapies disclosed herein. VEGF-A can also be used as the target antigen for active or T-cell based immunotherapy directed against cells that produce VEGF-A in excess (most likely a subpopulation of tumoral cells, and most often the transformed cancer cells within the tumor environment). Such a strategy can be more effective in controlling a tumoral process both by mediating immune damage of cancer cells that express VEGF-A and by depriving the tumor neovasculature of an essential growth factor.

Overall, co-targeting multiple elements of biological importance for tumor growth and metastasis can limit progression of the malignant process by impacting the processes of clonal selection, immune evasion and escape. Thus, co-targeting stroma-associated antigens provides an additional mode of attack in that such activities are inhibited and/or disrupted.

One of skill in the art will appreciate that any other antigen or protein associated with vascular or other tumor-associated stromal cells can be a target for the immunogenic compositions, including those that are presently known and those yet to be identified.

Compositions

Immunogenic compositions, including, for example, vaccines, can be prepared using whole antigen or an epitopic peptide. Peptide immunogens can be readily prepared using standard peptide synthesis means known in the art, for example. Immunogens can be prepared commercially by one of numerous companies that do chemical synthesis. An example such a company is American Peptides, Inc., where the distributor is CLINALFA AG (Laufelfingen, Switzerland). The antigens or immunogens can be prepared in accordance with GMP standards and purity can be assessed by analytical HPLC. The product can be characterized by amino-acid analysis and tested for sterility and the absence of pyrogens.

The immunogenic compositions can also include adjuvants or other biological response modifiers (BRMs). Particularly advantageous methods of using adjuvants and BRMs are disclosed in U.S. Provisional Patent Application 60/640,727, filed Dec. 29, 2004 and U.S. patent application Ser. No. ______ (Pub. No. ______) (Attorney Docket No. MANNK.046A), filed on even date with the instant application, both entitled, “METHODS TO TRIGGER, MAINTAIN AND MANIPULATE IMMUNE RESPONSES BY TARGETED ADMINISTRATION OF BIOLOGICAL RESPONSE MODIFIERS INTO LYMPHOID ORGANS,” each of which is hereby incorporated by reference in its entirety.

An antigen can be delivered to an animal's system either directly or indirectly. For example, a polypeptide can be delivered directly as the polypeptide, or it can be delivered indirectly, for example, using a DNA construct or vector, or a recombinant virus that codes for the desired antigen. Any vector driving expression in a professional antigen presenting cell can be suitable for this purpose. In indirect delivery, the antigen is expressed in the cell, then presented by the MHC Class I on the surface of the cell to stimulate a CTL response. Expression of a secreted form of the antigen can be useful to induce an antibody response recognizing antigens that are membrane proteins.

In a preferred embodiment, an encoded antigen can be delivered in the form of a naked plasmid expression vector. Particularly useful constructs are disclosed in U.S. patent application Ser. No. 09/561,572, filed Apr. 28, 2000, Ser. No. 10/225,568 (Pub No. 2003-0138808), filed Aug. 20, 2002; and PCT Application No. PCT/US2003/026231 (Pub. No. WO 2004/018666); all entitled “EXPRESSION VECTORS ENCODING EPITOPES OF TARGET-ASSOCIATED ANTIGENS;” U.S. patent application Ser. No. 10/292,413 (Pub. No. 2003-0228634 A1), filed Nov. 17, 2002, Ser. No. 10/777,053 (Pub. No. 2004-0132088 A1), filed Feb. 10, 2004, and Ser. No. 10/837,217 (Pub. No. ______), filed Apr. 30, 2004, all entitled “EXPRESSION VECTORS ENCODING EPITOPES OF TARGET-ASSOCIATED ANTIGENS AND METHODS FOR THEIR DESIGN;” U.S. Pat. No. 6,709,844, and U.S. patent application Ser. No. 10/437,830 (Pub. No. 2003-0180949 A1), filed May 13, 2003, both entitled “AVOIDANCE OF UNDESIRABLE REPLICATION INTERMEDIATES IN PLASMIND PROPAGATION,” and in U.S. patent application Ser. No. 10/026,066, filed Dec. 7, 2001 (Pub. No. 2003-0215425 A1), filed Dec. 7, 2001, Ser. No. 10/895,523 (Pub. No. 2005-0130920 A1), filed Jul. 20, 2004, Ser. No. 10/896,325 (Pub. No. ______), filed Jul. 20, 2004, all entitled “EPITOPE SYNCHRONIZATION IN ANTIGEN PRESENTING CELLS,” each of which is hereby incorporated by reference in its entirety. Additional methodology, compositions, peptides, and peptide analogues are disclosed in U.S. Provisional Application No. 60/581,001, filed Jun. 17, 2004, and U.S. patent application Ser. No. 11/156,253 (Pub. No. ______), filed on Jun. 17, 2005, both entitled “SSX-2 PEPTIDE ANALOGS;” U.S. Provisional Patent Application No. 60/580,962, filed Jun. 17, 2004, and U.S. patent application Ser. No. 11/155,929 (Pub. No. ______), filed on Jun. 17, 2005, both entitled “NY-ESO PEPTIDE ANALOGS;” U.S. patent application Ser. No. 09/999,186, filed Nov. 7, 2001, entitled “METHODS OF COMMERCIALIZING AN ANTIGEN”; U.S. Provisional Patent Application No. 60/640,402, filed on Dec. 29, 2004, and U.S. patent application Ser. No. ______ (Pub. No. ______) (Attorney Docket No. MANNK.047A), filed on even date with the instant application, both entitled, “METHODS TO ELICIT, ENHANCE AND SUSTAIN IMMUNE RESPONSES AGAINST MHC CLASS I-RESTRICTED EPITOPES, FOR PROPHYLACTIC OR THERAPEUTIC PURPOSES”; and U.S. Provisional Patent Application No. 60/640,821, filed on Dec. 29, 2004, and U.S. patent application Ser. No. ______ (Pub. No. ______) (Attorney Docket No. MANNK.048A), filed on even date with the instant application both entitled “METHODS TO BYPASS CD4+ CELLS IN THE INDUCTION OF AN IMMUNE RESPONSE,” and U.S. Provisional Patent Application Nos. 60/691,579, filed Jun. 17, 2005, entitled “METHODS AND COMPOSITIONS TO ELICIT MULTIVALENT IMMUNE RESPONSES AGAINST DOMINANT AND SUBDOMINANT EPITOPES EXPRESSED ON CANCER CELLS AND TUMOR STROMA,” and 60/691,581, filed Jun. 17, 2005, entitled “MULTIVALENT ENTRAIN-AND-AMPLIFY IMMUNOTHERAPEUTICS FOR CARCINOMA,” each of which is hereby incorporated by reference in its entirety. The feasibility of and general procedures related to the use of naked DNA for immunization are described in U.S. Pat. No. 5,589,466, entitled “INDUCTION OF A PROTECTIVE IMMUNE RESPONSE IN A MAMMAL BY INJECTING A DNA SEQUENCE” and in U.S. Pat. No. 5,679,647, entitled “METHODS AND DEVICES FOR IMMUNIZING A HOST AGAINST TUMOR-ASSOCIATED ANTIGENS THROUGH ADMINISTRATIONS OF NAKED POLYNUCLEOTIDES WHICH ENCODE TUMOR-ASSOCIATED ANTIGENIC PEPTIDES,” each of which is hereby incorporated by reference in its entirety. The former teaches only intramuscular or intradermal injection while the latter teaches only administration to skin or mucosa.

In a preferred embodiment, the antigen can be administered directly to the lymphatic system. Intranodal administration for the generation of CTL is taught in U.S. patent application Ser. No. 09/380,534, filed Sep. 1, 1999 and U.S. Pat. No. 6,977,074, and in PCT Application No. PCTUS98/14289 (Pub. No. WO 99/02183 A2), filed on Jul. 10, 1998 each entitled “METHOD OF INDUCING A CTL RESPONSE,” each of which is hereby incorporated by reference in its entirety. Single bolus injection intra lymph node (i.ln.) required only 0.1% of the dose required in order to obtain a similar level of CTL response by intramuscular (i.m.) injection. Therefore a protective response can be established against systemic viral infection with a single bolus delivered i.ln., but not with a dose nearing the practical limit delivered i.m. Repeated bolus injections i.m. failed to establish a protective response against a peripheral virus infection or transplanted tumor, whereas lower doses administered i.ln. were completely effective. Particularly useful intranodal immunization protocols are taught in U.S. Provisional Patent Application No. 60/479,393, filed Jun. 17, 2003, entitled “METHODS TO CONTROL MHC CLASS I-RESTRICTED IMMUNE RESPONSE,” and in U.S. patent application Ser. No. 10/871,707 (Pub. No. 2005-0079152 A1), filed on Jun. 17, 2004, and U.S. Provisional Patent Application No. 60/640,402, filed on Dec. 29, 2004, both entitled “METHODS TO ELICIT, ENHANCE AND SUSTAIN IMMUNE RESPONSES AGAINST MHC CLASS I-RESTRICTED EPITOPES, FOR PROPHYLACTIC OR THERAPEUTIC PURPOSE,” each of which is hereby incorporated by reference in its entirety.

A class of epitopes that can be advantageous in anti-cancer immunogenic compositions are housekeeping epitopes. These are produced through the action of the housekeeping (or standard) proteasome. Housekeeping epitopes can be liberated from the translation product of expression vectors through proteolytic processing by the immunoproteasome of professional antigen presenting cells (pAPC). In one embodiment of the invention, sequences flanking the housekeeping epitope(s) can be altered to promote cleavage by the immunoproteasome at the desired location(s). Housekeeping epitopes, their uses, and identification are described in U.S. patent application Ser. No. 09/560,465 filed on Apr. 28, 2000, Ser. No. 10/026,066 (Pub. No. 2003-0215425 A1), filed on Dec. 7, 2001, Ser. No. 10/895,523 (Pub No. 2005-0130920 A1), filed Jul. 20, 2004, Ser. No. 10/896,325 (Pub. No. ______), filed Jul. 20, 2004, each entitled “EPITOPE SYNCHRONIZATION IN ANTIGEN PRESENTING CELLS,” and U.S. Pat. No. 6,861,234, and U.S. patent application Ser. No. 10/956,401 (Pub. No. 2005-0069982 A1), filed Oct. 1, 2004, both entitled “METHOD OF EPITOPE DISCOVERY,” each of which is hereby incorporated by reference in its entirety.

Examples of housekeeping epitopes are disclosed in U.S. Provisional Patent Applications Nos. 60/282,211, filed on Apr. 6, 2001; 60/337,017, filed on Nov. 7, 2001; 60/363,210, filed Mar. 7, 2002; and 60/409,123, filed on Sep. 6, 2002; U.S. patent application Ser. No. 10/117,937 (Publication No. 2003-0220239 A1), filed on Apr. 4, 2002; Ser. No. 10/657,022, filed on Sep. 5, 2003 (Pub. No. 2004-0180354 A1, and PCT Application No. PCT/US2003/027706, filed Sep. 5, 2003 (Pub. No. WO 04/022709 A2), all entitled “EPITOPE SEQUENCES,” each of which is hereby incorporated by reference in its entirety.

In other embodiments of the invention, the housekeeping epitope(s) can be flanked by arbitrary sequences or by sequences incorporating residues known to be favored in immunoproteasome cleavage sites. As used herein the term “arbitrary sequences” refers to sequences chosen without reference to the native sequence context of the epitope, their ability to promote processing, or immunological function. In further embodiments of the invention multiple epitopes can be arrayed head-to-tail. These arrays can be made up entirely of housekeeping epitopes. Likewise, the arrays can include alternating housekeeping and immune epitopes. Alternatively, the arrays can include housekeeping epitopes flanked by immune epitopes, whether complete or distally truncated. Further, the arrays can be of any other similar arrangement. There is no restriction on placing a housekeeping epitope at the terminal positions of the array. The vectors can additionally contain authentic protein coding sequences or segments thereof containing epitope clusters as a source of immune epitopes. The term “authentic” refers to natural protein sequences.

Epitope clusters and their uses are described in U.S. patent application Ser. No. 09/561,571, entitled “EPITOPE CLUSTERS,” filed on Apr. 28, 2000; Ser. No. 09/560,465, filed Apr. 28, 2000, Ser. No. 10/005,905, filed on Nov. 7, 2001, Ser. No. 10/026,066 (Pub. No. 2003-0215424 A1), filed on Dec. 7, 2001, Ser. No. 10/895,523 (Pub. No. 2005-0130920 A1), filed Jul. 20, 2004, Ser. No. 10/896,325 (Pub. No. ______), filed Jul. 20, 2004, each entitled “EPITOPE SYNCHRONIZATION IN ANTIGEN PRESENTING CELLS,” and Ser. No. 10/094,699, filed Mar. 7, 2002, and Ser. No. 11/073,347 (Pub. No. ______), filed on Jun. 30, 2005, each entitled ANTI-NEOVASCULATURE PREPARATIONS FOR CANCER, each of which is hereby incorporated by reference in its entirety.

In another embodiment of the invention an encoded antigen can be delivered in the form of a viral vector. A wide array of viruses with modified genomes adapted to express interposed reading frames but often no, or at least a reduced number of, viral proteins are known in the art, including without limitation, retroviruses including lentiviruses, adenoviruses, parvoviruses including adeno-associated virus, herpesviruses, and poxviruses including vaccinia virus. Such viral vectors facilitate delivery of the nucleic acid component into the cell allowing for expression. A subset of these vectors, such as retroviruses and parvoviruses, promote integration of their nucleic acid component into the host genome, whereas others do not.

Bacteria can also serve as vectors, that is they can be used to deliver a nucleic acid molecule capable of causing expression of an antigen. For example, a strain of Listeria monocytogenes has been devised that effects its own lysis upon entering the cytosol of macrophages (its normal target), thereby releasing plasmid from which antigen is subsequently expressed (Dietrich, G. et al., Biotechnology 16:181-185, 1998, which is hereby incorporated by reference in its entirety). Shigela flexneri and Escherichia coli have been similarly used (Sizemore, D. R. et al., Science 270:299-302, 1995, and Courvalin, P. et al., Life Sci. 318:1207-1212, 1995, respectively, each of which is hereby incorporated by reference in its entirety).

The use of microbial vectors for nucleic acid delivery can be complicated by the immune reactions the vectors themselves provoke. When prolonged or repeated administration is required, antibody elicited by the earlier treatment can prevent useful quantities of the vector from ever reaching its intended host. However, by direct administration intra lymph node, for example, the combination of proximity to host cells and the much reduced effective dose makes it possible to administer a dose capable of evading or overwhelming an existing antibody titer.

The word vector has been used, here and elsewhere, in reference to several modalities and variously modified (e.g., expression vector, viral vector, delivery vector, etc.). The underlying principle is that a nucleic acid capable of causing expression of an antigen, rather than the antigen itself, ultimately arrives in an APC. Unless modified, explicitly or by local context, in preferred embodiments, the term vector as used herein is intended to encompass all such possibilities.

The techniques discussed above are distinct from the approach of modifying the microbial genome, including extra-chromosomal DNA, such that the antigen is produced as a component of the microbe, which is then itself administered as the immunogen. Examples of microbes used in the genomic modification approach include viruses, bacteria, fungi, and protozoa. In embodiments of the invention described herein, the compositions, including the vaccines, can include the already synthesized antigen or a nucleic acid capable of causing an APC to express the antigen in vivo. In alternative embodiments, combinations of these two techniques are used. For example, one embodiment contemplates the use of a virus vector as discussed above that also incorporates a target epitope into a capsid or envelope protein.

Antigens may be used alone or may be delivered in combination with other antigens or with other compounds such as cytokines. Cytokines that are known to enhance immune stimulation of CTL responses, include, for example, GM-CSF, IL-12, IL-2, TNF, IFN, IL-18, IL-3, IL-4, IL-8, IL-9, IL-13, IL-10, IL-14, IL-15, G-SCF, IFN alpha, IFN beta, IFN gamma, TGF alpha, TGF beta, and the like. Cytokines are known in the art and are readily available in the literature or commercially. Many animal and human tumors have been shown to produce cytokines, such as IL-4, IL-10, TGF-B, that are potent modulators of the immune response and that protect tumors from immune-mediated destruction. The production of IL-4, IL-10 or TGF-B by tumors may achieve this protective effect by suppressing the induction of cellular immunity, including the elaboration of CTL responses. Alternatively, cytokines that support CTL responses can be exogenously added to help in the balance between induction of anti-tumor cell mediated and non-tumor-destructive humoral responses. Several such exogenous cytokines show utility in experimental mouse vaccination models which are known to enhance CTL responses, including GM-CSF, IFN and IL-2. An example of an effective exogenous cytokine that can be used is GM-CSF. GM-CSF is reported to enhance the expression of the so called “co-stimulatory” molecules, such as B7-1 or B7-2 on antigen presenting cells (APC). These co-stimulatory molecules are important players in the variety of interactions that occur during stimulation of CTL by APC. Moreover, GM-CSF is known to induce activation of APCs and to facilitate growth and differentiation of APCs, thereby making these APCs important CTL stimulating cells available both in greater numbers and potency.

Immunogenic compositions can additionally contain non-target antigens in order to improve the response to the target antigen. Thus, co-induction of a helper response, such as Th and/or B cell immunity against non-self or foreign antigens not expressed within the tumoral process or in the body, can result in a substantial improvement in the magnitude and quality of the immune response to the “self” or “self-modified” target antigens expressed within the tumor or underlying stroma. For example, co-initiating a Th immune response against a non-target antigen such as tetanus toxoid can result in the generation of helper cells with bystander effect relative to generation of CTL or B cell responses against the target tumor or self antigens. Any defined sequence expressing or encompassing peptide motifs that bind to at least one class II MHC protein expressed by recipient, where such sequences are non-homologous or contain non-homologous segments relative to self antigens, can be used. Preferably, such sequences are of microbial origin and shown to be immunogenic in HLA-defined or broader populations. In addition to tetanus toxoid (whole or portions, including, but not limited to, portions that are 90%, 80%, 75%, 70%, 60%, 50%, 40%, 30%, 25%, 20%, 15%, 10%, or 5% of a whole toxoid), further examples include, but are not limited to, sequences derived from HBVcore, influenza hemagglutinin, Plasmodium circumsporozoite antigen, and HTLV-1 envelope protein, and fragments of these sequences that are 90%, 80%, 75%, 70%, 60%, 50%, 40%, 30%, 25%, 20%, 15%, 10%, or 5% of the respective full-length sequences. In some embodiments, the tetanus toxoid portion is 5% to 90% of a whole toxoid, in other embodiments, the portion is 15% to 80% of a whole toxoid, in still other embodiments, the toxoid portion is 25% to 70% of a whole toxoid, in yet other embodiments, the toxoid portion is 35% to 60% of a whole toxoid, in still other embodiments, the toxoid portion is 45% to 55% of a whole toxoid. Similarly, co-administration of a strongly immunogenic B cell epitope (a non-self antigen) with or without a Th epitope (a non-self antigen) with target epitopes (self, tumoral) in a cognate fashion (that is, within the same molecule), can result in improved immune response, or even break of tolerance (T cell) against the therapeutic target, via immune antibody-antigen complexes and bystander T cell help.

Delivery of the Antigen

While not wanting to be bound by any particular theory, it is thought that T cells do not have a functional memory that is long-lived. Antibody-mediated B-cell memory, on the other hand, appears to have a long-lived effector memory. Thus, delivering an antigen that induces a CTL response is most preferably done over time to keep the patient's immune system appropriately stimulated to attack the target cells. In one approach the presence of antigen is maintained virtually continuously within the lymphatic system to maintain effector CTL function as disclosed in U.S. Pat. No. 6,977,074, entitled “METHOD OF INDUCING A CTL RESPONSE,” which is hereby expressly incorporated by reference. In another approach T cell memory is repeatedly induced, and re-amplified and reactivated as described in U.S. Provisional Patent Application No. 60/479,393, entitled “METHODS TO CONTROL THE MHC CLASS I-RESTRICTED IMMUNE RESPONSE,” filed Jun. 17, 2003, and in U.S. patent application Ser. No. 10/871,707, entitled “METHODS TO ELICIT, ENHANCE AND SUSTAIN IMMUNE RESPONSES AGAINST MHC CLASS I-RESTRICTED EPITOPES, FOR PROPHYLACTIC OR THERAPEUTIC PURPOSE” (Pub. No. 2005-0079152 A1), filed Jun. 17, 2004, each of which is hereby incorporated by reference in its entirety. While it has been suggested that antigens and adjuvants can be prepared as biodegradable microspheres or liposomes, none of these preparations have thus far provided a CTL response that is useful for attacking cancer cells or pathogens on a long term basis. Preferably, delivery of the antigen is sustained over the desired period of time at a level sufficient to maintain the antigen level to obtain the desired response. In one embodiment, a reservoir having fluid antigen composition can be used to deliver the antigen such that it reaches the animal's lymphatic system. While much of the following discussion focuses on the use of infusion to deliver the antigen it is also possible to use bolus injections directly into the lymphatic system, the number and frequency of which will depend on the persistence of antigen conferred by the particular form and formulation of antigen used.

Ultimately antigen finds its way into the lymphatic system in order to most efficiently stimulate CTL. Delivery of antigen can involve infusion into various compartments of the body, including but not limited to subcutaneous, intravenous, intraperitoneal and intralymphatic, the latter being preferred. While each of these points of infusion results in antigen uptake into the lymphatic system, the relative amounts of antigen needed to induce a beneficial CTL response varies according to the site of infusion. In general, direct infusion of antigen into the lymph system is deemed to be the most efficient means of inducing a CTL response, however, any delivery route can be used. Pump systems are capable of delivering material quantities of antigen in a range that is suitable for inducing a CTL response through delivery to all compartments of the body. CTL stimulation following delivery of antigen via the various routes will vary depending on the properties of different antigens, including factors that influence antigen behavior in the body and its rate of equilibration to (or longevity in) the lymph, such as antigen stability in the body fluid, solubility of antigen in body fluid, binding affinity for HLA and potency as a stimulator of CTL.

In a preferred embodiment, introduction of the antigen is done as directly as possible to the lymphatic system to avoid the destruction of the antigen by metabolism in the body. When introduction of a fluid antigen composition occurs subcutaneously, larger quantities of antigen are needed to assure enough antigen reaches the lymphatic system. Such subcutaneous injection is contemplated by the invention disclosed herein, depending on factors such as cost, stability of the antigen, how quickly the antigen gets to the lymph system, how well it equilibrates with the lymph, and other factors that the attending doctor or specialist will recognize. Subcutaneous delivery generally can require 100 to 1000 times more antigen than direct delivery to the lymph system. It is preferable, therefore, that the antigen composition is introduced through a device for local administration to the lymphatic system, e.g., the spleen, a lymph node, or a lymph vessel. The device for local administration can be positioned outside the patient or implanted into the patient. In either case, the device can have a reservoir to hold the fluid antigen-containing composition, a pump to transfer the composition, and a transmission channel leading from the reservoir to be directed to the preferred region of administration in the patient's body. In either case it is preferably portable.

For the device positioned outside the patient's body (the external device), there are numerous devices used for delivering insulin to diabetic patients that are useful in delivering antigen according to the embodiments described herein. Generally these devices can be comprised of a reservoir for holding the antigen composition (instead of insulin), a programmable pump to pump the composition out of the reservoir, a transmission channel or line for transmitting the composition, and a means to introduce the composition into the animal's body to ultimately reach the lymphatic system.

Preferably, the reservoir for the antigen composition should be large enough for delivery of the desired amount of antigen over time and easily refillable or replaceable without requiring the user to reinsert the means for introducing the antigen composition to the lymph system.

In preparing the antigen compositions of embodiments of the invention disclosed herein, a composition (preferably aqueous) can be prepared to be compatible with the lymph system and physiologically acceptable to the animal being treated. Relevant considerations include, for example, the physicochemical properties of the antigen, such as the isoelectric point, molecular weight, glycosylation or other post-translational modification, and overall amino acid composition. These properties along with any known behavior of the drug in different solutions (e.g., different buffers, cofactors, etc.) as well as its in vivo behavior can help guide the choice of formulation components. One parameter that impacts all the major degradation pathways is the solution pH. Thus, the initial formulations also assess the pH dependence of the degradation reactions and the mechanism for degradation, which can often be determined from the pH dependence to determine the stability of the protein in each solution. Rapid screening methods usually involve the use of accelerated stability at elevated temperatures (e.g., 40° C.) using techniques known in the art.

In general the antigen compositions useful in embodiments described herein can be suitable for parenteral injection, in very small quantities. As such a composition should be free of contamination and have a pH compatible with the lymphatic system. However, because very small quantities of the antigenic composition will be delivered it need not be the same pH as blood or lymph, and it need not be aqueous-based. The preferable pH range that is compatible is from about 6.7-7.3 and can be prepared using water for injection to meet USP specifications (see Remington: The Science and Practice of Pharmacy, Nineteenth Edition; Chapters 86-88, which is hereby incorporated by reference in its entirety). For antigens that are less soluble, a suitable cosolvent or surfactant can be used, such as dimethyl sulfoxide (DMSO) or PLURONIC brand surfactants. Generally, a standard saline solution that is buffered with a physiologically acceptable weak acid and its base conjugate, e.g., a phosphate or citrate buffering system, will be the basis of the antigen composition. In some cases, a small amount of an antioxidant may be useful to stabilize the composition and prevent oxidation. Factors to consider in preparing the antigen compositions can be found in the 1994 American Chemical Society book entitled “Formulation and Delivery of Proteins and Peptides” (Acs Symposium Series, No. 567) by Jeffery L. Cleland and Robert Langer (Editor), which is hereby incorporated by reference in its entirety.

For nucleic acid encoded antigens similar considerations can apply, although the variety of physico-chemical properties encountered with polypeptides is absent, so that acceptable formulations will have nearly universal applicability. As seen in Examples 6-10, plasmid DNA in standard phosphate buffered saline (PBS) is an acceptable and effective formulation. In some embodiments of the invention, DNA is administered continuously or intermittently at short intervals, from a reservoir worn on, or implanted in, the patient's body. It is preferable that the DNA be maintained in a soluble, stable form at or near body temperature over a period of time measured minimally in days. In such applications where the formulated nucleic acid will be delivered from a reservoir over a period of several days or longer, the stability of the nucleic acid at room or body temperature for that period of time, as well as its continued sterility, take on increased importance. The addition of bacteriostatic agents (e.g., benzyl or ethyl alcohol) and chelating agents (e.g., EDTA) is useful toward these ends. Formulations containing about 0.5-2% ethyl alcohol, 0.25-0.5 mM EDTA generally perform well. Such formulations are also appropriate for bolus injections.

Generally the amount of the antigen in the antigen composition will vary from patient to patient and from antigen to antigen, depending on such factors as the activity of the antigen in inducing a response and the flow rate of the lymph through the patient's system. In general the antigen composition may be delivered at a rate of from about 1 to about 500 microliters/hour or about 24 to about 12000 microliters/day. The concentration of the antigen is such that about 0.1 micrograms to about 10,000 micrograms of the antigen will be delivered during 24 hours. The flow rate is based on the knowledge that each minute approximately about 100 to about 1000 microliters of lymph fluid flows through an adult inguinal lymph node. The objective is to maximize local concentration of vaccine formulation in the lymph system. A certain amount of empirical investigation on patients will be necessary to determine the most efficacious level of infusion for a given vaccine preparation in humans.

To introduce the antigen composition into the lymphatic system of the patient the composition is preferably directed to a lymph vessel, lymph node, the spleen, or other appropriate portion of the lymphatic system. Preferably, the composition is directed to a lymph node such as an inguinal or axillary node by inserting a catheter or needle to the node and maintaining the catheter or needle throughout the delivery. Suitable needles or catheters are available made of metal or plastic (e.g., polyurethane, polyvinyl chloride (PVC), TEFLON, polyethylene, and the like). In inserting the catheter or needle into the inguinal node for example, the inguinal node is punctured under ultrasonographic control using a Vialon™ Insyte-W™ cannula and catheter of 24G3/4 (Becton Dickinson, USA) which is fixed using Tegaderm™ transparent dressing (Tegaderm™ 1624, 3M, St. Paul, Minn. 55144, USA). This procedure is generally done by an experienced radiologist. The location of the catheter tip inside the inguinal lymph node is confirmed by injection of a minimal volume of saline, which immediately and visibly increases the size of the lymph node. The latter procedure allows confirmation that the tip is inside the node. This procedure can be performed to ensure that the tip does not slip out of the lymph node and can be repeated on various days after implantation of the catheter. In the event that the tip does slip out of location inside the lymph node, a new catheter can be implanted.

Formulation and Treatment Protocol

There are several approaches to utilizing the combination of TuAAs with DNA vaccines. A first approach is to include all the antigens or epitopes from all the antigens in a given combination into a single DNA expression vector. This approach has the advantages of simplicity for manufacturing and administration to patients. However, in some instances, epitope competition can limit the usefulness of this approach. That is, it is possible that only the most immunogenic epitope will elicit an immune response when a vaccine with several epitopes representing all TuAAs in the combination is given to patients. It is also more difficult to design and construct a DNA vaccine in which all epitopes are expressed at high efficiencies. Nevertheless, because the procedure for treating patients is simple and uniform within each type of cancer, the cost is likely to be lower than for the other approaches described below.

An alternate approach is to include only one antigen or epitopes of one antigen in a DNA expression vector. This approach has the advantages of simplicity in designing and constructing the DNA vector, flexibility, and customized administration to patients. If a large number of individual TuAA vaccines are available, then one can customize treatment for each individual patient based on the TuAA expression profile of his or her tumor. For example, if the standard combination for treating a given type of cancer is TuAAs A, B, and C (where A, B, and C designate different tumor associated antigens), but a patient's tumor expresses TuAAs A, C, and Z (but not B), then the patient can be treated with separate vaccines for each of A, C, and Z. This flexibility and customizability improves the success rate of immunotherapy because antigen redundancy can be achieved for each patient. However, the procedure of treating the patient can be more complex. For example, delivery using this approach can include a sequential administration scheme (one antigen at a time), or injection into multiple, anatomically separate sites of the patient at about the same time.

Still another approach is to combine epitopes from multiple TuAAs that have similar immunogenicity into a DNA expression vector (more than one vector may be used for some combinations).

A profile of the antigen expression of a particular tumor can be used to determine which antigen or combination of antigens to use. Exemplary methodology and specific antigenic combinations of particular benefit in directing an immune response against particular cancers are disclosed in U.S. Provisional Patent Application No. 60/479,554, filed on Jun. 17, 2003, U.S. patent application Ser. No. 10/871,708 (Publication No. 2005-0118186 A1), filed on Jun. 17, 2004, and PCT Patent Application No. PCT/US2004/019571 (Pub. No. WO 2004/112825), filed Jun. 17, 2004, U.S. Provisional Patent Application No. 60/640,598, filed Dec. 29, 2004, and U.S. patent application Ser. No. ______ (Publication No. ______) (Attorney Docket number MANNK.049A) filed on the same date as the instant application, all entitled “COMBINATIONS OF TUMOR-ASSOCIATED ANTIGENS IN COMPOSITIONS FOR VARIOUS TYPES OF CANCER”, each of which is also hereby incorporated by reference in its entirety.

Patients that can benefit from such methods of immunization can be recruited using methods to define their MHC protein expression profile and general level of immune responsiveness. In addition, their level of immunity can be monitored using standard techniques in conjunction with access to peripheral blood. Finally, treatment protocols can be adjusted based on the responsiveness to induction or amplification phases and variation in antigen expression. For example, rather than amplifying after some set number of entrainment doses, repeated entrainment doses can be administered until a detectable response is obtained, and then amplifying peptide dose(s) can be administered. Similarly, scheduled amplifying or maintenance doses of peptide can be discontinued if their effectiveness wanes, antigen-specific regulatory T cell numbers rise, or some other evidence of tolerization is observed, and further entrainment can be administered before resuming amplification with the peptide. The integration of diagnostic techniques to assess and monitor immune responsiveness with methods of immunization is discussed more fully in U.S. Provisional Patent Application No. 60/580,964, filed on Jun. 17, 2004, and U.S. patent application Ser. No. 11/155,928 (Pub. No. ______), filed on Jun. 17, 2005, both entitled “IMPROVED EFFICACY OF ACTIVE IMMUNOTHERAPY BY INTEGRATING DIAGNOSTIC WITH THERAPEUTIC METHODS,” each of which is hereby incorporated by reference in its entirety.

Combination of active immunotherapies, as disclosed herein, with other treatment modalities can increase the susceptibility of tumoral processes to the elicited immune response and thereby result in increased therapeutic benefit. In some embodiments, the therapeutic benefit is synergistically enhanced. Tumor debulking prior to or during active immunotherapy increases the potential for any particular level of immune response to slow or halt disease progression or to bring about tumor regression or elimination. Additionally, tissue damage, necrosis, or apoptosis initiated with antibody therapy, radiotherapy, biotherapy, chemotherapy, passive immunotherapy (including treatment with mono- and/or polyclonal antibodies, recombinant TCR, and/or adoptive transfer of CTL or other cells of the immune system) or surgery, can facilitate the active immunotherapeutic approach via general inflammation resulting in recruitment of immune effector cells including antigen-specific effectors. In general, any method to induce a transient or more permanent general inflammation within one or multiple tumors/metastatic lesions can facilitate the active immunotherapy. Alternatively or in addition to enabling recruitment of effectors, general inflammation can also increase the susceptibility of target cells to immune mediated attack (e.g., as interferons increase expression of target molecules on cancer cells and underlying stroma). Still other strategies to increase susceptibility of tumor cells to immune mediated attack—by providing factors that interfere with the “stress response” or increase target molecules on cancer cells or stromal cells—can synergize with active immunotherapy.

In some embodiments, the numbers expressing quantities of ingredients, properties such as molecular weight, reaction conditions, and so forth used to describe and claim certain embodiments of the invention are to be understood as being modified in some instances by the term “about.” Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

In some embodiments, the terms “a” and “an” and “the” and similar referents used in the context of describing a particular embodiment of the invention (especially in the context of certain of the following claims) may be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Variations on those preferred embodiments will become apparent to those of ordinary skill in the art upon reading the foregoing description. It is contemplated that skilled artisans may employ such variations as appropriate, and the invention may be practiced otherwise than specifically described herein. Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that may be employed may be within the scope of the invention. Thus, by way of example, but not of limitation, alternative configurations of the present invention may be utilized in accordance with the teachings herein. Accordingly, the present invention is not limited to that precisely as shown and described.

Many variations and alternative elements of the invention have been disclosed. Still further variations and alternate elements will be apparent to one of skill in the art. Among these variations, without limitation, are the specific number of antigens in a screening panel or targeted by a therapeutic product, the type of antigen, the type of cancer, and the particular antigen(s) specified. Various embodiments of the invention can specifically include or exclude any of these variations or elements.

Each of the references cited herein is hereby incorporated herein by reference in its entirety.

The following examples are for illustrative purposes only and are not intended to limit the scope of the embodiments in any way.

### EXAMPLES

### Example 1

### TuAA Analysis and Selection of Combinations

The presence of TuAAs was measured by Real-Time PCR (RT-PCR). Briefly, total RNA was isolated from tumor specimens by standard methods and cDNA was made with standard reverse transcription procedures. Complementary DNA (cDNA) was amplified with specially designed, gene specific, primers that anneal only to cDNA but not genomic DNA. TuAA expression patterns of 12 ovarian and 7 colorectal tumor specimens were analyzed by RT-PCR. The results are summarized in the Table 4 below.

TABLE 4

| | Total # | PRAME | NY-ESO-1 | SSX-2 | PSMA | MAGE1 | MAGE3 |
|---|---|---|---|---|---|---|---|
| Ovarian | 12 | 12 | 5 | 6 | 6 | 4 | 3 |
| Colorectal | 7 | 5 | 1 | 2 | 5 | 0 | 1 |

### Example 2

### Ovarian Cancer

In the case of ovarian cancer, all samples analyzed were positive for PRAME. Thus the inclusion of PRAME in the combination improves coverage of the cases with ovarian cancer.

In order to achieve antigen redundancy and improve coverage in a large population, combinations of other antigens in addition to PRAME were considered. SSX-2 as well as PSMA were present in 6 of the 12 cases individually, but the combination of SSX-2 and PSMA provided coverage in 9 of 12 cases. Although NY-ESO-1 and SSX-2 were only present in 5 and 6 of the 12 cases, respectively, either NYESO-1 or SSX-2 was detected in 7 of the 12 cases.

Thus, for assembling panels, the combination of PRAME, SSX-2, and PSMA or PRAME, NY-ESO-1, and SSX-2 provided preferable coverage and redundancy compared to the combination of PRAME and PSMA or the combination of PRAME and SSX-2. The combination of PRAME, SSX-2, and PSMA provided excellent coverage of cases and good antigen redundancy because the majority of ovarian tumor samples analyzed had at least two of the four TuAA in the combination present. The combination of PRAME, SSX-2, PSMA, and NY-ESO-1 provided more preferred antigen redundancy, and thus, lower possibility of tumor escape.

### Example 3

### Colorectal Cancer

In the case of colorectal cancer, PRAME and PSMA were each detected in 5 of the 7 samples analyzed. In 6 of the 7 cases, either PRAME or PSMA was detected. Although SSX-2 was only detected in 2 of 7 cases, both SSX-2-PRAME and SSX2-PSMA combinations increased coverage to 6 of 7. Similarly, although NYESO-1 was detected in only 1 of 7 cases, the combination of NY-ESO-1-PRAME as well as the NYESO-1-PSMA combination increased coverage to 6 of 7. The addition of SSX-2 or NYESO-1 to the PRAME and PSMA combination improved coverage to 7 of 7. Thus, for assembling panels, the combination of PRAME, PSMA, and NYESO-1, or the combination of PRAME, PSMA, and SSX-2 provided good coverage of cases and redundancy of antigens for a majority of patients. The combination of PRAME, PSMA, NY-ESO-1, and SSX-2 provided further redundancy.

### Example 4

### Pancreatic Cancer

Real-Time PCR (RT-PCR) was utilized to determine the presence of PRAME, SSX2, NY-ESO-1, and PSMA. Briefly, total RNA was isolated from 5 pancreatic tumor specimens by standard methods and cDNA was made with standard reverse transcription procedures. Complementary DNA (cDNA) was amplified with specially designed, gene specific, primers that anneal only to cDNA but not genomic DNA.

In the pancreatic cancer specimens, the presence of PRAME, NYESO-1, SSX-2, and PSMA was detected in 100%, 40%, 20%, and 100% of the specimens, respectively (see Table 5). Elsewhere, PSMA and over-expression of HER2-/neu were reported to be present in 100% and 21% of pancreatic tumors, respectively (Chang S S et al., Cancer Res 1999, 59:3192; Safran H et al., Am J Clin Oncol. 2001, 24:496, each of which is hereby incorporated by reference in its entirety). Although over-expression of HER2/neu may render the cancer tissue a preferred target, thus providing some specificity for immunotherapy, low level expression of HER2/neu in normal tissues remains a concern. Thus, for assembling panels of antigens, the combination of NYESO-1, SSX-2, plus PRAME or PSMA provides excellent coverage and some redundancy for pancreatic cancer. Inclusion of both PRAME and PSMA significantly improves redundancy.

TABLE 5

| TAA | PRAME | SSX2 | NY-ESO-1 | PSMA |
|---|---|---|---|---|
| Detection Freq. | 5/5 | 1/5 | 2/5 | 5/5 |
| % positive | 100 | 20 | 40 | 100 |

### Example 5

### Renal Cell Carcinoma

For renal cell carcinoma, SSX-2, PSMA and PRAME were detected with frequencies of 5, 100 and 40%, respectively (Sahin, U et al., Clin Cancer Res. 2000, 6:3916; Chang S S et al., Urology 2001, 57:801; Neumann E et al., Cancer Res. 1998, 58:4090, each of which is hereby incorporated by reference in its entirety). Thus, the combination of PSMA and PRAME provides excellent coverage and redundancy for renal cell carcinoma. Adding SSX-2 to the combination of PSMA and PRAME improves redundancy.

### Example 6

### Non-Small Cell Lung Cancer

For non-small cell lung cancer, the reported presence of NYESO-1, SSX-2, MAGE-3, BAGE, over-expression of Her2/neu, and PSMA was 21, 15, 60, 6, 16, and 100%, respectively (Scanlan M J et al., Cancer lett 2000, 150:155; Chang S S et al., Cancer Res 1999, 59:3192; Selvaggi G et al., Cancer 2002, 94:2669, each of which is hereby incorporated by reference in its entirety). Thus, the combination of NYESO-1, SSX-2, MAGE-3, and PSMA provides coverage and antigen redundancy for the immunotherapy of non-small cell lung cancer.

### Example 7

### Melanoma

For melanoma, Melan A, Tyrosinase, NYESO-1, and SSX-2 were reported to be present in 92, 92, 41, and 35% of tumor specimens, respectively (Fetsch P A, et al., Cancer 1999, 87:37; Fetsch P A, et al., Cancer 2000, 90:252; Schultz-Thater E et al, Br J Cancer 2000, 83:204; Sahin, U et al., Clin Cancer Res. 2000, 6:3916). Therefore, the combination of Melan A, Tyrosinase, NYESO-1, and SSX-2 provides excellent coverage and antigen redundancy for the immunotherapy of melanoma. Significant redundancy is achieved using tyrosinase and melan-A together, or by combining NY-ESO-1 and SSX-2 with either of tyrosinase or melan-A.

### Example 8

Further studies involving the foregoing tumor types confirmed the observed expression patterns and preferred panels of TuAA. A total of 34 ovarian, 44 colon, 18 renal, and 13 pancreatic tissue samples obtained from various vendors were analyzed for tumor-associated antigen expression using qRTPCR. The results of these assays demonstrated that PRAME and PSMA were expressed frequently (ranging from 68% to 100%) in all four types of tumors studied. NY-ESO-1 and SSX2 were expressed in 20% to 40% of ovarian and pancreatic tumors.

TABLE 6
Overall Expression Profiles for Tumor Associated Antigens
From RTPCR analysis of Primary Tumors and Metastases

| Tumor- | | | | |
|---|---|---|---|---|
| Associated | % Samples Expressing a Given Antigen | | | |
| Antigen | Ovarian^{a} | Renal^{b} | Pancreatic^{c} | Colorectal^{d} |
| SSX2 | 36 | 6 | 20 | 8 |
| NY-ESO-1 | 30 | 6 | 40 | 12 |
| PRAME | 97 | 83 | 80 | 76 |
| PSMA | 91 | 100 | 100 | 68 |
| MAGE-1 | 27 | 6 | 33 | 8 |
| MAGE-3 | 30 | 22 | 42 | 20 |
| SCP-1 | 30 | 11 | 0 | 0 |
| CEA | 30 | 0 | 58 | 92 |

^{a}33 samples (27 primary tumors and 6 metastases)
^{b}18 samples (18 primary tumors)
^{c}15 samples (14 primary tumors and 1 metastasis; PSMA on 10 samples)
^{d}25 samples (13 primary tumors and 12 metastases)

### Example 9

### Schedule of Immunization with Plasmids Expressing Epitopes from Two Antigens

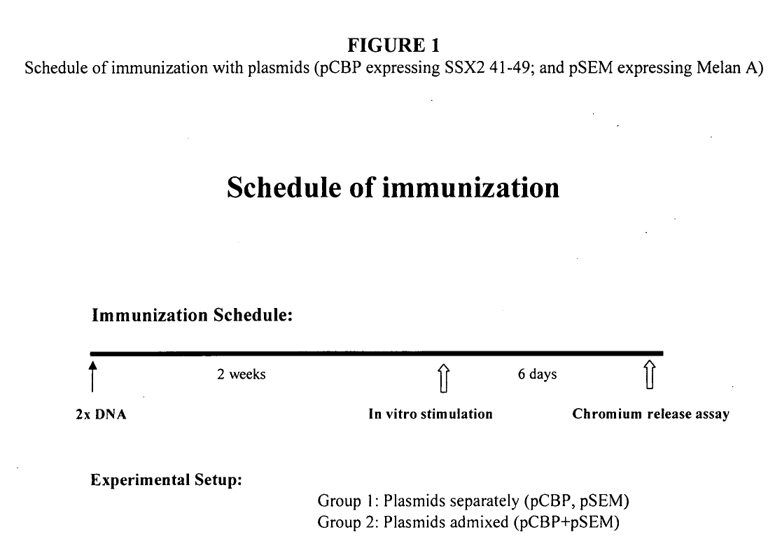

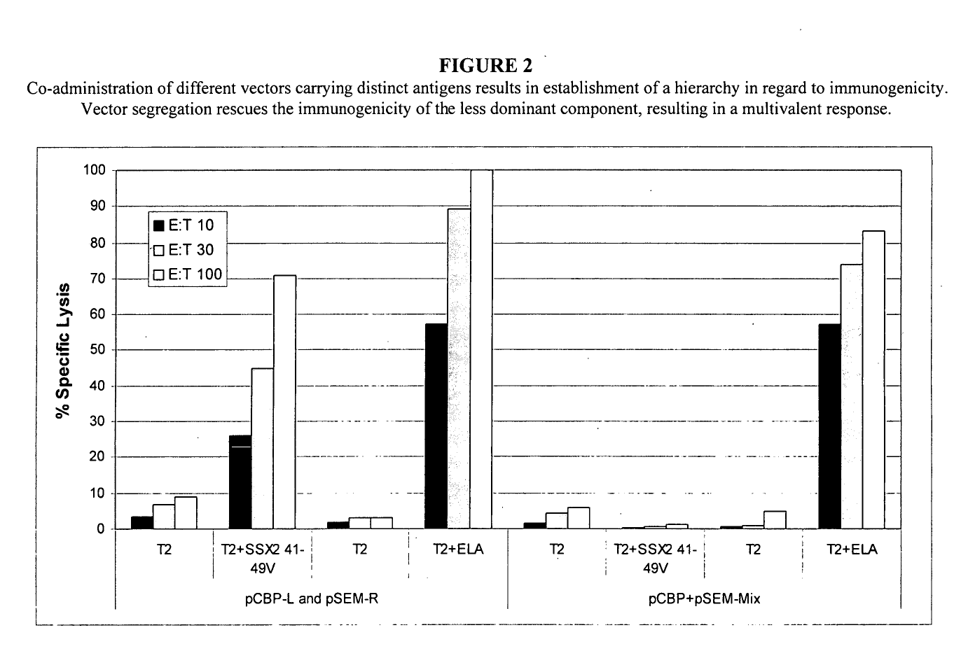

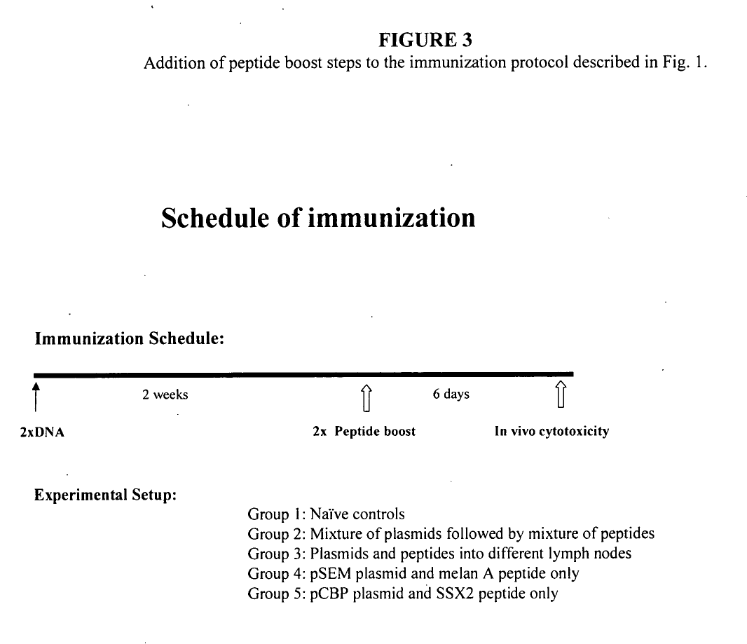

Two groups of HHD mice (n=4) were immunized via intra lymph node injection with either pSEM expressing Melan-A₂₆₋₃₅A27L (ELA) and pCBP expressing SSX-2₄₁₋₄₉ as a mixture; or with pSEM in the left inguinal lymph node and pCBP in the right inguinal lymph node, twice, at day 0 and 4 as shown in FIG. 1. The amount of the plasmid was 25 μg/plasmid/dose. Two weeks later, the animals were sacrificed, and cytotoxicity was measured against T2 cells pulsed or not with peptide.

### Example 10

### Co-Administration of Different Vectors Carrying Distinct Antigens

The animals immunized as described in Example 9, were sacrificed and splenocytes from each group pooled and stimulated with the two peptides (ELA or SSX-2₄₁₋₄₉) in parallel. The cytotoxicity was measured by incubation with Cr⁵¹-tagged, peptide loaded T2 target cells. Data in FIG. 2 show mean of specific cytotoxicity (n=4/group) against various target cells.

The results show that use of plasmid mixture interferes with the response elicited by pCBP plasmid; however, segregating the two plasmids relative to site of administration rescues the activity of pCBP. Thus, the co-administration of different vectors carrying distinct antigens can result in establishment of a hierarchy with regard to immunogenicity. Vector segregation can rescue the immunogenicity of the less dominant component, resulting in a multivalent response.

### Example 11

### Rescue of Multivalent Response by Addition of Peptide Boost Steps

Four groups of HHD mice (n=6) were immunized via intra lymph node injection with either pSEM and pCBP as a mixture; or with pSEM in the left inguinal lymph node and pCBP in the right inguinal lymph node, twice, at day 0 and 4 as shown in FIG. 3. As a control, mice were immunized with either pSEM or pCBP plasmid. The amount of the plasmid was 25 μg/plasmid/dose. Two weeks later, the animals were boosted with melan A and/or SSX-2 peptides, mirroring the plasmid immunization dose and combination. Two weeks later, the animals were challenged with splenocytes stained with CFSE and loaded or not with Melan A or SSX-2 peptide, for evaluation of in vivo cytotoxicity.

### Example 12

### Peptide Amplification Rescues the Immunogenicity of the Less Dominant Epitope

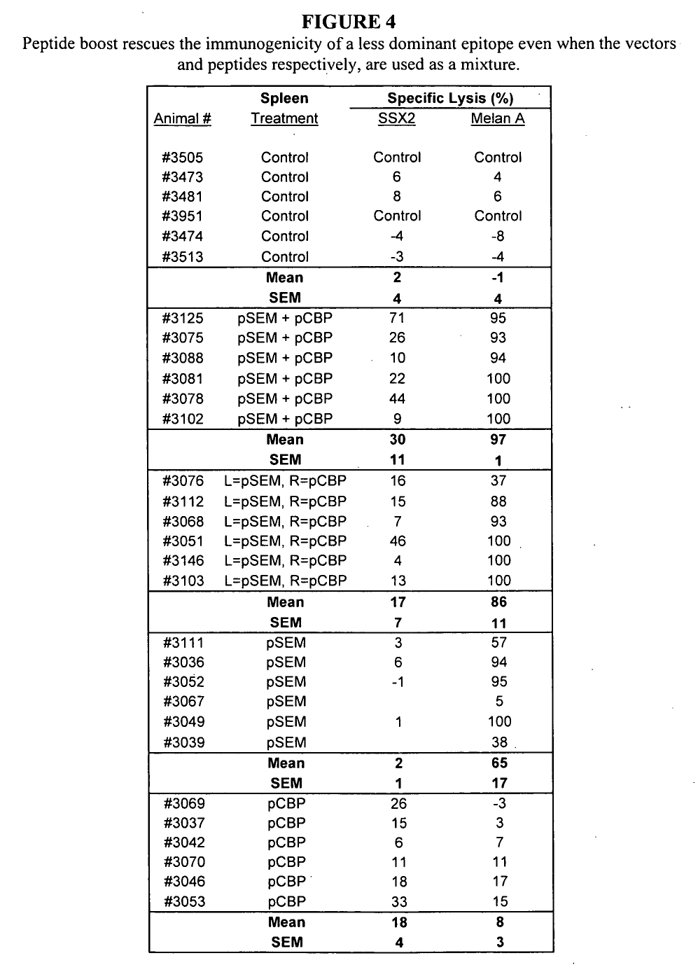

Mice were immunized as described in Example 11 and challenged with HHD littermate splenocytes coated with ELA or SSX-2 peptide, employing a triple peak CFSE in vivo cytotoxicity assay that allows the assessment of the specific lysis of two antigen targets simultaneously. Equal numbers of control-CFSE^{lo}, SSX-2₄₁₋₄₉-CFSE^{med}, and ELA-CFSE^{hi} cells were intravenously infused into immunized mice and 18 hours later the mice were sacrificed and target cell elimination was measured in the spleen (FIG. 4) by CFSE fluorescence using a flow cytometry. FIG. 4 shows the percent specific lysis of the SSX-2 and Melan-A antigen targets from individual mice, as well as the mean and SEM for each group.

The results show that immunizing the animals with a mixture of the two vaccines comprising plasmids followed by peptides generated immunity to both antigens and resulted in the highest immune response, representing an average SSX-2 percent specific lysis in the spleen of 30+/−11, and an average Melan-A percent specific lysis of 97+/−1.

### Example 13

### Clinical Practice for Entrain-and-Amplify Immunization

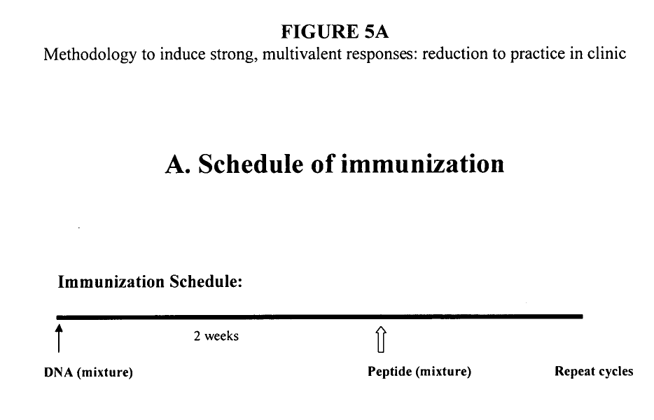

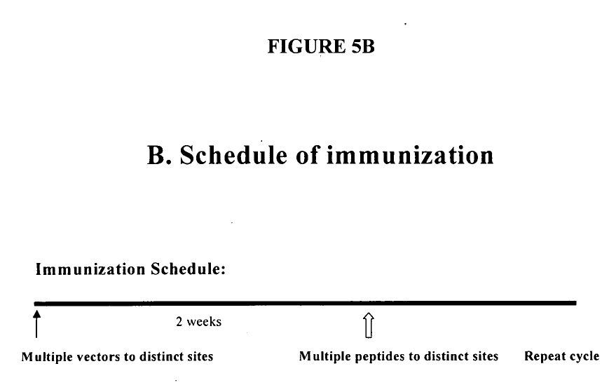

The data in FIGS. 2 and 4 suggest two scenarios for achieving a strong multivalent response in the clinic, shown in FIG. 5. In the first scenario (A), use of peptides for boosting restores multivalent immune responses even if plasmids and peptides are used as mixtures. In the second scenario (B), segregation of plasmid and peptide components respectively, allows induction of multivalent immune responses.

### Example 14

### MKC1207: an Entrain-and-Amplify Therapeutic for Melanoma

MKC1207 comprises the plasmid pSEM (described in U.S. patent application Ser. No. 10/292,413, (Pub. No. 2003-0228634 A1), filed Nov. 7, 2002, Ser. No. 10/777,053 (Pub. No. 2004-0132088 A1), filed Feb. 10, 2004, Ser. No. 10/837,217 (Pub. No. ______), filed Apr. 30, 2004, each of which is hereby incorporated by reference in its entirety, in which it is referred to as pMA2M) and peptides corresponding to Melan-A 26-35 and tyrosinase 369-377. The plasmid encodes the A27L analogue of the Melan-A epitope and the native tyrosinase epitope sequence. The plasmids encode both of these epitopes in such a manner that they can be expressed and presented by pAPC. In alternate embodiments of the therapeutic the peptides can comprise the native sequence or be analogues such as those disclosed in U.S. patent application Ser. No. 11/156,369 (Pub. No. ______), entitled EPITOPE ANALOGUES, filed on Jun. 17, 2005, and incorporated herein by reference in its entirety.

Briefly, the plasmid is administered intranodally to the inguinal lymph nodes as an entraining immunogen. Subsequently the peptides are administered intranodally, one to the left node, the other to the right as amplifying immunogens. The entrain-and-amplify protocol is described in greater detail in U.S. patent application Ser. No. 10/871,707 (Pub. No. 2005-0079152 A1), filed on Jun. 17, 2004 and 60/479,393, filed on Jun. 17, 2003, each of which is hereby incorporated by reference in its entirety.

Melanoma patients can be screened according to the methods disclosed herein and MKC1207 administered to patients whose tumor antigen profile includes Melan-A and/or tyrosinase. In a preferred embodiment the patient's tumor tissue also expresses HLA-A2, particularly HLA-A*0201.

### Example 15

### MKC1106: a Tetravalent Entrain-and-Amplify Therapeutic for Carcinoma

MKC1106 comprises the plasmids pCBP (described in U.S. patent application Ser. No. 10/292,413, (Pub. No. 2003/0228634 A1), filed Nov. 7, 2002, Ser. No. 10/777,053 (Pub. No. 2004-0132088 A1), filed Feb. 10, 2004, Ser. No. 10/837,217 (Pub. No. ______), filed Apr. 30, 2004, each of which is hereby incorporated by reference in its entirety) and pRP12 (described in U.S. Provisional Application No. 60/691,579, entitled METHODS AND COMPOSITIONS TO ELICIT MULTIVALENT IMMUNE RESPONSES AGAINST DOMINANT AND SUBDOMINANT EPITOPES, EXPRESSED ON CANCER CELLS AND TUMOR STROMA, filed on Jun. 17, 2005, and incorporated herein by reference in its entirety; and peptides corresponding to NY-ESO-1 157-165, SSX-2 41-49, PRAME 425-433 and PSMA 288-297. The plasmids encode both of these epitopes in such a manner that they can be expressed and presented by pAPC. In alternate embodiments of the therapeutic the peptides can comprise the native sequence or be analogues such as those disclosed in U.S. patent application Ser. No. 11/156,253 (Pub. No. ______), entitled SSX-2 PEPTIDE ANALOGS, and Ser. No. 11/155,929 (Pub. No. ______), entitled NY-ESO-1 PEPTIDE ANALOGS, and Ser. No. 11/156,369 (Pub. No. ______), entitled EPITOPE ANALOGS, and U.S. Provisional Patent Application No. 60/691,889, entitled EPITOPE ANALOGS, each of which was filed on Jun. 17, 2005, and each of which is expressly incorporated by reference in its entirety.

Briefly, the plasmids are administered intranodally to the inguinal lymph nodes, one to the left side and one to the right, as entraining immunogens. Subsequently the peptides are sequentially administered intranodally, two on separate days to the left node, the other two on separate days to the right as amplifying immunogens. Preferably, the peptides are administered to the same lymph node that received the plasmid encoding the corresponding epitopes. The entrain-and-amplify protocol is described in greater detail in U.S. patent application Ser. No. 10/871,707 (Pub. No. 2005-0079152 A1), filed on Jun. 17, 2004 and 60/479,393, filed on Jun. 17, 2003, each of which is expressly incorporated by reference in its entirety.

Carcinoma patients, especially those with ovarian, colorectal, pancreatic, or renal cell carcinoma, can be screened according to the methods disclosed herein and MKC1106 administered to patients whose tumor profile includes PRAME, PSMA, NY-ESO-1, and/or SSX-2. The NY-ESO-1 epitope targeted by MKC1106 is also found in LAGE 1a/s, so the presence of this antigen in a profile would also be considered a match. As tumor antigen expression tends to be heterogeneous, any particular tissue sample is likely not to give a complete indication of all the antigens expressed. Thus, it is not necessary that a patient's profile contain all four of the antigens for that patient to be a candidate for treatment with MKC1106. However, preferably the profile contains 2, 3, or 4 of the antigens.

What is claimed is:

1. A method of matching a cancer condition in a patient with an immunotherapeutic product, comprising the steps of:
assaying tumor tissue of the patient for two or more expressed tumor-associated antigens (TuAAs) in a preselected panel, wherein the panel includes a growth factor or a signal transduction protein, to develop an antigen profile for the tumor; and
selecting an immunotherapeutic product for the patient based on the profile, wherein the immunotherapeutic agent targets one or more of the expressed antigens in the profile.

2. The method of claim 1, wherein at least one of the TuAAs is selected from the group consisting of a cancer testis antigen, a tissue-specific antigen, an oncofetal antigen, a differentiation antigen, a growth factor, a growth factor receptor, an adhesion factor, a signal transduction protein, a transcription factor, an oncogene product, a tumor suppressor gene product, and a microbial antigen.

3. The method of claim 1, wherein the preselected panel comprises two or more antigens selected from the group consisting of an SSX protein, SSX-2, SSX-4, a MAGE protein, MAGE-1, MAGE-3, PRAME, NY-ESO-1, LAGE, PSMA, PSCA, SCP-1, melan-A/MART-1 and tyrosinase; and wherein the cancer condition is carcinoma.

4. The method of claim 3, wherein the carcinoma is selected from the group consisting of breast, colorectal, prostate, pancreatic, lung, ovarian, renal cell, esophageal, head and neck, and melanocyte.

5. The method of claim 1, wherein the immunotherapeutic product is an active immunotherapuetic.

6. The method of claim 1, wherein the immunotherapeutic product comprises or encodes at least a segment of at least one of the expressed TuAAs.

7. The method of claim 1, wherein the immunotherapeutic product is a passive immunotherapeutic.

8. The method of claim 7, wherein the immunotherapeutic product is a monoclonal antibody.

9. The method of claim 1, comprising at least two assaying steps carried out at different time points during the course of disease, wherein comparative information is obtained from the assaying steps.

10. The method of claim 9, where the obtained information is used to implement, modify or withdraw a therapy.

11. The method of claim 1, wherein the tumor is melanoma and the panel of TuAAs comprises at least two TuAAs selected from the group consisting of tyrosinase, melan-A/MART-1, NY-ESO-1, PRAME, an SSX protein, and a MAGE protein.

12. The method of claim 11, wherein the SSX protein is SSX-2 or SSX-4.

13. The method of claim 11, wherein the MAGE protein is MAGE-1 or MAGE-3.

14. The method of claim 1, wherein the tumor is breast cancer and the panel of TuAAs comprises at least two TuAAs selected from the group consisting of NY-ESO-1, Her2/neu, an SSX protein, and a MAGE protein.

15. The method of claim 1, wherein the tumor is colorectal cancer and the panel of TuAAs comprises at least two TuAAs selected from the group consisting of CEA, an SSX protein, PRAME, NY-ESO, LAGE, PSCA, SCP-1, PSMA and a MAGE protein.

16. The method of claim 1, wherein the tumor is lung cancer and the panel of TuAAs comprises at least two TuAAs selected from the group consisting of PSMA, NY-ESO-1, SSX-2, and a MAGE protein.

17. The method of claim 1, wherein the tumor is prostate cancer and the panel of TuAAs comprises at least two TuAAs selected from the group consisting of NY-ESO-1, PSA, PSCA, PSMA, an SSX protein, and a MAGE protein.

18. The method of claim 1, wherein the tumor is ovarian cancer and the panel of TuAAs comprises at least two TuAAs selected from the group consisting of PRAME, PSMA, PSCA, a MAGE protein, SCP-1, an SSX protein, CEA, Her-2/Neu, NY-ESO-1, and LAGE.

19. The method of claim 18, wherein the ovarian cancer is selected from the group consisting of serous carcinoma, non-serous carcinoma, mucinous (cell) carcinoma, and clear cell carcinoma.

20. The method of claim 1, wherein the tumor is renal cancer and the panel of TuAAs comprises at least two TuAAs selected from the group consisting of an SSX protein, PRAME, NY-ESO, LAGE, PSCA, SCP-1, PSMA and a MAGE protein.

21. The method of claim 1, wherein the tumor is pancreatic cancer and the panel of TuAAs comprises at least two TuAAs selected from the group consisting of an SSX protein, PRAME, NY-ESO, LAGE, PSCA, PSMA and a MAGE protein.

22. The method of claim 1, wherein antigen expression is detected by a technique comprising at least one of RT-PCR, transcript determination, protein determination, epitope determination or any combination thereof.

23. The method of claim 1, wherein antigen expression is detected on neoplastic cells, or tumor-associated stromal cells, or both.

24. The method of claim 23, wherein the tumor-associated stromal cells are neovasculature.

25. The method of claim 24, wherein the neovasculature-associated antigen is PSMA and the neoplastic cell antigen is selected form the group consisting of NY-ESO-1, SSX2, LAGE, and PRAME.

26. The method of claim 1, wherein the tumor tissue comprises primary tumor tissue.

27. The method of claim 1, wherein the tumor tissue comprises metastatic tumor tissue.

28. The method of claim 1, wherein said growth factor is VEGF-A.

29. The method of claim 1, wherein said signal transduction protein is PLK1.

30. A method of matching a cancer condition in a patient with an immunotherapeutic regimen, comprising the steps of:
assaying the patient's tumor tissue for two or more expressed tumor-associated antigens (TuAAs) in a preselected panel wherein the panel includes a growth factor or a signal transduction protein, to develop an antigen profile for the tumor; and
selecting an immunotherapeutic regimen based on the profile, the regimen comprising administration of one or more immunotherapeutic agents targeting two or more antigens in the profile.

31. The method of claim 30, wherein the regimen comprises administering both an active immunotherapeutic agent and a passive immunotherapeutic agent.

32. The method of claim 30, wherein said growth factor is VEGF-A.

33. The method of claim 30, wherein said signal transduction protein is PLK1.

34. The method of claim 30, wherein the diagnosis is colon cancer and the lineage marker is CEA.

35. The method of claim 30, wherein the diagnosis is lung cancer and the lineage marker is thyroid transcription factor 1 (TTF1).

36. The method of claim 30, wherein the diagnosis is prostate cancer and the lineage marker is selected from the group consisting of PSA and PSMA.
